(19) United States
(12) Patent Application Publication
(10) Pub. No.: US 2004/0063731 A1
(43) Pub. Date: Apr. 1, 2004

(54) Pharmaceutical formulation comprising pyrazolo[4,3-d]pyrimidines and endothelin receptor antagonists or thienopyrimidines and endothelin receptor antagonists

(76) Inventors: Hans-Michael Eggenweiler, Darmstadt (DE); Volker Eiermann, Darmstadt (DE); Pierre Schelling, Darmstadt (DE)

Correspondence Address: Millen White Zelano & Branigan, Arlington Courthouse Plaza I Suite 1400, 2200 Clarendon Blvd, Arlington, VA, 22201, US

(21) Appl. No.: 10/470,763

(22) PCT Filed: Jan. 14, 2002
(86) PCT No.: PCT/EP02/00256

(30) Foreign Application Priority Data
Feb. 2, 2001 (DE) 10104800.9
Feb. 2, 2001 (DE) 10104801.7
Feb. 2, 2001 (DE) 10104802.5

(51) Int. Cl. A61K 31/519

(52) U.S. Cl. 514/262.1

(57) ABSTRACT

Pharmaceutical preparation comprising at least one phosphodiesterase V inhibitor have, and/or physiologically acceptable salts and/or solvates thereof and at least one endothelin receptor antagonist for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

The invention relates to pharmaceutical formulations comprising at least one phosphodiesterase V inhibitor and/or physiologically acceptable salts and/or solvates thereof and at least one endothelin receptor antagonist.

The invention relates in particular to pharmaceutical formulations comprising at least one compound of the formula I

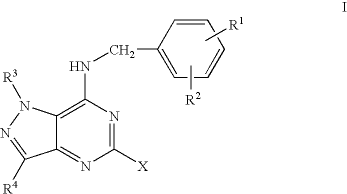

in which

R¹ and R² are each, independently of one another, H, A, OH, OA or Hal,

R¹ and R² together are alternatively alkylene having 3-5 carbon atoms, —O—CH₂—CH₂—, —CH₂—O—CH₂—, —O—CH₂—O— or —O—CH₂—CH₂—O—,

R⁵ and R⁴ are each, independently of one another, H or A,

X is R⁵, R⁶ or R⁷, each of which is monosubstituted by R⁸,

R⁵ is linear or branched alkylene having 1-10 carbon atoms, in which one or two CH₂ groups may be replaced by —CH═CH— groups, O, S or SO,

R⁶ is cycloalkyl or cycloalkylalkylene having 5-12 carbon atoms,

R⁷ is phenyl or phenylmethyl,

R^{a} is COOH, COOA, CONH₂, CONHA, CON(A)₂ or CN,

A is alkyl having from 1 to 6 carbon atoms, and

Hal is F, Cl, Br or I,

and/or physiologically acceptable salts and/or solvates thereof and at least one endothelin receptor antagonist.

The invention furthermore relates to pharmaceutical formulations comprising at (east one phosphodiesterase V inhibitor and/or physiologically acceptable salts and/or solvates thereof and at least one endothelin receptor antagonist.

The invention relates in particular to pharmaceutical formulations comprising at least one compound of the formula I-I

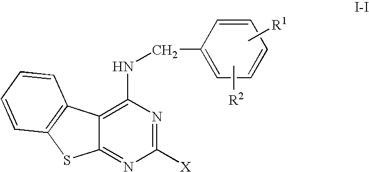

in which

R¹ and R² are each, independently of one another, H, A, OA, OH or Hal,

R¹ and R² together are alternatively alkylene having 3-5 carbon atoms, —O—CH₂—CH₂—, —CH₂—O—CH₂—, —O—CH₂—O— or —O—CH₂—CH₂—O—,

X is R⁴, R⁵ or R⁶, each of which is monosubstituted by R⁷,

R⁴ is linear or branched alkylene having 1-10 carbon atoms, in which one or two CH₂ groups may be replaced by —CH═CH— groups,

R⁵ is cycloalkyl or cycloalkylalkylene having 5-12 carbon atoms,

R⁶ is phenyl or phenylmethyl,

R⁷ is COOH, COOA, CONH₂, CONHA, CON(A)₂ or CN, A is alkyl having from 1 to 6 carbon atoms, and

Hal is F, Cl, Br or I,

and/or physiologically acceptable salts and/or solvates thereof and at least one endothelin receptor antagonist.

The invention furthermore relates to pharmaceutical formulations comprising at least one phosphodiesterase V inhibitor and/or physiologically acceptable salts and/or solvates thereof and at least one endothelin receptor antagonist.

The invention relates in particular to pharmaceutical formulations comprising at least one compound of the formula I-II

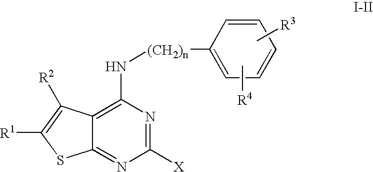

in which

R¹ and R² are each, independently of one another, H, A or Hal, where one of the radicals R¹ or R² is always≠H,

R¹ and R² together are alternatively alkylene having 3-5 carbon atoms,

R³ and R⁴ are each, independently of one another, H, A, OH, OA or Hal,

R³ and R⁴ together are alternatively alkylene having 3-5 carbon atoms, —O—CH₂—CH₂—, —O—CH₂—O— or —O—CH₂—CH₂—O—,

X is R⁵ or R⁶, each of which is monosubstituted by R⁷,

R⁵ is linear or branched alkylene having 1-10 carbon atoms, in which one or two CH₂ groups may be replaced by —CH—CH— groups, or C₆H₄(CH₂)ₘ—,

R⁶ is cycloalkylalkylene having 6-12 carbon atoms,

R⁷ is COOH, COOA, CONH₂, CONHA, CON(A)₂ or CN,

A is alkyl having from 1 to 6 carbon atoms,

Hal is F, Cl, Br or 1,

m is 1 or 2, and

n is 0, 1, 2 or 3,

and/or physiologically acceptable salts and/or solvates thereof and at least one endothelin receptor antagonist.

The invention furthermore relates to the use of the formulation for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

Pharmaceutical formulations consisting of other phosphodiesterase V (PDE V) inhibitors together with a second active ingredient are described in WO 00/15639. Combinations of PDE V inhibitors with endothelin receptor antagonists are also described, for example, in WO 99/64004.

The use of other PDE V inhibitors is described, for example, in WO 94/28902.

The invention was based on the object of providing novel medicaments in the form of pharmaceutical preparations which have better properties than known medicaments which can be used for the same purpose.

This object has been achieved by the discovery of the novel preparation.

The compounds of the formulae I,I-I and I-II and their salts have very valuable pharmacological properties and are well tolerated. In particular, they exhibit specific inhibition of cGMP phosphodiesterase (PDE V).

Quinazolines having a cGMP phosphodiesterase-inhibiting activity are described, for example, in J. Med. Chem. 36, 3765 (1993) and ibid. 37, 2106 (1994).

The biological activity of the compounds of the formulae I, I-I and I-II can be determined by methods as described, for example, in WO 93/06104. The affinity of the compounds according to the invention for cGMP and cAMP phosphodiesterase is determined by measuring their IC₅₀ values (concentration of the inhibitor needed to achieve 50% inhibition of the enzyme activity). The determinations can be carried out using enzymes isolated by known methods (for example W.J. Thompson et al., Biochem. 1971, 10, 311). The experiments can be carried out using a modified batch method of W.J. Thompson and M.M. Appleman (Biochem. 1979, 18, 5228). The compounds are therefore suitable for the treatment of illnesses of the cardiovascular system, in particular cardiac insufficiency, and for the treatment and/or therapy of potency disorders (erectile dysfunction).

The use of substituted pyrazolopyrimidinones for the treatment of impotence is described, for example, in WO 94/28902.

The compounds are effective as inhibitors of phenylephrine-induced contractions in corpus cavernosum preparations of rabbits. This biological action can be demonstrated, for example, by the method described by F. Holmquist et al. in J. Urol., 150, 1310-1315 (1993).

The inhibition of the contraction demonstrates the effectiveness of the compounds according to the invention for the therapy and/or treatment of potency disorders.

The efficacy of the pharmaceutical formulations according to the invention, in particular for the treatment of high pulmonary pressure, can be demonstrated as described by E. Braunwald in Heart Disease 5t edition, W B Saunders Company, 1997, chapter 6: Cardiac catheterisation 177-200.

The compounds of the formulae I, I-I and I-II can be employed as medicament active ingredients in human and veterinary medicine. They can furthermore be employed as intermediates for the preparation of further medicament active ingredients.

Compounds of the Formula I

The compounds of the formula I according to claim 1 and their salts are prepared by a process

characterised in that

a) a compound of the formula 11

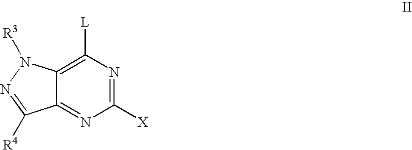

in which

R³, R⁴ and X are as defined above,

and L is Cl, Br, OH, SCH₃ or a reactive esterified OH group,

is reacted with a compound of the formula III

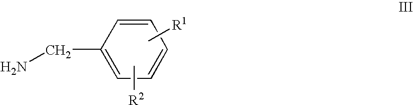

in which

R¹ and R² are as defined above,

or

b) a radical X in a compound of the formula I is converted into another radical X by, for example, hydrolysing an ester group to a COOH group or converting a COOH group into an amide or into a cyano group, and/or in that a compound of the formula I is converted into one of its salts.

The term solvates of the compounds of the formula I is taken to mean adductions of inert solvent molecules onto the compounds of the formula I which form owing to their mutual attractive force. Solvates are, for example, mono- or dihydrates or alcoholates.

Above and below, the radicals R¹, R², R³, R⁴, R⁶, R^{6′, R}⁷, R⁸, X and L have the meanings indicated for the formulae I, II and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁵, R⁶ or R⁷ radical which is monosubstituted by R⁸.

R⁶ is a linear or branched alkylene radical having 1-10 carbon atoms, where the alkylene radical is preferably, for example, methylene, ethylene, propylene, isopropylene, butylene, isobutylene, sec-butylene, pentylene, 1-2- or 3-methylbutylene, 1,1-, 1,2- or 2,2-dimethylpropylene, 1-ethylpropylene, hexylene, 1-, 2-, 3- or 4-methylpentylene, 1,1-, 1,2-, 1,3-, 2,2-, 2,3- or 3,3dimethylbutylene, 1- or 2-ethylbutylene, 1-ethyl-1-methylpropylene, 1-ethyl-2-methylpropylene, 1,1,2- or 1,2,2-trimethylpropylene, linear or branched heptylene, octylene, nonylene or decylene.

R⁵ is furthermore, for example, but-2-enylene or hex-3-enylene.

One CH₂ group in R⁵ may preferably be replaced by oxygen.

Very particular preference is given to ethylene, propylene, butylene or CH₂—O—CH₂.

R⁶ is cycloalkylalkylene having 5-12 carbon atoms, preferably, for example, cyclopentylmethylene, cyclohexylmethylene, cyclohexylethylene, cyclohexylpropylene or cyclohexylbutylene.

R⁶ is alternatively cycloalkyl, preferably having 5-7 carbon atoms. Cycloalkyl is, for example, cyclopentyl, cyclohexyl or cycloheptyl.

Hal is preferably F, Cl or Br, but alternatively I.

The radicals R¹ and R² may be identical or different and are preferably in the 3- or 4-position of the phenyl ring. They are, for example, each, independently of one another, H, alkyl, OH, F, Cl, Br or I or together are alkylene, such as, for example, propylene, butylene or pentylene, furthermore ethyleneoxy, methylenedioxy or ethylenedioxy. They are alternatively preferably each alkoxy, such as, for example, methoxy, ethoxy or propoxy.

The radical R⁶ is preferably, for example, COOH, COOA, such as, for example, COOCH, or COOC₂H₅, CONH₂, CON(CH₃)₂, CONHCH₃ or CN, but in particular COOH or COOA.

Throughout the invention, all radicals which occur more than once may be identical or different, i.e. are independent of one another.

The invention relates, in particular, to pharmaceutical formulations comprising an endothelin receptor antagonist and at least one compound of the formula I in which at least one of the said radicals has one of the preferred meanings indicated above. Some preferred groups of compounds may be expressed by the following sub-formulae Ia to If, which conform to the formula I and in which the radicals not designated in greater detail have the meaning indicated for the formula 1, but in which

in Ia X is R⁵, phenyl or phenylmethyl, each of which is substituted by COOH, COOA, CONH₂, CONA₂, CONHA or CN;

in Ib R¹ and R² together are alkylene having 3-5 carbon atoms, —O—CH₂—CH₂′—, —O—CH₂—O— or —O—CH₂—CH₂—O,

X is R⁵, phenyl or phenylmethyl, each of which is substituted by COOH, COOA, CONH₂, CONA₂, CONHA or CN;

in Ic

R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

X is R , phenyl or phenylmethyl, each of which is substituted by COOH, COOA, CONH₂, CONA₂, CONHA or CN;

in Id

R¹ and R² are each, independently of one another, H. A, OH, OA or Hal,

R¹ and R² together are alternatively alkylene having 3-5 carbon atoms, —O—CH₂—CH₂—, —O—CH₂—O— or —O—CH₂—CH₂—O—,

X is alkylene having 2-5 carbon atoms, cyclohexyl, phenyl or phenylmethyl, each of which is monosubstituted by R⁸,

R³ is alkyl having 1-6 carbon atoms,

R⁴ is alkyl having 1-6 carbon atoms,

R⁸ is COOH or COOA,

A is alkyl having from 1 to 6 carbon atoms,

Hal is F, Cl, Br or I;

in Ie

R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

R³ is alkyl having 1-6 carbon atoms,

R⁴ is alkyl having 1-6 carbon atoms,

X is —(CH₂)₂₋₅—R⁸, 4-R⁸-cyclohexyl, 4-R³-phenyl or 4-(R⁸-methyl)phenyl;

in If

R¹ and R² are each, independently of one another, H, A, OH, OA or Hal,

R¹ and R² together are alternatively alkylene having 3-5 carbon atoms, —O—CH₂—CH₂—, —O—CH₂—O— or —O—CH₂—CH₂—O—,

R³ is alkyl having 1-6 carbon atoms,

R⁴ is alkyl having 1-6 carbon atoms,

X is —(CH₂)₂₋₅—R⁸, in which one CH₂ group may be replaced by 0, or is 4-R⁸-cyclohexyl, 4-R⁶-phenyl or 4-(R⁸-methyl)phenyl,

R⁸ is COOH or COOA.

The invention preferably relates to a formulation comprising [7-(3-chloro-4-methoxybenzylamino)-1-methyl-3-propyl-1H-pyrazolo[4,3-d]pyrimidin-5-yl-methoxy]acetic acid and physiologically acceptable salts and/or solvates thereof and an endothelin receptor antagonist.

Besides the free acid, the ethanolamine salt is preferred.

Preferred endothelin receptor antagonists are bosentan, tezosentan and sitaxentan (TBC-11251; J.Med.Chem., 40, No.11,1690-97, 1997). Preferred endothelin receptor antagonists are thus furthermore

a) BMS-193884 (EP 558258),

b) BMS-207940 (Pharmaprojects (13.06.97)),

c) BQ-123 (Exp.Opin.Invest.Drugs, 1997, 6, No.5, 475-487),

d) SB-209670 (Exp.Opin.Invest.Drugs, 1997, 6, No.5, 475-487),

e) SB-217242 (Exp.Opin.Invest.Drugs, 1997, 6, No.5, 475487),

f) SB-209598 (Trends in Pharmacol. Sci., 17,177-81,1996),

g) TAK-044 (Exp.Opin.Invest.Drugs, 1997, 6, No.5, 475-487),

h) Bosentan (Trends in Pharmacol. Sci., 18, 408-12, 1997),

i) PD-156707 (J.Med.Chem., 40, No.7,1063-74, 1997),

j) L-749329 (Bioorg.Med.Chem.Lett., 7, No.3, 275-280,1997),

k) L-754142 (Exp.Opin.Invest.Drugs, 1997, 6, No.5, 475-487),

l) ABT-627 (J.Med.Chem., 40, No.20, 3217-27,1997),

m) A-127772 (J.Med.Chem., 39, No.5, 1039-1048, 1996),

n) A-206377 (213^{th} American Chemical Society National Meeting, San Francisco, Calif., USA, 13-17 April 1997, Poster, MEDI 193),

o) A-182086 (J.Med.Chem., 40, No.20, 3217-27,1997),

p) EMD-93246 (211^{th} American Chemical Society National Meeting, New Orleans, USA, 1996, Poster, MEDI 143),

q) EMD-122801. (Bioorg.Med.Chem.Lett., 8, No.1, 17-22,1998),

r) ZD-1611 (Trends in Pharmacol. Sci., 18, 408-12,1997),

s) AC-610612 (R&D Focus Drug News (18.05.98)),

t) T-0201 (70t Annual Meeting of the Japanese Pharmacological Society, Chiba, Japan, 22-15 March 1997, Lecture, O-133),

u) J-104132 (R&D Focus Drug News (15.12.97)),

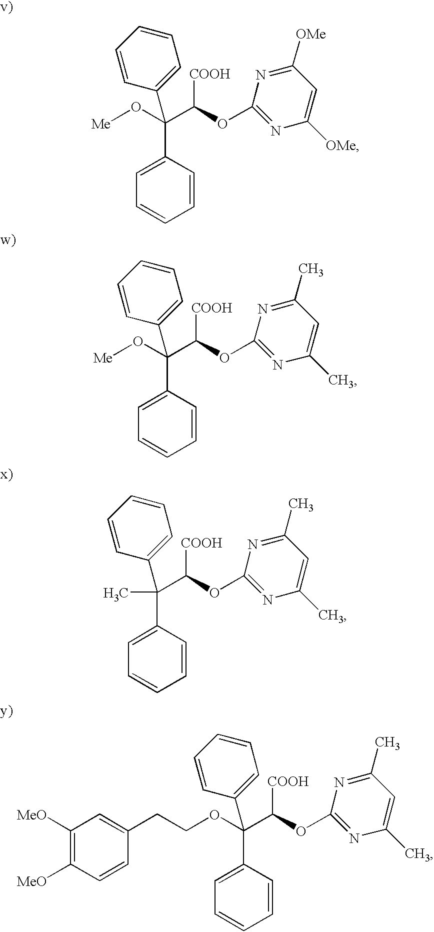

Particularly preferred endothelin receptor antagonists are, for example,

a) the compounds of the formula I described in EP 0733626

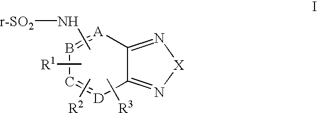

in which

-A=B-C=D- is a —CH═CH—CH═CH— group in which 1 or 2 CH has (have) been replaced by N,

Ar is Ph or naphthyl, each of which is unsubstituted or monosubstituted, disubstituted or trisubstituted by H, Hal, A, alkenyl having up to 6 carbon atoms, Ph, OPh, NO₂, NR⁴R⁵, NHCOR⁴, CF₃, OCF₃, CN, OR⁴, COOR⁴, (CH₂),COOR⁴, (CH₂)ₙNR¹R⁵, —N═C═O or NHCONR⁴R⁵,

R¹, R²

and R³ are each, independently of one another, absent, H, Hal, A, CF₃, NO₂, NR⁴R⁵, CN, COOR⁴, NHCOR⁴,

R⁴ and R⁵ are each, independently of one another, H or A, or together are alternatively —CH₂—(CH₂), —CH₂—,

A is alkyl having from 1 to 6 carbon atoms,

Ph is phenyl,

X is O or S,

Hal is F, Cl, Br or 1,

n is 1, 2 or 3,

and their salts, with the exception of

4-methyl-N-(2,1,3-benzothiadiazol-4-yl)benzenesulfonamide,

4-methyl-N-(2,1,3-benzothiadiazol-5-yl)benzenesulfonamide, 4-nitro-N-(2,1,3-benzothiadiazol-4-yl)benzenesulfonamide, 4-nitro-N-(2,1,3-benzothiadiazol-5-yl)benzenesulfonamide, 4-amino-N-(2,1,3-benzo-thiadiazol-4-yl)benzenesulfonamide and 4-amino-N-(2,1,3-benzothiadiazol-5-yl)benzenesulfonamide;

b) The Compounds of the Formula I Described in EP 0733626

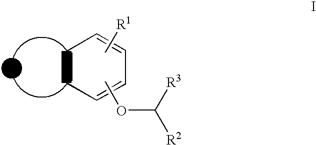

in which

X is a saturated, partially unsaturated or completely unsaturated 3- to 4-membered alkylene chain, in which from 1 to 3 carbon atoms may be replaced by N and/or from 1 to 2 carbon atoms may be replaced by 1-20 atoms and/or 1-2 S atoms, but where at most up to 3 carbon atoms may be replaced and where, in addition, a single, double or triple substitution of the alkylene chain and/or of a nitrogen located therein by A, RB and/or NR⁴R^{4′} may occur, and where furthermore one CH₂ group in the alkylene chain may also be replaced by a C═O group,

A is alkyl having 1-6 carbon atoms, in which one or two CH₂ groups may be replaced by O or S atoms or by —CR⁴═CR⁴— groups and in addition 1-7H atoms may be replaced by F,

R¹ is H or A,

R² is COOR⁴, CN, 1H-tetrazol-5-yl or CONHSO₂R⁸,

R³ is Ar,

R⁴ and R^{4′} are each, independently of one another, H, alkyl having from 1 to 6 carbon atoms or benzyl,

Ar is phenyl or naphthyl, each of which is unsubstituted or monosubstituted, disubstituted or trisubstituted by R⁵, Re or R⁷,or is a

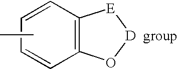

which is unsubstituted or monosubstituted or disubstituted in the phenyl part by R⁵ or R⁸,

R⁵, R⁵ and R⁷ are each, independently of one another, R⁴, OR⁴, Hal, CF₃, OCF₃, OCHF₂, OCH₂F, NO₂, NR⁴R⁴′, NHCOR⁴, CN,NHSO₂R⁴, COOR⁴, COR⁴, CONHSO₂R⁸, O(CH₂)ₙR², OPh, O(CH₂)ₙOR⁴ or S(O)ₘR⁴,

R⁸ is phenyl or naphthyl, each of which is unsubstituted or monosubstituted, disubstituted or trisubstituted by A, OR¹, NR⁴R^{4′} or Hal,

E is CH₂ or O,

D is carbonyl or [C(R⁴R⁴)],

Hal is F, Cl, Br or I,

m is 0, 1 or 2,

n is 1 or 2,

and their salts;

c) The Compounds of the Formula I Described in EP 0755934

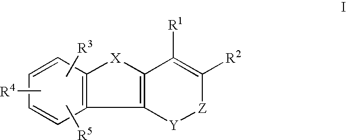

in which

—Y-Z— is —NR—CO—, —N═C(OR)— or —N═CR⁸—,

R¹ is Ar,

R² is COOR⁶, CN, 1H-tetrazol-5-yl or CONHSO₂Ar,

R³, R⁴ and R⁵ are each, independently of one another, R⁶, OR⁸, S(O),R⁶, Hal, NO₂, NR⁸R⁶′, NHCORW, NHSO₂R⁸, OCOR⁶, COOR⁶ or CN,

R⁶ and R^{6′} are each, independently of one another, H, alkyl having from 1 to 6 carbon atoms, benzyl or phenyl,

R⁷ is (CH₂)ₙAr,

R⁸ is Ar or OAr,

Ar is phenyl which is unsubstituted or monosubstituted, disubstituted or trisubstituted by R⁹, R¹⁰ or R¹¹, or is unsubstituted naphthyl or a

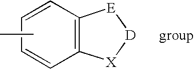

which is unsubstituted or monosubstituted or disubstituted in the phenyl part by R⁹ or R¹⁰, or is a

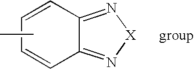

which is unsubstituted or monosubstituted or disubstituted in the cyclohexadienyl part by R⁹ or R¹⁰,

R⁹, R¹⁰ and R¹¹ are each, independently of one another, R⁶, OR⁶, Hal, CF₃, OCF₃, OCHF₂, OCH₂F, NO₂, NR⁶R⁶′, NHCOR⁶, CN, NHSO₂R⁸, COOR⁶, COR⁶, CONHSO₂Ar, O(CH₂)ₙR², O(CH₂)ₙOR⁶ or S(O)ₘR⁶,

E is CH₂, S or O,

D is carbonyl or [C(R⁶R^{6′})]ₙ,

Hal is F, Cl, Br or I,

X is O or S,

m is 0, 1 or 2,

n is 1 or 2,

and their salts;

d) The Compounds of the Formula I Described in EP 0757039

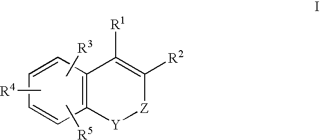

in which

—Y-Z— is —NR⁷—CO—, —N═C(OR⁷)— or —N═CR⁶—,

R¹ is Ar,

R² is COOR⁶, (CH₂)ₙCOOR⁶, CN, 1H-tetrazol-5-yl or CONHSO₂Ar,

R³, R⁴ and R⁵ are each, independently of one another, R⁶, OR⁶, S(O)ₘR⁶, Hal, NO₂, NR⁵R⁶R⁶, NHCOR⁶, NHSO₂R⁶, OCOR⁶, COR⁶, COOR⁶ or CN, where R³ and R⁴ together may alternatively be an O(CH₂)O group,

R⁶ and R^{6′} are each, independently of one another, H, alkyl having from 1 to 6 carbon atoms, benzyl or phenyl,

R⁷ is (CH₂),Ar,

R⁸ is Ar or OAr,

Ar is phenyl which is unsubstituted or monosubstituted, disubstituted or trisubstituted by R⁹, R¹⁰ or R¹¹, or is unsubstituted naphthyl or a

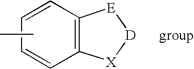

which is unsubstituted or monosubstituted or disubstituted in the phenyl part by R⁹ or R¹⁰, or is a

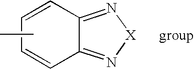

which is unsubstituted or monosubstituted or disubstituted in the cyclohexadienyl part by R⁹ or R¹⁰, R⁹, R¹⁰ and R¹¹ are each, independently of one another, R⁶W, OR, Hal, CF₃, OCF₃, OCHF₂, OCH₂F, NO₂, NR⁶R^{6′}, NHCOR⁶, CN, NHSO₂R⁸, COOR⁶, COR^{6′}, CONHSO₂Ar, O(CH₂)ₙR², O(CH₂),OR⁶ or S(O)ₘR¹,

E is CH₂, S or O,

D is carbonyl or [C(R¹R⁵)]ₙ,

X is O or S,

Hal is F, Cl, Br or 1,

m is 0, 1 or 2,

n is 1 or 2,

and their salts;

e) The Compounds of the Formula I Described in EP 0796250

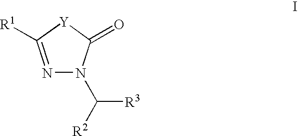

in which

Y is —C(R⁴R⁴)—C(R⁴R^{4′})—, —CR⁴═CR⁴— or —C(R⁴R^{4′})—S—,

R¹ is Het, Ar, R³ or R⁴,

R² is Ar or a

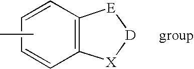

which is unsubstituted or monosubstituted or disubstituted in the phenyl part by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘR⁴,

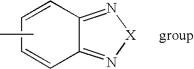

which is unsubstituted or monosubstituted or disubstituted in the cyclohexadienyl part by A, R³, OR⁴, NH₂, NHA, NA₂, NO₂, CN, Hal, NHCOR⁴, NHSO₂R⁴, COOR⁴, COR⁴, CONHSO₂R^{6′} O(CH₂)ₙR³, OPh, O(CH₂)ₙOR⁴ or S(O)ₘR⁴,

R³ is CN, COOH, COOA, CONHSO₂R⁶ or 1H-tetrazol-5-yl,

R⁴ and R^{4′} are each, independently of one another, H, A, or phenyl or benzyl, each of which is unsubstituted or monosubstituted by alkoxy,

R⁵ is A or Ar,

R⁶ is phenyl or naphthyl, each of which is unsubstituted or monosubstituted, disubstituted or trisubstituted by A, OR⁵, NH₂, NHA, NA₂, NO₂, CN or Hal,

A is alkyl having 1-6 carbon atoms, in which one or two CH₂ groups may be replaced by O or S atoms or by —CR⁴═CR⁴′-groups and in addition 1-7H atoms may be replaced by F, or benzyl,

Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂R⁴, COOR⁴, COR⁴, CONHSO₂R⁶, O(CH₂)ₙRa, OPh, O(CH₂)ₙOR⁴ or S(O)ₘR⁴,

Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂

- and/or carbonyl oxygen,

D is carbonyl or [C(R⁴R⁴)],

E is CH₂, S or O,

Hal is F, Cl, Br or 1,

X is O or S,

m is 0, 1 or 2,

n is 1 or 2,

and their salts;

f) the compounds of the formula I described in WO 9719077

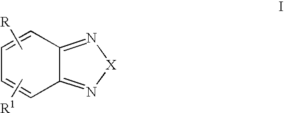

in which

R

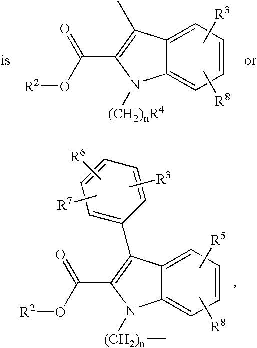

X is O or S,

R¹ is H, Hal, OH, OA, A, alkylene-O-A, NO₂, NH₂, NH-acyl, SO₂NH₂, SO₃-A, SO₂NHA, CN or formyl,

R² is H or A,

R³, R⁵, R⁵

R⁷ and R¹ are each, independently of one another, H, Hal, OH, OA, O-alkylene-R⁴, A, S-A, NO₂, NH₂, NHA, NA₂, NH-acyl, NHSO₂A, NHSO₂R⁴, NASO₂A, NASO₂—R⁴, NH(CO)NH₂, NH(CO)NHA, formyl, NH(CO)NH-phenyl,

NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CH₂)ₙOWR, CH₂OH or CH₂OA,

R³ and R⁴ together are alternatively —O—CH₂—O—O—CH₂—CH₂—O—, —O—CH₂—CH₂—, —O—CF₂—O— or —O—CF₂—CF₂—O—,

R⁴ is phenyl which is unsubstituted or monosubstituted or polysubstituted by R³ and/or R⁸,

A is alkyl having 1-6 carbon atoms,

Hal is fluorine, chlorine, bromine or iodine,

n is 1 or 2,

and their salts;

g) the compounds of the formula I described in WO 9730982

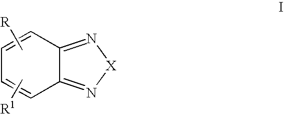

in which

R is

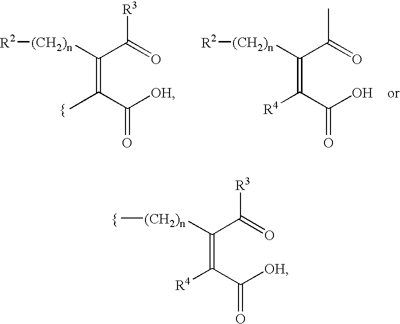

x is O or S,

R¹ is H, Hal, OH, OA, A, alkylene-O-A, NO₂, NH₂, NH-acyl, SO₂NH₂, SO₃-A, SO₂NHA, CN or formyl,

R², R³

and R⁴ are each, independently of one another, a phenyl group which is unsubstituted or monosubstituted or polysubstituted by Hal, OH, OA, O-alkylene-R⁵, A, S-A, SOA, SO₂A, S)R⁵, SO₂R⁵, NO₂, NH₂, NHA, NA₂, NH:acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂,1-piperidinyl-CO—NH, 1-pyrrolidinyl-CONH, O(CH₂),COOA, O(CH₂),COOH, O(CH₂),OH, O(CH₂)ₙOA, CH₂OH, CH₂OA, COOH, COOA, CH₂COOH or CH₂COOA,

or is a

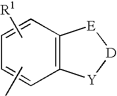

group or a

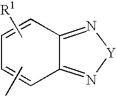

group, where

R² is additionally A or cycloalkyl,

R⁶ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙ,COOA, O(CH₂)ᵣCOOH, O(CH₂),OH, O(CH₂),OA, CH₂OH, CH₂OA, COOH, COOA, CH₂COOH or CH₂COOA,

A is alkyl having 1-6 carbon atoms, in which one or two CH₂ groups may be replaced by O or S atoms or by —CR⁶═CR′″-groups and/or 1-7H atoms may be replaced by F,

D is carbonyl or [C(R⁶R^{6′})]ₘ,

E is CH₂, S or O,

Y is O or S,

R⁶ and R^{6′} are each, independently of one another, H, F or A,

Hal is fluorine, chlorine, bromine or iodine,

n is 1 or 2, and

m is 1 or 2,

or a tautomeric cyclised form, and the (E)-isomers and the salts of all isomers;

h) the compounds of the formula I described in WO 9730996

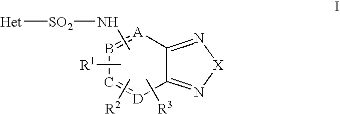

in which

-A=B-C=D- is a —CH═CH—CH═CH— group, in which, in addition, 1 or 2 CH may be replaced by N,

Het is a monocyclic or bicyclic, saturated, unsaturated or aromatic heterocyclic radical having from 1 to 4 N, O and/or S atoms which is unsubstituted or substituted by —Z—R⁶,

R¹,R²

and R³ are each, independently of one another, absent, H, Hal, A, CF₃, NO₂, NR⁴R⁵, CN, COOR⁴ or NHCOR⁴,

R⁴ and R⁵ are each, independently of one another, H or A, or together are alternatively —CH₂—(CH₂), —CH₂—,

R⁶ is a phenyl radical, benzothiadiazol-5-yl or benzoxadiazol-5-yl radical, each of which is unsubstituted or monosubstituted, disubstituted or trisubstituted by R⁷, R¹ and/or R⁹,

R⁷, R⁸

and R⁹ are each, independently of one another, A, O-A, CN, COOH, COOA, Ha), formyl, —CO-A, and R⁷ and R^{7′} are alternatively —O—(CH₂)ₘ—O—,

A is alkyl having from 1 to 6 carbon atoms,

X is O or S,

Z is —CO—, —CONH—, —CO—(CH₂)ₙ—, —CH═CH—, —(CH₂)ₙ—, —CONHCO—, —NHCONH—, —NHCOO—, —O—CONH—, —CO—O— or —O—CO—,

Hal is F, Cl, Br or 1,

m is 1 or 2, and

n is 1, 2 or 3,

and their salts;

i) the compounds of the formula I described in DE 19609597

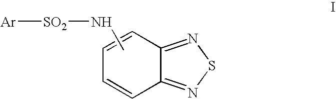

in which

Ar is naphthyl which is monosubstituted by NH₂, NHA or NA₂, and

A is alkyl having from 1 to 6 carbon atoms,

and their physiologically acceptable salts;

j) the compounds of the formula I described in DE 19612101

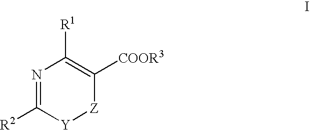

in which

—Y-Z- is —NR⁴—CO or —N═CR⁵—,

R¹ is Ar,

R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by R³, OR³ or Hal,

R³ and R³ are each, independently of one another, H, alkyl having 1-6 carbon atoms or benzyl,

R⁴ is CH₂Ar,

R⁵ is OCH₂Ar,

Ar is phenyl which is unsubstituted or monosubstituted, disubstituted or trisubstituted by R⁶, R⁷ or R⁸, or a

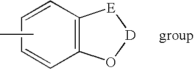

which is unsubstituted or monosubstituted in the phenyl part by R⁶, or a

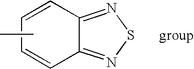

which is unsubstituted or monosubstituted in the cyclohexadienyl part by R⁶,

E is CH₂ or O,

D is carbonyl or (CH₂)ₙ,

E and D together are alternatively CH═CR⁹,

R⁶, R^{6′} are each, independently of one another, R³, OR³ or Hal,

R⁷ is R³, OR³, Hal, NO₂, NH₂, NHR³, NR³R³, NHCOR³, COOR³, O(CH₂),R³ or O(CH₂),OR³,

R⁶ is Ph which is unsubstituted or monosubstituted, disubstituted or trisubstituted by R³, OR³, Hal, NO₂, NH₂, NHR⁶, NR⁶R⁶, NHCOR³ or COOR³,

R⁹ is H, OH, CH₂OH or COOR³,

Hal is F, CI, Br or 1,

Ph is phenyl,

m is 0 or 1,

n is 1 or 2,

and their salts;

k) the compounds of the formula I described in WO 9827091

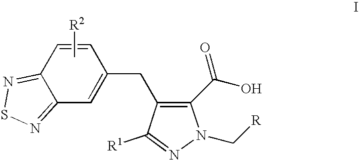

in which

R is phenyl which is unsubstituted or monosubstituted, disubstituted or trisubstituted by R³, R⁴ or R⁵, or 2,1,3-benzothiadiazolyl which is unsubstituted or mono-substituted by R²,

R¹ is A, in which 1-7H atoms may be replaced by F,is —S-A, —O-A, is phenyl or -alkylene-phenyl, each of which is unsubstituted or monosubstituted by R³, or is thienyl which is unsubstituted or monosubstituted by R³,

R² is A, F, Cl, Br or —O-A,

R³, R⁴

and R⁵ are each, independently of one another, A, —O-A, —S-A, —O-alkylene-COOH, -alkylene-COOH or COOH,

R³ and R⁴ together are alternatively —O—CH₂—O—, and

A is alkyl having 1-7 carbon atoms, and their salts;

I) the compounds of the formula I described in WO 9827077

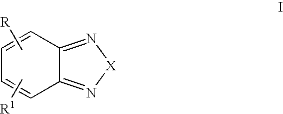

in which

R is

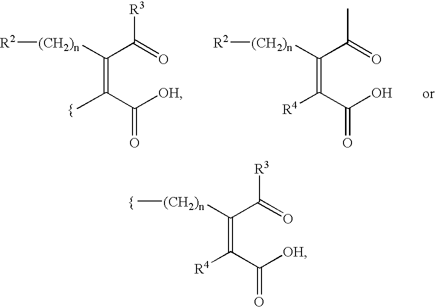

X is O or S,

R¹ is H, Hal, OH, OA, A, alkylene-O-A, NO₂, NH₂, NH-acyl, SO₂NH₂, SO₃-A, SO₂NHA, CN or formyl,

R²,R³

and R⁴ are each, independently of one another, a phenyl group which is unsubstituted or monosubstituted or poly-substituted by R⁷, where R² is additionally A or cycloalkyl, or are

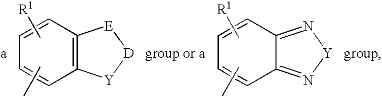

with the proviso that at least one of the radicals R², R³ or R⁴ is an R⁸ radical which is unsubstituted or mono-substituted or polysubstituted by R⁷,

R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂),COOA, O(CH₂),COOH, O(CH₂),OH, O(CH₂),OA, CH₂OH, CH₂OA, COOH, COOA, CHCOOH or CH₂COOA,

A is alkyl having 1-6 carbon atoms, in which one or two CH₂ groups may be replaced by O or S atoms or by —CR⁶═CR^{6′}-groups and/or 1-7H atoms may be replaced by F,

D is carbonyl or [C(R⁶R⁶)]ₘ,

E is CH₂,S or O,

Y is O or S,

R⁶ and R⁶ are each, independently of one another, H. F or A,

R⁷ is Hal, OH, OA, O-alkylene-R¹, A, S-A, S-OA, SO₂A, S—OR⁶, SO₂R⁵, NO₂, NH₂, NHA, NA₂, NH-acyl, NHSO₂A, NHSO₂RW, NASO₂A, NASO₂—R⁵, NH(CO)NH₂, NH(CO)NHA, formyl, NH(CO)NHR⁵, NHCOOA, NA-acyl, NHCOOCH₂RS, NHSO₂CH₂W, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,

R⁸ is a 5-7-membered heterocyclic radical having 1-4 N, O and/or S atoms or is

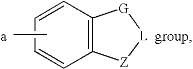

G and Z are each, independently of one another, —CH═, N, O or S,

L is —CH═, —CH═CH— or —CH₂—CH₂—CH₂—,

Hal is fluorine, chlorine, bromine or iodine,

n is 0, 1 or 2, and

m is 1 or 2,

or a tautomeric cyclised form, and the (E)-isomers and the salts of all isomers;

m) the compounds of the formula I described in WO 9841515

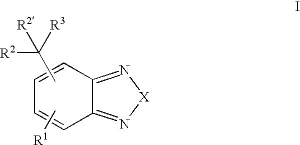

in which

X is O or S,

R¹ is H, Hal, OH, OA, A, NO₂, NH₂, NHA, NAA′, NHCOR⁴, NHCOR⁶, NHSO₂R⁴, NHSO₂R⁶, S(O)rnR⁶, SO₃H, SO₂NR⁴R⁴ or formyl,

R² and R^{2′} are each, independently of one another, A, (CH₂)ₘAr, (CH₂)ₙHet, CH₂COAr, CH₂COHet or OAr,

R² is additionally also H,

R³ is COOR⁴, CN, 1H-tetrazol-5-yl or CONHSO₂R⁵,

R⁴ and R^{4′} are each, independently of one another, H or A,

R⁵ is A or Ar,

R⁶ is phenyl or naphthyl, each of which is unsubstituted or monosubstituted, disubstituted or trisubstituted by A, NH₂, NHA, NM′, NO₂, CN or Hal,

R⁷ and R^{7′} are each, independently of one another, H or alkyl having 1-6 carbon atoms,

A and A′ are each, independently of one another, alkyl having 1-6 carbon atoms, in which one or two CH₂ groups may be replaced by O or S atoms or by —CR⁷═CR⁷′-groups and/or 1-7H atoms may be replaced by F, or benzyl,

Ar is phenyl or naphthyl, each of which is unsubstituted or monosubstituted, disubstituted or trisubstituted by A, OR⁴, NH₂, NHA, NA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂Rr, O(CH₂),ICOOR⁴, O(CH₂)OR⁴, SO₃H, SO₂NR⁴R⁴, S(O)ₘR⁶ or S(O)ₘR⁴,

Het is a monocyclic or bicyclic, saturated, unsaturated or aromatic heterocyclic radical having 1-4 N, O and/or S atoms, bonded via N or C, which may be unsubstituted or monosubstituted, disubstituted or trisubstituted by

Hal, A, R³, NH₂, NHA, NAA′, NO₂ and/or ═O,

Hal is fluorine, chlorine, bromine or iodine,

m is 0, 1 or 2, and

n is 1 or 2,

where, if R² is CH₂COAr and R^{2′} is H, R³ is not COOA, and salts thereof;

n) the compounds of the formula I described in WO 9841521

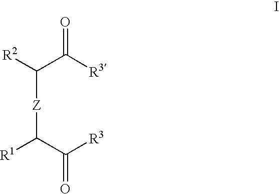

in which

Z is a single or double bond,

R¹ is a

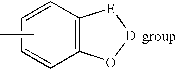

which is unsubstituted or monosubstituted in the phenyl part by R⁷, or is a

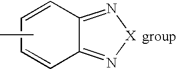

which is unsubstituted or monosubstituted in the cyclohexadienyl part by R⁷,

R² is A, Ar-(CH₂)ₘ, cycloalkyl-(CH₂)ₘ, Het-(CH₂)ₘ or R¹—(CHO₂)ₘ,

R³ and R^{3′} are each, independently of one another, OR⁴, NHSO₂R⁵, NH₂, NHA or NAA′,

R³ and R³ together are alternatively —O—, forming a cyclic anhydride,

R⁴ and R^{4′} are each, independently of one another, H or A,

R⁵ is A or Ar,

R⁶ is phenyl or naphthyl, each of which is unsubstituted or monosubstituted, disubstituted or trisubstituted by A, NH₂, NHA, NAA′, NO₂, CN or Hal,

R⁷ is A, COOR⁴, CN, 1H-tetrazol-5-yl, CONHSO₂R⁵, Hal, OR⁴, NO₂, NH₂, NHA, NAA′, NHCOR⁴, NHCOR⁶, NHSO₂R⁴, NHSO₂R⁶, S(O)ₖR⁴, S(O)ₖR^{4′}, SO₂NR⁴R^{4′} or formyl,

R⁸ and R^{8′} are each, independently of one another, H or alkyl having 1-6 carbon atoms,

E is CH₂ or O,

D is carbonyl or (CR⁴R^{4′})ₙ,

E and D together are alternatively CR⁴═R⁴,

X is S or O,

A and A′ are each, independently of one another, alkyl having 1-6 carbon atoms, in which one or two CH₂ groups may be replaced by O or S atoms or by —CR═CR⁸— groups and/or 1-7H atoms may be replaced by F, or benzyl,

Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHS₂R⁴, CONHSO₂R⁸, O(CH₂)ₙCOOR⁴, O(CHO₂OR⁴, SO₂NR⁴R^{4′}, S(O)IR⁶ or S(O)ₖR⁴,

Het is a monocyclic or bicyclic, saturated, unsaturated or aromatic heterocyclic radical having 1-4 N, O and/or S atoms, bonded via N or C, which may be unsubstituted, monosubstituted or disubstituted or trisubstituted by Hal, A, COOR⁴, CN, 1H-tetrazol-5-yl, CONHSO₂R⁵, NH₂, NHA, NAA′, NO₂ and/or ═O,

Hal is fluorine, chlorine, bromine or iodine,

k is 0, 1 or 2,

m is 0, 1 or 2, and

n is 1 or 2,

and the (Z)- and (E)-isomers and the salts of all isomers;

o) the compounds of the formula I described in WO 9842702

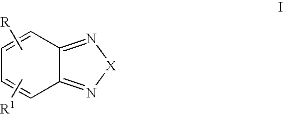

in which

R is

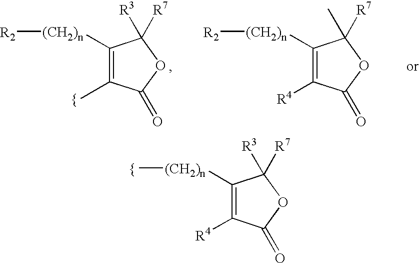

X and Y are each, independently of one another, O or S,

R¹ is H, Hal, OH, OA, A, alkylene-O-A, NO₂, NH₂, NH-acyl, SO₂NH₂, SO₂-A, SO₂NHA, CN or formyl,

R², R³

and R⁴ are each, independently of one another, a phenyl group which is unsubstituted or monosubstituted or polysubstituted by Hal, OH, OA, O-alkylene-R⁵, A, S-A, S-OA, SO₂A, S—OR⁵, SO₂Rr, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂,I-piperidinyl-CO—NH, 1-pyrrolidinyl-CONH, O(CH₂)ₙCOOA, O(CH₂)ₙCOOH, O(CH₂)ₙOH, O(CH₂),OA, CH₂OH, CH₂OA, COOH, COOA, CH₂COOH or CH₂COOA,

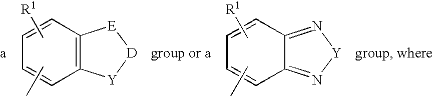

R² is additionally A or cycloalkyl,

R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂),COOA, O(CH₂),COOH, O(CH₂)ₙOH, O(CH₂)ₙOA, CH₂OH, CH₂OA, COOH, COOA, CH₂COOH or CH₂COOA,

A is alkyl having 1-6 carbon atoms, in which one or two CH₂ groups may be replaced by O or S atoms or by —CR⁸═CR⁶— groups and/or 1-7H atoms may be replaced by F,

D is carbonyl or IC(R⁶R))ₘ,

E is CH₂,S or O,

R⁶ and R^{6′} ar each, independently of one another, H, F or A,

R⁷ is —O—C(═Y)-NH—R⁸,

R⁸ is alkyl having 1-10 carbon atoms which is unsubstituted or monosubstituted or disubstituted by R⁸ and in which 1-2 carbon atoms may be replaced by 0 and/or S, and/or may be substituted by ═O, or cycloalkyl, in which 1-2 carbon atoms may be replaced by N, O and/or S,

R⁹ is phenyl which is unsubstituted or monosubstituted or disubstituted by Hal, or is naphthyl, A-O—C(═O)— or Hal,

Hal is fluorine, chlorine, bromine or iodine,

n is 0, 1 or 2, and

m is 1 or 2,

and salts thereof;

p) the compounds of the formula I described in WO 9842709

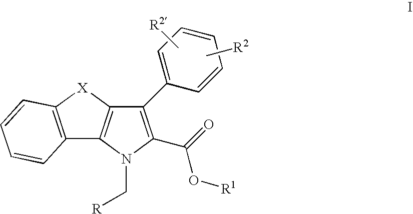

in which

X is N—R³, O or S,

R is 2,1,3-benzothiadiazol-4- or 5-yl or 2,1-benzoisothiazol-5- or 6-yl, each of which is unsubstituted or monosubstituted or disubstituted by R² and/or R²′, or phenyl which is unsubstituted or monosubstituted, disubstituted or trisubstituted by R² and/or R²′,

R¹ is H or A,

R² and R² are each, independently of one another, H, A, OH, OA, Hal, OCF₃, OCHF₂, —O—CO-A, —O-alkylene-COOR¹, —O-alkylene-CH₂—OR¹, or OCH₂-phenyl or —O—CO-phenyl, each of which is unsubstituted or monosubstituted or disubstituted in the phenyl part by R⁴ and/or R⁴′,

R² and R^{2′} together are alternatively —OCH₂O—, —OCH₂CH₂O— or —OCH₂CH₂—,

R³ is H, A, alkylene-O-A, —CO-OA, or alkylene-phenyl which is unsubstituted or mono-substituted or disubstituted in the phenyl part by R⁴ and/or R⁴′,

R⁴ and R^{4″} are each, independently of one another, H, A, OH, OA, Hal, COOR¹ or CH₂OR¹,

A is alkyl having 1-6 carbon atoms,

Hal is fluorine, chlorine, bromine or iodine,

and their salts;

q) the compounds of the formula I described in WO 9905132

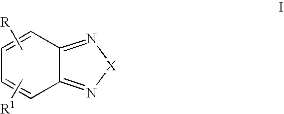

in which

R is

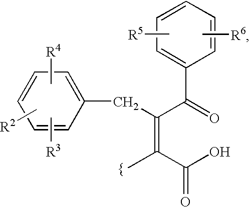

X is O or S,

R¹ is H, Hal, OA or A,

R², R³, R⁵ _{and R}⁶ are each, independently of one another, H, Hal, A, OA or R⁴,

R⁴ is —O—(CH₂)ₙ-Cy,

Cy is cycloalkyl having 3-8 carbon atoms,

A is alkyl having 1-6 carbon atoms, in which one or two CH₂ groups may be replaced by O or S atoms or by —CR⁵═CR⁶— groups and/or 1-7H atoms may be replaced by F,

R⁵ and R^{5′} are each, independently of one another, H, F or A,

Hal is fluorine, chlorine, bromine or iodine,

n is 0, 1 or 2,

or a tautomeric cyclised form, and the (E)-isomers and the salts of all isomers.

The phosphodiesterase V inhibitors of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

In the compounds of the formulae II or III, R¹, R², R³, R⁴ and X have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, furthermore also 2-naphthalenesulfonyloxy).

The compounds of the formula I can preferably be obtained by reacting compounds of the formula II with compounds of the formula III.

If desired, the starting materials can also be formed in situ by not isolating them from the reaction mixture, but instead immediately converting them further into the compounds of the formula I.

On the other hand, it is possible to carry out the reaction stepwise.

The starting compounds of the formulae II and III are generally known. If they are not known, they can be prepared by methods known per se. Compounds of the formula II can be prepared by methods known from the literature, for example from 4-amino-3-alkoxycarbonylpyrazoles by cyclisation using nitrites followed by reaction of the cyclisation products with phosphorus oxychloride (analogously to Houben Weyl E9b/2).

In detail, the reaction of the compounds of the formula II with the compounds of the formula III is carried out in the presence or absence of an inert solvent at temperatures between about −20 and about 150°, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline, or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of the said solvents.

It is furthermore possible to convert a radical X in a compound of the formula I into another radical X, for example by hydrolysing an ester or a cyano group to give a COOH group.

Ester groups can be saponified, for example, using NAOH or KOH in water, water/THF or water/dioxane at temperatures between 0 and 100°. Carboxylic acids can be converted into the corresponding carbonyl chlorides, for example using thionyl chloride, and these can be converted into carboxamides. Elimination of water therefrom in a known manner gives carbonitriles.

An acid of the formula I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I can be converted into the corresponding metal salt, in particular alkali metal or alkaline earth metal salt, or into the corresponding ammonium salt using a base (for example sodium hydroxide, potassium hydroxide, sodium carbonate or potassium carbonate). Also suitable for this reaction are, in particular, organic bases which give physiologically acceptable salts, such as, for example, ethanolamine.

On the other hand, a base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, or laurylsulfuric acid. Salts with physiologically unacceptable acids, for example picrates, can be used for the isolation and/or purification of the compounds of the formula I.

The invention furthermore relates to the pharmaceutical formulations comprising at least one phosphodiesterase V inhibitor of the formula I and/or one of its physiologically acceptable salts and at least one endothelin receptor antagonist and comprising one or more excipients and/or assistants.

The pharmaceutical preparations are prepared, in particular, by non-chemical methods, in which the active ingredients are converted into a suitable dosage form together with at least one solid, liquid and/or semi-liquid excipient or assistant.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants, flavours and/or a plurality of further active ingredients, for example one or more vitamins. They can furthermore be administered as nasal sprays.

In general, the substances are preferably administered in doses of between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention therefore also relates to the use of the pharmaceutical preparations described for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

The invention relates, in particular, to the use of the formulations according to the invention for the preparation of a medicament for the treatment of high pulmonary pressure, congestive heart failure (CHF), chronic obstructive pulmonary disease (COPD), cor pulmonale and/or dextrocardiac insufficiency.

The constituents of the novel pharmaceutical [preparation are preferably administered combined. However, they can also be administered individually at the same time or successively.

The invention also relates to a set (kit) consisting of separate packs of

(a) an effective amount of [7-(3-chloro-4-methoxybenzylamino)-1-methyl-3-propyl-1H-pyrazolo[4,3-d]pyrimidin-5-ylmethoxy]acetic acid and/or physiologically acceptable salts and/or solvates thereof and

(b) an effective amount of an endothelin receptor antagonist.

The set comprises suitable containers, such as boxes, individual bottles, cartons, bags or ampoules. The set may comprise, for example, separate ampoules each containing an effective amount of [7-(3-chloro-4-methoxybenzylamino)-1-methyl-3-propyl-1H-pyrazolo[4,3-d]pyrimidin-5-ylmethoxy]-acetic acid, and/or physiologically acceptable salts and/or solvates thereof and of the endothelin receptor antagonist in dissolved or lyophilised form.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS):

EI (electron impact ionisation) M⁺

FAB (fast atom bombardment) (M+H)+

### EXAMPLE 1

3 g of methyl 3-[7-chloro-1-methyl-3-propyl-1H-pyrazolo[4,3-d)pyrimidin-5-yl]propionate and 1.9 g of 3-chloro-4-methoxybenzylamine (“A”) in 50 ml of dimethylformamide (DMF) is stirred for 12 hours at 60° in the presence of potassium carbonate. After filtration, the solvent is removed, and the product is subjected to conventional work-up, giving 4.6 g of methyl 3-[7-(3-chloro-4-methoxybenzylamino)-1-methyl-3-propyl-1H-pyrazolo[4,3-d]-pyrimidin-5-yl]propionate as a colourless oil.

Analogous Reaction of “A”

with methyl 2-[7-chloro-1-methyl-3-propyl-1H-pyrazolo[4,3-d]pyrimidin-5-yl]acetate gives methyl 2-[7-(3-chloro-4-methoxybenzylamino)-1-methyl-3-propyl-1H-pyrazolo[4,3-d]pyrimidin-5-yl]acetate.

Analogous Reaction of 3,4-methylenedioxybenzylamine with methyl 3-[7-chloro-1-methyl-3-propyl-1H-pyrazolo[4,3-d]pyrimidin-5-yl]propionate gives

methyl 3-[7-(3,4-methylenedioxybenzylamino)-1-methyl-3-propyl-1H-pyrazolo[4,3-d]pyrimidin-5-yl]propionate.

Analogous Reaction of “A”

with methyl 4-[7-chloro-1-methyl-3-propyl-1H-pyrazolo[4,3-d]pyrimidin-5-yl]butyrate gives methyl 4-[7-(3-chloro-4-methoxybenzylamino)-1-methyl-3-propyl-1H-pyrazolo[4,3-d]pyrimidin-5-yl]butyrate.

Analogous Reaction of 3,4-methylenedioxybenzylamine

with methyl 4-[7-chloro-1-methyl-3-propyl-1H-pyrazolo[4,3-d]pyrimidin-5-yl]butyrate gives

methyl 4-[-(3,4-methylenedioxybenzylamino)-1-methyl-3-propyl-1H-pyrazolo[4,3-d]pyrimidin-5-yl]butyrate.

Analogous Reaction of “A”

with methyl 5-[7-chloro-1-methyl-3-propyl-1H-pyrazolo[4,3-d]pyrimidin-5-yl]valerate gives

methyl 5-[7-(3-chloro-4-methoxybenzylamino)-1-methyl-3-propyl-1H-pyrazolo[4,3-d]pyrimidin-5-yl]valerate.

Analogous Reaction of 3,4-methylenedioxybenzylamine

with methyl 5-[7-chloro-1-methyl-3-propyl-1H-pyrazolo[4,3-d]pyrimidin-5-yl]valerate gives

methyl 5-[7-(3,4-methylenedioxybenzylamino)-1-methyl-3-propyl-1H-pyrazolo[4,3-d]pyrimidin-5-yl]valerate.

Analogous Reaction of “A”

with methyl 7-[7-chloro-1-methyl-3-propyl-1H-pyrazolo[4,3-d]pyrimidin-5-yl]heptanoate gives

methyl 7-[7-(3-chloro-4-methoxybenzylamino)-1-methyl-3-propyl-1H-pyrazolo[4,3-d]pyrimidin-5-yl]heptanoate.

Analogous Reaction of 3,4-methylenedioxybenzylamine

with methyl 7-[7-chloro-1-methyl-3-propyl-1H-pyrazolo[4,3-d]pyrimidin-5-yl]heptanoate gives

methyl 7-[7-(3,4-methylenedioxybenzylamino)-1-methyl-3-propyl-1H-pyrazolo[4,3-d]pyrimidin-5-yl]heptanoate.

Analogous Reaction of “A”

with methyl 2-[4-(7-chloro-1-methyl-3-propyl-1H-pyrazolo[4,3-d]pyrimidin-5-yl)-cyclohex-1-yl]acetate gives

methyl 24-[7-(3-chloro-4-methoxybenzylamino)-1-methyl-3-propyl-1H-pyrazolo[4,3-d]pyrimidin-5-yl]cyclohexyl-1-yl]acetate.

Analogous Reaction of 3,4-methylenedioxybenzylamine

with methyl 2-[4-(7-chloro-1-methyl-3-propy[-1H-pyrazoto[4,3-d]pyrimidin-5-yl)-cyclohex-1-yl]acetate gives

methyl 2-{4-[7-(3,4-methylenedioxybenzylamino)-1-methyl-3-propyl-1H-pyrazolo[4,3-d]pyrimidin-5-yl]cyclohexyl-1-yl}acetate.

Analogous Reaction of Benzylamine

with methyl 3-[7-chloro-1-methyl-3-propyl-1H-pyrazolo[4,3-d]pyrimidin-5-yl]propionate gives

methyl 3-[7-benzylamino-1-methyl-3-propyl-1H-pyrazolo[4,3-d]-pyrimidin-5-yl]propionate;

with methyl 4-[7-chloro-1-methyl-3-propyl-1H-pyrazolo[4,3-d]pyrimidin-5-yl]butyrate gives

methyl 4-[7-benzylamino-1-methyl-3-propyl-1H-pyrazolo[4,3-d]-pyrimidin-5-yl]butyrate;

with methyl 5-V7-chloro-1-methyl-3-propyl-1H-pyrazolo[4,3-d]pyrimidin-5-yl]valerate gives

methyl 5-[7-benzylamino-1-methyl-3-propyl-1H-pyrazolo[4,3-d]-pyrimidin-5-yl]valerate.

Analogous Reaction of “A”

with methyl 4-[7-chloro-1-methyl-3-propyl-1H-pyrazolo[4,3-d]pyrimidin-5-yl]cyclohexanecarboxylate gives

methyl 4-[7-(3-chloro-4-methoxybenzylamino)-1-methyl-3-propyl-1H-pyrazolo[4,3-d]pyrimidin-5-yl]cyclohexanecarboxylate

and reaction of 3,4-methylenedioxybenzylamine gives

methyl 4-[7-(3,4-methylenedioxybenzylamino)-1-methyl-3-propyl-1H-pyrazolo[4,3-d]pyrimidin-5-yl]cyclohexancarboxylates.

### EXAMPLE 2

4.3 g of methyl 3-(7-(3-chloro-4-methoxybenzy(amino)-1-methyl-3-propyl-1H-pyrazolo[4,3-d]pyrimidin-5-yl]propionate are dissolved in 30 ml of tetrahydrofuran (THF), 10 ml of 10% NaOH are added, and the mixture is stirred at 60° for 8 hours. After 10% HCl has been added, the precipitated crystals are separated off and recrystallised from methanol, giving 3.7 g of 3-[7-(3-chloro-4-methoxybenzylamino)-1-methyl-3-propyl-1H-pyrazolo-[4,3-d]pyrimidin-5-yl]propionic acid, m.p. 178°.

Evaporation with the equivalent amount of methanolic potassium hydroxide solution gives the potassium salt of the acid as an amorphous powder.

Analogous reaction of the esters listed in Example 1 gives the following compounds:

2-[7-(3-chloro-4-methoxybenzylamino)-1-methyl-3-propyl-1H-pyrazolo[4,3-d]pyrimidin-5-yl]acetic acid,

[3-[7-(3,4-methylenedioxybenzylamino)-1-methyl-3-propyl-1H-pyrazolo[4,3-d]pyrimidin-5-yl]propionic acid,

4-[7-(3-chloro-4-methoxybenzylamino)-1-methyl-3-propyl-1H-pyrazolo[4,3-d]pyrimidin-5-yl]butyric acid, m.p. 152°;

4-[7-(3,4-methylenedioxybenzylamino)-1-methyl-3-propyl-1H-pyrazolo[4,3-d]pyrimidin-5-yl]butyric acid, m.p. 172°;

5-[7-(3-chloro-4-methoxybenzylamino)-1-methyl-3-propyl-1H-pyrazolo[4,3-d]pyrimidin-5-yl]valeric acid, m.p. 159°;

5-[7-(3,4-methylenedioxybenzylamino)-1-methyl-3-propyl-1H-pyrazolo[4,3-d]pyrimidin-5-yl]valeric acid, ethanolamine salt, m.p. 160°;

7-[7-(3-chloro-4-methoxybenzylamino)-1-methyl-3-propyl-1H-pyrazolo[4,3-d]pyrimidin-5-yl]heptanoic acid,

7-[7-(3,4-methylenedioxybenzylamino)-1-methyl-3-propyl-1H-pyrazolo[4,3-d]pyrimidin-5-yl]heptanoic acid,

2-{4-[7-(3-chloro-4-methoxybenzylamino)-1-methyl-3-propyl-1H-pyrazolo[4,3-d]pyrimidin-5-yl]cyclohexyl-1-yl}acetic acid,

2-{4-[7-(3,4-methylenedioxybenzylamino)-1-methyl-3-propyl-1H-pyrazolo[4,3-d]pyrimidin-5-yl]cyclohexyl-1-yl}acetic acid,

3-[7-benzylamino-1-methyl-3-propyl-1H-pyrazolo[4,3-d]pyrimidin-5-yl]-propionic acid,

4-[7-benzylamino-1-methyl-3-propyl-1H-pyrazolo[4,3-d]pyrimidin-5-yl]butyric acid,

5-(7-benzylamino-1-methyl-3-propyl-1H-pyrazolo[4,3-d]pyrimidin-5-yl]valeric acid, m.p. 1850;

4-[7-(3-chloro-4-methoxybenzylamino)-1-methyl-3-propyl-1H-pyrazolo[4,3-d]pyrimidin-5-yl]cyclohexanecarboxylic acid, 4-[7-(3,4-methylenedioxybenzylamino)-1-methyl-3-propyl-1H-pyrazolo[4,3-d]pyrimidin-5-yl]cyclohexanecarboxylic acid.

The following compounds are obtained analogously:

5-[7-(3-chloro-4-methoxybenzylamino)-1-methyl-3-isopropyl-1H-pyrazolo[4,3-d]pyrimidin-5-yl]valeric acid, cyclohexylamine salt, m.p. 1480;

4-[7-(3-chloro-4-methoxybenzylamino)-1-methyl-3-ethyl-1H-pyrazolo[4,3-d]pyrimidin-5-yl]butyric acid, m.p. 176°;

4-[7-(3,4-methylenedioxybenzylamino)-1-methyl-3-ethyl-1H-pyrazolo[4,3-d]pyrimidin-5-yl]butyric acid, m.p. 187°;

4-[7-(3-chloro-4-methoxybenzylamino)-1-ethyl-3-methyl-1H-pyrazolo[4,3-d]pyrimidin-5-yl]butyric acid, m.p. 206°;

4-[7-(3,4-methylenedioxybenzylamino)-1-ethyl-3-methyl-1H-pyrazolo[4,3-d]pyrimidin-5-yl]butyric acid, m.p. 1770;

4-[7-benzylamino-1-methyl-3-ethyl-1H-pyrazolo[4,3-d]pyrimidin-5-yl]butyric acid, m.p. 208°;

4-[7-(3-chloro-4-methoxybenzylamino)-1-methyl-3-methyl-1H-pyrazolo[4,3]pyrimidin-5-yl)butyric acid, m.p. 250°;

4-[7-(3,4-methylenedioxybenzylamino)-1-methyl-3-methyl-1H-pyrazoio[4,3-d]pyrimidin-5-yl]butyric acid, m.p. 225°;

4-[7-benzylamino-1-methyl-3-methyl-1H-pyrazolo[4,3-d]pyrimidin-5-yl]butyric acid, m.p. 201°;

5-[7-(4-methoxybenzylamino)-1-methyl-3-propyl-1H-pyrazolo[4,3-d]-pyrimidin-5-yl[valeric acid, m.p. 160°;

5-[7-(3-methoxybenzylamino)-1-methyl-3-propyl-1H-pyrazolo[4,3-d-]pyrimidin-5-yl]valeric acid, m.p. 141°;

5-[7-(4-chlorobenzylamino)-1-methyl-3-propyl-1H-pyrazolo[4,3-d]-pyrimidin-5-yl]valeric acid, m.p. 148°;

5-[7-(3-chlorobenzylamino)-1-methyl-3-propyl-1H-pyrazolo[4, 3d]-pyrimidin-5-yl]valeric acid, m.p. 151°;

### EXAMPLE 3

A mixture of 1.8 g of methyl 4-[7-chloro-1-methyl-3-propyl-1H-pyrazolo-[4,3-d]pyrimidin-5-yl]phenylcarboxylate (“B”) and 1.5 g of 3-chloro-4-methoxybenzylamine in 20 ml of N-methylpyrrolidone is heated at 110° for 4 hours. After cooling, the mixture is subjected to conventional work-up, giving 2.2 g of methyl 4-[7-(3-chloro-4-methoxybenzylamino]-methyl-3-propyl-1H-pyrazolo[4,3-d]pyrimidin-5-yl]benzoate.

Analogously to Example 2, 1.2 g of the ester give 1.0 g of

4-[7-(3-chloro-4-methoxybenzylamino]-methyl-3-propyl-1H-pyrazolo-[4,3-d]pyrimidin-5-yl]benzoic acid, ethanolamine salt, m.p. 139°.

Analogously to Example 1, “B” and 3,4-methylenedioxybenzylamine give

methyl 4-[7-(3,4-methylenedioxybenzylamino)-1-methyl-3-propyl-1H-pyrazolo[4,3-d]pyrimidin-5-yl]benzoate, and ester hydrolysis thereof gives

4-[7-(3,4-methylenedioxybenzylamino)-1-methyl-3-propyl-1H-pyrazolo[4,3-d]pyrimidin-5-yl]benzoic acid.

The following compounds are obtained analogously:

4-[7-(3-chloro-4-methoxybenzylamino)-1-methyl-3-propyl-1H-pyrazolo[4,3-d]pyrimidin-5-yl]phenylacetic acid, glucamine salt, m.p. 114°

and

4-17-(3,4-methylenedioxybenzylamino)-1-methyl-3-propyl-1H-pyrazolo[4,3-d]pyrimidin-5-yl]phenylacetic acid.

### EXAMPLE 4

1 equivalent of 3-[7-(3-chloro-4-methoxybenzylamino)-1-methyl-3-propyl-1H-pyrazolo[4,3-d]pyrimidin-5-yl]propionic acid and 1.2 equivalents of thionyl chloride are stirred in dichloromethane for 2 hours. The solvent is removed, giving 3-[7-(3chloro-4-methoxybenzylamino)-1-methyl-3-propyl-1H-pyrazolo[4,3-d]pyrimidin-5-yl]propionyl chloride. The product is transferred into aqueous ammonia, and the mixture is stirred for one hour and subjected to conventional work-up, giving 3-[7-(3-chloro-4-methoxybenzylamino)-1-methyl-3-propyl-1 H-pyrazolo[4,3-d]-pyrimidin-5-yl]propionamide.

### EXAMPLE 5

1 equivalent of DMF and 1 equivalent of oxalyl chloride are dissolved in acetonitrile at 00.1 equivalent of 3-[7-(3-chloro-4-methoxybenzylamino)-1-methyl-3-propyl-1H-pyrazolo[4,3-d]pyrimidin-5-yl]propionamide is then added. The mixture is stirred for a further one hour. Conventional work-up gives 3-[7-(3-chloro-4-methoxybenzylamino)-1-methyl-3-propyl-1H-pyrazolo[4,3-d]pyrimidin-5-yl]propionitrile.

### EXAMPLE 6

Analogously to Examples 1, 2 and 3, reaction of the corresponding chloro-pyrimidine derivatives with 3,4-ethylenedioxybenzylamine gives the following carboxylic acids:

4-[7-(3,4-ethylenedioxybenzylamino)-1-methyl-3-propyl-1H-pyrazolo-[4,3-d]pyrimidin-5-yl]butyric acid,

3-[7-(3,4-ethylenedioxybenzylamino)-1-methyl-3-propyl-1H-pyrazolo-[4,3-d]pyrimidin-5-yl]propionic acid,

5-[7-(3,4-ethylenedioxybenzylamino)-1-methyl-3-propyl-1H-pyrazolo-[4,3-d]pyrimidin-5-yl]valeric acid,

7-[7-(3,4-ethylenedioxybenzylamino)-1-methyl-3-propyl-1H-pyrazolo-[4,3-d]pyrimidin-5-yl)heptanoic acid,

2-{4-[7-(3,4-ethylenedioxybenzylamino)-1-methyl-3-propyl-1H-pyrazoio[4,3-d]pyrimidin-5-yl]cyclohexyl-1-yl}acetic acid,

4-[7-(3,4-ethylenedioxybenzylamino)-1-methyl-3-propyl-1H-pyrazolo-[4,3-d]pyrimidin-5-yl]cyclohexanecarboxylic acid,

4-[7-(3,4-ethylenedioxybenzylamino)-1-methyl-3-propyl-1H-pyrazoio-[4,3-d]pyrimidin-5-yl]benzoic acid,

4-[7-(3,4-ethylenedioxybenzylamino)-1-methyl-3-propyl-1H-pyrazolo-(4,3-pyrimidin-5-yl]benzoic acid,

4-[7-(3,4-ethylenedioxybenzylamino)-1-methyl-3-propyl-1H-pyrazolo-[4,3-d]pyrimidin-5-yl]phenylacetic acid.

Analogous reaction with 3,4-dichlorobenzylamine gives the following compounds:

4-[7-(3,4-dichlorobenzylamino)-1-methyl-3-propyl-1H-pyrazolo-[4,3-d]pyrimidin-5-yl]butyric acid, m.p. 2090;

3-[7-(3,4-dichlorobenzylamino)-1-methyl-3-propyl-1H-pyrazolo-[4,3-d]pyrimidin-5-yl]propionic acid,

5-(7-(3,4-dichlorobenzylamino)-1-methyl-3-propyl-1H-pyrazolo-[4,3-d]pyrimidin-5-yl]valeric acid,

7-[7-(3,4-dichlorobenzylamino)-1-methyl-3-propyl-1H-pyrazolo-[4,3-d]pyrimidin-5-yl]heptanoic acid,

2-{4-[7-(3,4-dichlorobenzylamino)-1-methyl-3-propyl-1H-pyrazolo-[4,3-d]pyrimidin-5-yl]cyclohexyl-1-yl)acetic acid,

4-[7-(3,4-dichlorobenzylamino)-1-methyl-3-propyl-1H-pyrazolo-[4,3-d]pyrimidin-5-yl]cyclohexanecarboxylic acid,

4-[7-(3,4-dichlorobenzylamino)-1-methyl-3-propyl-1H-pyrazolo-[4,3-d]pyrimidin-5-yl]benzoic acid,

4-[7-(3,4-dichlorobenzylamino)-1-methyl-3-propyl-1H-pyrazolo-[4,3-d]pyrimidin-5-yl]phenylacetic acid.

Analogous reaction with 3-chloro-4-ethoxybenzylamine gives the following compounds:

4-[7-(3-chloro-4-ethoxybenzylamino)-1-methyl-3-propyl-1H-pyrazolo-[4,3-d]pyrimidin-5-yl]butyric acid,

3-[7-(3-chloro-4-ethoxybenzylamino)-1-methyl-3-propyl-1H-pyrazolo-[4,3-d]pyrimidin-5-yl]propionic acid,

5-[7-(3-chloro-4-ethoxybenzylamino)-1-methyl-3-propyl-1H-pyrazolo-[4,3-d]pyrimidin-5-yl]valeric acid,

7-[7-(3-chloro-4-ethoxybenzylamino)-1-methyl-3-propyl-1H-pyrazolo-[4,3-d]pyrimidin-5-yl]heptanoic acid,

2-{4-[7-(3-chloro-4-ethoxybenzylamino)-1-methyl-3-propyl-1H-pyrazolo[4,3-d]pyrimidin-5-yl]cyclohexyl-1-yl}acetic acid,

4-[7-(3-chloro-4-ethoxybenzy(amino)-1-methyl-3-propyl- H-pyrazolo-[4,3-d]pyrimidin-5-yl]cyclohexanecarboxylic acid,

4-[7-(3-chloro-4-ethoxybenzylamino)-1-methyl-3-propyl-1H-pyrazolo[4,3-d]pyrimidin-5-yl]benzoic acid,

4-[7-(3-chloro-4-ethoxybenzylamino)-1-methyl-3-propyl-1H-pyrazolo[4,3-d]pyrimidin-5-yl]phenylacetic acid.

Analogous reaction with 3-chloro-4-isopropoxybenzylamine gives the following compounds

4-[7-(3-chloro-4-isopropoxybenzylamino)-1-methyl-3-propyl-1H-pyrazolo[4,3-d]pyrimidin-5-yl]butyric acid,

3-[7-(3-chloro-4-isopropoxybenzylamino)-1-methyl-3-propyl-1H-pyrazolo[4,3d]pyrimidin-5-yl]propionic acid,

5-[7-(3-chloro-4-isopropoxybenzylamino)-1-methyl-3-propyl-1H-pyrazolo[4,3-d]pyrimidin-5-yl]valeric acid,

7-[7-(3-chloro-4-isopropoxybenzylamino)-1-methyl-3-propyl-1H-pyrazolo[4,3-d]pyrimidin-5-yl]heptanoic acid,

2-(4-[7-(3-chloro-4-isopropoxybenzylamino)-1-methyl-3-propyl-1H-pyrazolo[4,3-d]pyrimidin-5-yl]cyclohexyl-1-yl}acetic acid,

4-[7-(3-chloro-4-isopropoxybenzylamino)-1-methyl-3-propyl-1H-pyrazolo[4,3-d]pyrimidin-5-yl]cyclohexanecarboxylic acid,

4-[7-(3-chloro-4-isopropoxybenzylamino)-1-methyl-3-propyl-1H-pyrazolo[4,3-d]pyrimidin-5-yl]benzoic acid,

4-[7-(3-chloro-4-isopropoxybenzylamino)-1-methyl-3-propyl-1H-pyrazolo[4,3-d]pyrimidin-5-yl]phenylacetic acid.

### EXAMPLE 7

The following compound is obtained analogously to Examples 1 and 2:

[7-(3-chloro-4-methoxybenzylamino)-1-methyl-3-propyl-1H-pyrazofo[4,3-d]-pyrimidin-5-ylmethoxy]acetic acid, ethanolamine salt, m.p. 138°.

The examples below relate to pharmaceutical preparations:

### EXAMPLE A

### Injection Vials

A solution of 100 g of an active ingredient of the formula I , 100 g of the endothelin receptor antagonist and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B

### Suppositories

A mixture of 20 g of an active ingredient of the formula I and 20 g of an endothelin receptor antagonist is melted with 100 g of soya lecithin and 1400 g of cocoa butter, poured into moulds and allowed to cool. Each suppository contains 20 mg of each active ingredient.

### EXAMPLE C

### Solution

A solution is prepared from 1 g of an active ingredient of the formula I , 1 g of an endothelin receptor antagonist, 9.38 g of NaH₂PO₄.2H₂O, 28.489 of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula 1 and 500 mg of an endothelin receptor antagonist are mixed with 99.59 of Vaseline under aseptic conditions.

### EXAMPLE E:

### Tablets

A mixture of 1 kg of active ingredient of the formula I , 1 g of an endothelin receptor antagonist, 4 kg of lactose, 1.2 kg of potato starch, 0.2 kg of talc and 0.1 kg of magnesium stearate is pressed in a conventional manner to give tablets in such a way that each tablet contains 10 mg of each active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula 1 and 2 kg of an endothelin receptor antagonist are introduced in a conventional manner into hard gelatine capsules in such a way that each capsule contains 20 mg of each active ingredient.

### EXAMPLE H

### Ampoules

A solution of 1 kg of active ingredient of the formula 1 and 1 kg of an endothelin receptor antagonist in 60 l of bidistilled water is sterile filtered, transferred into ampoules, lyophilised under sterile conditions and sealed under sterile conditions. Each ampoule contains 10 mg of each active ingredient.

### EXAMPLE I

### Inhalation Spray

14 g of active ingredient of the formula 1 and 14 g of an endothelin receptor antagonist are dissolved in 10 l of isotonic NaCl solution, and the solution is transferred into commercially available spray containers with pump mechanism. The solution can be sprayed into the mouth or nose. One spray shot (about 0.1 ml) corresponds to a dose of about 0.14 mg of each active ingredient.

Comp unds of th Formula I-I:

The compounds of the formula I-I according to claim 1 and their salts are prepared by a process,

characterised in that

a) a compound of the formula II-1

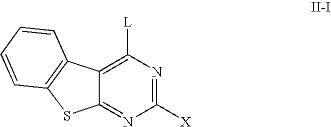

in which

X is as defined above,

and L is Cl, Br, OH, SCH₃ or a reactive esterified OH group,

is reacted with a compound of the formula III

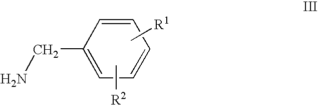

in which

R¹ and R² are as defined above, or

b) a radical X in a compound of the formula I-I is converted into another radical X by, for example, hydrolysing an ester group to a COOH group or converting a COOH group into an amide or into a cyano group, and/or in that a compound of the formula I-1 is converted into one of its salts.

The term solvates of the compounds of the formula I-I is taken to mean adductions of inert solvent molecules onto the compounds of the formula I-II which form owing to their mutual attractive forces. Solvates are, for example, mono- or dihydrates or alcoholates.

Above and below, the radicals R¹, R², R³, R⁴, R⁵, R⁶, R⁷, X and L are as defined for the formulae I-I, II-I and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁴, R⁵ or R⁶ radical which is monosubstituted by R⁷.

R⁴ is a linear or branched alkylene radical having 1-10 carbon atoms, where the alkylene radical is preferably, for example, methylene, ethylene, propylene, isopropylene, butylene, isobutylene, sec-butylene, pentylene, 1-2- or 3-methylbutylene, 1,1-, 1,2- or 2,2-dimethylpropylene, 1-ethyl-propylene, hexylene, 1-, 2-, 3- or 4-methylpentylene, 1,1-, 1,2-, 1,3-, 2,2-, 2,3- or 3,3-dimethylbutylene, 1- or 2ethylbutylene, 1-ethyl-1-methyl-propylene, 1-ethyl-2-methylpropylene, 1,1,2- or 1,2,2-trimethylpropylene, linear or branched heptylene, octylene, nonylene or decylene.

R⁵ is furthermore, for example, but-2-enylene or hex-3-enylene. Very particular preference is given to ethylene, propylene or butylene.

R⁵ is cycloalkylalkylene having 5-12 carbon atoms, preferably, for example, cyclopentylmethylene, cyclohexylmethylene, cyclohexylethylene, cyclo-hexylpropylene or cyclohexylbutylene. R⁵ is alternatively cycloalkyl, preferably having 5-7 carbon atoms. Cycloalkyl is, for example, cyclopentyl, cyclohexyl or cycloheptyl.

Hal is preferably F, Cl or Br, but alternatively I.

The radicals R¹ and R² may be identical or different and are preferably in the 3- or 4-position of the phenyl ring. They are, for example, each, independently of one another, H, hydroxyl, alkyl, F, Cl, Br or I or together are alkylene, such as, for example, propylene, butylene or pentylene, furthermore ethyleneoxy, methylenedioxy or ethylenedioxy. They are alternatively preferably each alkoxy, such as, for example, methoxy, ethoxy or propoxy.

The radical R⁷ is preferably, for example, COOH, COOCH₃, COOC₂H₆, CONH₂, —CON(CH₃)₂, CONHCH₃ or CN.

Throughout the invention, all radicals which occur more than once may be identical or different, i.e. are independent of one another.

The invention relates, in particular, to pharmaceutical formulations comprising an endothelin receptor antagonist and at least one compound of the formula I-I in which at least one of the said radicals has one of the preferred meanings indicated above. Some preferred groups of compounds may be expressed by the following sub-formulae Ia to Ie, which conform to the formula I-II and in which the radicals not designated in greater detail are as defined for the formula I-I, but in which

in Ia

X is R⁴, phenyl or phenylmethyl, each of which is substituted by COOH, COOA, CONH₂, CONA₂, CONHA or CN;

in Ib

R¹ and R² together are alkylene having 3-5 carbon atoms, —O—CH₂—CH₂—, —O—CH₂—O— or —O—CH₂—CH₂—O—,

X is R⁴, phenyl or phenylmethyl, each of which is substituted by COOH, COOA, CONH₂, CONH₂, CONHA or CN;

in Ic

R¹ and R² are each, independently of one another, H, A, OA or Hal,

R¹ and R² together are alkylene having 3-5 carbon atoms, —O—CH₂—CH₂—, —O—CH₂—O— or —O—CH₂—CH₂—O—,

X is R⁴, phenyl or phenylmethyl, each of which is substituted by COOH, COOA, CONH₂, CONA₂, CONHA or CN;

in Id

R¹ and R² are each, independently of one another, H, A, OA or Hal,

R¹ and R² together are alternatively alkylene having 3-5 carbon atoms, —O—CH₂—CH₂—, —O—CH₂—O— or —O—CH₂CH₂—O—,

X is alkylene having 2-5 carbon atoms, cyclohexyl, phenyl or phenylmethyl, each of which is mono-substituted by R¹,

R⁷ is COOH or COOA,

A is alkyl having from 1 to 6 carbon atoms,

Hal is F, Cl, Br or I;

in Ie

R¹ and R² are each, independently of one another, H, A, OA or Ha),

R¹ and R² together are alkylene having 3-5 carbon atoms, —O—CH₂—CH₂—, —O—CH₂—O— or —O—CH₂—CH₂—O—,

X is alkylene having 2-5 carbon atoms, cyclohexyl, phenyl or phenylmethyl, each of which is mono-substituted by R¹,

R⁷ is COOH or COOA,

A is alkyl having from 1 to 6 carbon atoms,

Hal is F, Cl, Br or 1. The invention preferably relates to a formulation comprising [4-[4-(3-chloro-4-methoxybenzylamino)benzothieno[2,3-d]pyrimidin-2-yl]cyclohexane-carboxylic acid and physiologically acceptable salts and/or solvates thereof and at least one endothelin receptor antagonist.

Besides the free acid, the ethanolamine salt is preferred.

Preferred endothelin receptor antagonists are those listed above under the PDE V inhibitors of the formula I.

The phosphodiesterase V inhibitors of the formula I-I and also the starting materials for their preparation are, in addition, prepared by methods known per se, as described in the literature (for example in the standard works, such as Houben-Weyl, Methoden der organischen Chemie [Methods of Organic Chemistry), Georg-Thieme-Verlag, Stuttgart), to be precise under reaction conditions which are known and suitable for the said reactions. Use can also be made here of variants which are known per se, but are not mentioned here in greater detail.

In the compounds of the formulae II-I or III, R¹, R², R³, R⁴, X and n have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, furthermore also 2-naphthalenesulfonyloxy).

The compounds of the formula I-I can preferably be obtained by a process in which compounds of the formula II-I are reacted with compounds of the formula III.

If desired, the starting materials can also be formed in situ by not isolating them from the reaction mixture, but instead immediately converting them further into the compounds of the formula I-I.

On the other hand, it is possible to carry out the reaction stepwise.

The starting compounds of the formulae II-I and III are generally known. If they are not known, they can be prepared by methods known per se. Compounds of the formula II-I can be obtained, for example, by reaction of the corresponding hydroxypyrimidines built up from thiophene derivatives and CN-substituted alkylenecarboxylic acid esters with POCl₃ (Eur. J. Med. Chem. 23, 453 (1988)).

The hydroxypyrimidines are prepared either by dehydrogenation of the corresponding tetrahydrobenzothienopyrimidine compounds or by the cyclisation of 2-aminobenzothiophene-3-carboxylic acid derivatives using aldehydes or nitriles which is usual for the preparation of pyrimidine derivatives (for example Houben Weyl E9b/2).

In detail, the reaction of the compounds of the formula II-I with the compounds of the formula III is carried out in the presence or absence of an inert solvent at temperatures between about −20 and about 150°, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline, or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitriles, such as acetonitrile; sulfoxides, such as dimethyl sulfoxide (DMSO); nitro compounds, such as nitromethane or nitrobenzene; esters, such as ethyl acetate, or mixtures of the said solvents.

It is furthermore possible to convert a radical X in a compound of the formula I-I into another radical X, for example by hydrolysing an ester or a cyano group to give a COONI group.

Ester groups can be saponified, for example, using NaOH or KOH in water, water/THF or water/dioxane at temperatures between 0 and 100°. Carboxylic acids can be converted into the corresponding carbonyl chlorides, for example using thionyl chloride, and these can be converted into carboxamides. Elimination of water therefrom in a known manner gives carbonitriles.

An acid of the formula I-I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I-I can be converted into the corresponding metal salt, in particular alkali metal or alkaline earth metal salt, or into the corresponding ammonium salt using a base (for example sodium hydroxide, potassium hydroxide, sodium carbonate or potassium carbonate). Also suitable for this reaction are, in particular, organic bases which give physiologically acceptable salts, such as, for example, ethanolamine.

An acid of the formula I-I can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I-I can be converted into the corresponding metal salt, in particular alkali metal or alkaline earth metal salt, or into the corresponding ammonium salt using a base (for example sodium hydroxide, potassium hydroxide, sodium carbonate or potassium carbonate). Also suitable for this reaction are, in particular, organic bases which give physiologically acceptable salts, such as, for example, ethanolamine.

On the other hand, a base of the formula I-I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, or laurylsulfuric acid. Salts with physiologically unacceptable acids, for example picrates, can be used for the isolation and/or purification of the compounds of the formula I.

The invention furthermore relates to the pharmaceutical formulations comprising at least one phosphodiesterase V inhibitor of the formula and/or one of its physiologically acceptable salts and at least one endothelin receptor antagonist and comprising one or more excipients and/or assistants.

The pharmaceutical preparations are prepared, in particular, by non-chemical methods, in which the active ingredients are converted into a suitable dosage form together with at least one solid, liquid and/or semi-liquid excipient or assistant.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins. They can furthermore be administered as nasal sprays.

In general, the substances are preferably administered in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention therefore also relates to the use of the pharmaceutical preparations described for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

The invention relates, in particular, to the use of the formulations according to the invention for the preparation of a medicament for the treatment of high pulmonary pressure, congestive heart failure (CHF), chronic obstructive pulmonary disease (COPD), cor pulmonale and/or dextrocardiac insufficiency.

The constituents of the novel pharmaceutical [preparation are preferably administered combined. However, they can also be administered individually at the same time or successively.

The invention also relates to a set (kit) consisting of separate packs of

(a) an effective amount of [4-[4-(3-chloro-4-methoxybenzylamino)benzo-thieno(2,3-d]pyrimidin-2-yl]cyclohexanecarboxylic acid and/or physiologically acceptable salts and/or solvates thereof and

(b) an effective amount of a endothelin receptor antagonist.

The set comprises suitable containers, such as boxes, individual bottles, cartons, bags or ampoules. The set may comprise, for example, separate ampoules each containing an effective amount of [4-[4-(3-chloro-4-methoxybenzylamino)benzothieno[2,3-d]pyrimidin-2-yl]cyclohexane-carboxylic acid and/or physiologically acceptable salts and/or solvates thereof and of the endothelin receptor antagonist in dissolved or lyophilised form.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS): El (electron impact ionisation) M⁺

FAB (fast atom bombardment) (M+H)⁺

### EXAMPLE 1

Methyl 3-(4-chlorobenzothieno[2,3-d]pyrimidin-2-yl)propionate [obtainable by cyclisation of methyl 2-amino-5,6,7,8-tetrahydrobenzothiophene-3-carboxylate using methyl 3-cyanopropionate, dehydrogenation using sulfur followed by chlorination using phosphorus oxychlorideldimethylamine] and 3-chloro-4-methoxybenzylamine (“A”) in N-methylpyrrolidone are stirred at 110° for 5 hours. The solvent is removed, and the mixture is subjected to conventional work-up, giving methyl 3-[4-(3-chloro-4-methoxybenzylamino)benzotieno[2,3-d]pyrimidin-2-yl]propionate as a colourless oil.

Analogous Reaction of “A”

with methyl 2-(4-chlorobenzothieno[2,3-d]pyrimidin-2-yl)acetate gives

methyl 2-[4-(3-chloro-4-methoxybenzylamino)benzothieno[2,3-d]-pyrimidin-2-yl]acetate.

Analogous Reaction of 3,4-methylenedioxybenzylamine

with methyl 3-(4-chlorobenzothieno[2,3-d]pyrimidin-2-yl)propionate gives methyl 3-[4-(3,4-methylenedioxybenzylamino)benzothieno[2,3-d]-pyrimidin-2-yl]propionate.

Analogous Reaction of “A”

with methyl 4-(4-chlorobenzothieno[2,3-d]pyrimidin-2-yl)butyrate gives methyl 4-[4-(3-chloro-4-methoxybenzylamino)benzothieno[2,3-d]-pyrimidin-2-yl]butyrate.

Analogous Reaction of 3,4-methylenedioxybenzylamine

with methyl 4-(4-chlorobenzothieno[2,3-d]pyrimidin-2-yl)butyrate gives methyl 4-[4-(3,4-methylenedioxybenzylamino)benzothieno[2,3-d]-pyrimidin-2-yl]butyrate.

Analogous Reaction of “A”

with methyl 5-(4-chlorobenzothieno[2,3-d]pyrimidin-2-yl]valerate gives methyl 5-[4-(3-chloro-4-methoxybenzylamino)benzothieno[2,3-d]-pyrimidin-2-yl]valerate.

Analogous Reaction of 3,4-methylenedioxybenzylamine

with methyl 5-(4-chlorobenzothieno[2,3-d]pyrimidin-2-yl]valerate gives methyl 5-[4-(3,4-methylenedioxybenzylamino)benzothieno[2,3-d]-pyrimidin-2-yl]valerate.

Analogous Reaction of “A”

with methyl 7-(4-chlorobenzothieno[2,3-d]pyrimidin-2-yl)heptanoate gives methyl 7-[4-(3-chloro-4-methoxybenzylamino)benzothieno[2,3-d]-pyrimidin-2-yl]heptanoate.

Analogous Reaction of 3,4-methylenedioxybenzylamine

with methyl 7-(4-chlorobenzothieno[2,3-d]pyrimidin-2-yl)heptanoate gives methyl 7-[4-(3,4-methylenedioxybenzylamino)benzothieno[2,3-d]-pyrimidin-2-yl]heptanoate.

Analogous Reaction of “A”

with methyl 2-[4-(4-chlorobenzothieno[2,3-d]pyrimidin-2-yl)-cyclohex-1-yl]-acetate gives

methyl 2-{4-[4-(3-chloro-4-methoxybenzylamino)benzothieno[2,3d]-pyrimidin-2-yl]cyclohexyl-1-yl}acetate.

Analogous Reaction of 3,4-methylenedioxybenzylamine

with methyl 2-[4-(4-chlorobenzothieno[2,3-d]pyrimidin-2-yl)-cyclohex-1-yl]-acetate gives

methyl 2-{4-[4-(3,4-methylenedioxybenzylamino)benzothieno[2,3-d]-pyrimidin-2-yl]cyclohexyl-1-yl}acetate.

Analogous Reaction Of Benzylamine

with methyl 3-(4-chlorobenzothieno[2,3-d]pyrimidin-2-yl)propionate gives methyl 3-(4-benzylamino-benzothieno[2,3-d]pyrimidin-2-yl)propionate;

with methyl 4-(4-chlorobenzothieno[2,3-d]pyrimidin-2-yl)butyrate gives methyl 4-(4-benzylamino-benzothieno[2,3-d]pyrimidin-2-yl)butyrate;

with methyl 5-(4-chlorobenzothieno[2,3-d]pyrimidin-2-yl)valerate gives

methyl 5-(4-benzylamino-benzothieno[2,3-d]pyrimidin-2-yl)valerate.

Analogous Reaction of “A”

with methyl 4-(4-chlorobenzothieno[2,3-d]pyrimidin-2-yl)-cyclohexane-carboxylate gives

methyl 4-[4-(3-chloro-4-methoxybenzylamino)benzothieno[2,3-d]-pyrimidin-2-yl]cyclohexanecarboxylate

and reaction of 3,4-methylenedioxybenzylamine gives

methyl 4-[4-(3,4-methylenedioxybenzylamino)benzothieno[2,3-d]-pyrimid in-2-yl]cyclohexanecarboxylate.

### EXAMPLE 2

Methyl 3-[4-(3-chloro-4-methoxybenzylamino)benzothieno[2,3-d]pyrimidin-2-yl]propionate is dissolved in ethylene glycol monomethyl ether, 32% NaOH is added, and the mixture is stirred at 110 for 5 hours. After 20% HCl has been added, the mixture is extracted with dichloromethane. Addition of petroleum ether gives 3-[4-(3-chloro-4-methoxybenzylamino)-benzothieno[2,3-d]pyrimidin-2-yl]propionic acid, m.p. 2180.

The precipitated crystals are dissolved in isopropanol, and ethanolamine is added. Crystallisation gives 3-[4-(3-chloro-4-methoxybenzylamino)benzothieno[2,3-d]pyrimidin-2-yl]propionic acid, ethanolamine salt.

The following compounds are obtained analogously:

4-[4-(3-chloro-4-methoxybenzylamino)benzothieno[2,3-d]pyrimidin-2-yl]butyric acid, m.p. 2250; ethanolamine salt, m.p. 150°;

5-[4-(3-chloro-4-methoxybenzylamino)benzothie.no[2,3-d]pyrimidin-2-yl]valeric acid, m.p. 2100; ethanolamine salt, m.p. 141°;

4-(4-(3,4-methylenedioxybenzylamino)benzothieno[2,3-d]pyrimidin-2-yl]butyric acid, hydrochloride, m.p. 245°.

Analogous reaction of the esters listed under Example I gives the following carboxylic acids:

2-[4-(3-chloro-4-methoxybenzylamino)benzothieno[2,3]pyrimidin-2-yl]acetic acid,

3-[4-(3,4-methylenedioxybenzylamino)benzothieno[,3-d]pyrimidin-2-yl]propionic acid,

5-[4-(3,4-methylenedioxybenzylamino)benzothieno[2,3-d]pyrimidin-2-yl]valeric acid,

7-(4-(3-chloro-4-methoxybenzylamino)benzothieno[2,3-d]pyrimidin-2-yl]heptanoic acid,

7-[4-(3,4-methylenedioxybenzylamino)benzothieno[2,3-d]pyrimidin-2-yl]heptanoic acid,

2-{4-[4-[4-(3-chloro-4-methoxybenzylamino)benzothieno[2,3-d]pyrimidin-2-yl]cyclohexyl-1-yl}acetic acid,

2-{4-[4-(3,4-methylenedioxybenzylamino)benzothieno[2,3-d]pyrimidin-2-yl]cyclohexyl-1-yl}acetic acid,

3-(4-benzylamino-benzothieno(2,3-d]pyrimidin-2-yl)propionic acid,

4-(4-benzylamino-benzothieno[2,3-d]pyrimidin-2-yl)butyric acid,

5-(4-benzylamino-benzothieno[2,3-d]pyrimidin-2-yl)valeric acid,

4-[4-(3-chloro-4-methoxybenzylamino)benzothieno[2,3]pyrimidin-2-yl]cyclohexanecarboxylic acid, ethanolamine salt, m.p. 1670; 4-[4-(3,4-methylenedioxybenzylamino)benzothieno[2,3-d]pyrimidin-2-yl]cyclohexanecarboxylic acid, ethanolamine salt, m.p. 143°.

### EXAMPLE 3

A mixture of 1.5 g of methyl 4-(4-chlorobenzothieno[2,3-d]pyrimidin-2-yl)-phenylcarboxylate (“B”), prepared by dehydrogenation of the corresponding 5,6,7,8-tetrahydrobenzothieno[2,3-d]pyrimidine compound using sulfur followed by chlorination using phosphorus oxychloride/dimethylamine, and 1.5 g of 3-chloro-4-methoxybenzylamine in 20 ml of N-methylpyrrolidone is heated at 110° for 4 hours. After cooling, the mixture is subjected to conventional work-up, giving 2.6 g of methyl 4-[4-(3-chloro-4-methoxybenzylamino)-[1]-benzothieno[2,3-d)pyrimidin-2-yl)benzoate, m.p. 203-204°.

Analogously to Example 2, 1.2 g of the Ester Give 1.0 g of

4-[4-(3-chloro-4-methoxybenzylamino)-[1]-benzothieno[2,3-d]-pyrimidin-2-yl]benzoic acid, ethanolamine salt, m.p. 189-190°.

Analogously to Example 1, “B” and 3,4-methylenedioxybenzylamine give

methyl 4-[4-(3,4-methylenedioxybenzylamino)-[1]-benzothieno[2,3d]-pyrimidin-2-yl]benzoate, and ester hydrolysis thereof gives 4-[4-(3,4-methylenedioxybenzylamino)-1]-benzothieno[2,3-d]-pyrimidin-2-yl]benzoic acid, sodium salt, m.p. >260°.

The Following Compounds are Obtained Analogously:

4-[4-(3-chloro-4-methoxybenzylamino)-1l]-benzothieno[2,3-d]-pyrimidin-2-yl]phenylacetic acid, ethanolamine salt, m.p. 130°; and

4-[4-(3,4-methylenedioxybenzylamino)-[1]-benzothieno[2,3-d]-pyrimidin-2-yl]phenylacetic acid, ethanolamine salt, m.p. 202°.

### EXAMPLE 4

1 equivalent of 3-[4-(3-chloro-4-methoxybenzylamino)benzothieno[2,3-d]-pyrimidin-2-yl]propionic acid and 1.2 equivalents of thionyl chloride are stirred in dichloromethane for 2 hours. The solvent is removed, giving 3-[4-(3-chloro-4-methoxybenzylamino)benzothieno[2,3-d]pyrimidin-2-yl]-propionyl chloride.

The product is transferred into aqueous ammonia, and the mixture is stirred for one hour and subjected to conventional work-up, giving 3-[4-(3-chloro-4-methoxybenzylamino)benzothieno[2,3-d]pyrimidin-2-yl]propionamide.

### EXAMPLE 5

1 equivalent of DMF and 1 equivalent of oxalyl chloride are dissolved in acetonitrile at 0°. 1 equivalent of 3-[4-(3-chloro-4-methoxybenzylamino)-benzothieno[2,3-d]pyrimidin-2-yl]propionamide is then added. The mixture is stirred for a further one hour. Conventional work-up gives 3-[4-(3-chloro-4-methoxybenzylamino)benzothieno[2,3-d]pyrimidin-2-yl]propionitrile.

### EXAMPLE 6

Analogously to Examples 1, 2 and 3, reaction of the corresponding chloropyrimidine derivatives with 3,4-ethylenedioxybenzylamine gives the following carboxylic acids:

4-[4-(3,4-ethylenedioxybenzylamino)benzothieno[2,3-d]pyrimidin-2-yl]butyric acid, 3-[4-(3,4-ethylenedioxybenzylamino)benzothieno[2,3-d]pyrimidin-2-yl]propionic acid,

5-[4-(3,4-ethylenedioxybenzylamino)benzothieno[2,3-d]pyrimidin-2-yl]valeric acid,

7-[4-(3,4-ethylenedioxybenzylamino)benzothieno[2,3-d]pyrimidin-2-yl]heptanoic acid,

2-{4-[4-(3,4ethylenedioxybenzylamino)benzothieno[2,3-d]pyrimidin-2-yl]cyclohexyl-1-yl}acetic acid,

4-[4-(3,4-ethylenedioxybenzylamino)benzothieno[2,3-d]pyrimidin-2-yl]cyclohexanecarboxylic acid,

4-[4-(3,4-ethylenedioxybenzylamino)-[1]-benzothieno[2,3-d]pyrimidin-2-yl]benzoic acid, decomp. 220-230°;

4-[4-(3,4-ethylenedioxybenzylamino)-[1]-benzothieno[2,3-d]pyrimidin-2-yl]benzoic acid, ethanolamine salt, m.p. 252°;

4-[4-(3,4-ethylenedioxybenzylamino)-[1]-benzothieno[2,3-d]pyrimidin-2-yl]phenylacetic acid.

Analogous Reaction with 3,4-Dichlorobenzylamine Gives the Following Compounds:

4-[4-(3,4-dichlorobenzylamino)benzothieno[2,3-d]pyrimidin-2-yl]-butyric acid, 3-[4-(3,4-dichlorobenzylamino)benzothieno[2,3-d]pyrimidin-2-yl]-propionic acid,

5-[4-(3,4-dichlorobenzylamino)benzothieno[2,3-d]pyrimidin-2-yl]-valeric acid, ethanolamine salt, m.p. 160°;

7-[4-(3,4-dichlorobenzylamino)benzothieno[2,3-d]pyrimidin-2-yl]-heptanoic acid,

2-{4-[4-(3,4-dichlorobenzylamino)benzothieno[2,3-d]pyrimidin-2-yl]-cyclohexyl-1-yl}acetic acid,

4-[4-(3,4-dichlorobenzylamino)benzothieno[2,3-d]pyrimidin-2-yl]cyclohexanecarboxylic acid,

4-[4-(3,4dichlorobenzylamino)-[1]-benzothieno[2,3-d]pyrimidin-2-yl]-benzoic acid,

4-[4-(3,4-dichlorobenzylamino)-[1-benzothieno[2,3-d]pyrimidin-2-yl]phenylacetic acid.

Analogous Reaction with 3-chloro-4ethoxybenzylamine Gives the Following Compounds:

4-(4-(3-chloro-4-ethoxybenzylamino)benzothieno[2,3-d]pyrimidin-2-yl-]butyric acid,

3-[4-(3-chloro-4-ethoxybenzylamino)benzothieno[2,3-d]pyrimidin-2-yl]propionic acid,

5-[4-(3-chloro-4-ethoxybenzylamino)benzothieno[2,3-d]pyrimidin-2-yl]valeric acid,

7-[4-(3-chloro-4methoxybenzylamino)benzothieno[2,3-d]pyrimidin-2-yl]heptanoic acid,

2-{4-[4-(3-chloro-4-ethoxybenzylamino)benzothieno[2,3-d]pyrimidin-2-yl]cyclohexyl-1-yl}acetic acid,

4-[4-(3-chloro-4-ethoxybenzylamino)benzothieno[2,3-d]pyrimidin-2-yl]cyclohexanecarboxylic acid,

4-[4-(3-chloro-4-ethoxybenzylamino)-[1]-benzothieno[2,3-d]pyrimidin-2-yl]benzoic acid, m.p. 185-187°;

4-[4-(3-chloro-4-ethoxybenzylamino)-[1]-benzothieno[2,3-d]-pyrimidin-2-yl]phenylacetic acid.

Analogous Reaction with 3-chloro-4-isopropoxybenzylamine Gives the Following Compounds:

4-[4-(3-chloro-4-isopropoxybenzylamino)benzothieno[2,3-d]pyrimidin-2-yl]butyric acid,

3-[4-(3-chloro-4-isopropoxybenzylamino)benzothieno[2,3-d]pyrimidin-2-yl]propionic acid,

5-[4-(3-chloro-4-isopropoxybenzylamino)benzothieno[2,3d]pyrimidin-2-yl]valeric acid, ethanolamine salt, m.p. 130°;

7-[4-(3-chloro-4-isopropoxybenzylamino)benzothieno[2,3-d]pyrimidin-2-yl]heptanoic acid,

2-{4-[4-(3-chloro-4-isopropoxybenzylamino)benzothieno[2,3-d]-pyrimidin-2-yl]cyclohexyl-1-yl}acetic acid,

4-[4-(3-chloro-4-isopropoxybenzylamino)benzothieno[2,3-d]pyrimidin-2-yl]cyclohexanecarboxylic acid,

4-[4-(3-chloro-4-isopropoxybenzylamino)-[1]-benzothieno[2,3-d]-pyrimidin-2-yl]benzoic acid, m.p. 240-241°;

4-[4-(3-chloro-4-isopropoxybenzylamino)-[1]-benzothieno[2,3-d]-pyrimidin-2-yl]phenylacetic acid.

The examples below relate to pharmaceutical preparations:

### EXAMPLE A

### Injection Vials

A solution of 100 g of an active ingredient of the formula I-1,100 g of the endothelin receptor antagonist and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B

### Suppositories

A mixture of 20 g of an active ingredient of the formula I-I and 20 g of an endothelin receptor antagonist is melted with 100 g of soya lecithin and 1400 g of cocoa butter, poured into moulds and allowed to cool. Each suppository contains 20 mg of each active ingredient.

### EXAMPLE C

### Solution

A solution is prepared from 1 g of an active ingredient of the formula I-1,1 g of an endothelin receptor antagonist, 9.38 g of NaH₂PO₄.2H₂Q, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I-I and 500 mg of an endothelin receptor antagonist are mixed with 99.5 g of Vaseline under aseptic conditions.

### EXAMPLE E

### Tablets

A mixture of 1 kg of active ingredient of the formula I-I , 1 g of an endothelin receptor antagonist, 4 kg of lactose, 1.2 kg of potato starch, 0.2 kg of talc and 0.1 kg of magnesium stearate is pressed in a conventional manner to give tablets in such a way that each tablet contains 10 mg of each active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I-I and 2 kg of an endothelin receptor antagonist are introduced in a conventional manner into hard gelatine capsules in such a way that each capsule contains 20 mg of each active ingredient.

### EXAMPLE H

### Ampoules

A solution of 1 kg of active ingredient of the formula I-I and 1 kg of an endothelin receptor antagonist in 60 l of bidistilled water is sterile filtered, transferred into ampoules, lyophilised under sterile conditions and sealed under sterile conditions. Each ampoule contains 10 mg of each active ingredient.

### EXAMPLE I

### Inhalation Spray

14 g of active ingredient of the formula I-I and 14 g of an endothelin receptor antagonist are dissolved in 10 l of isotonic NaCl solution, and the solution is transferred into commercially available spray containers with pump mechanism. The solution can be sprayed into the mouth or nose.

One spray shot (about 0.1 ml) corresponds to a dose of about 0.14 mg of each active ingredient.

Compounds of the Formula I-II

The compounds of the formula I-II according to claim 1 and their salts are prepared by a process, characterised in that

a) a compound of the formula II-II

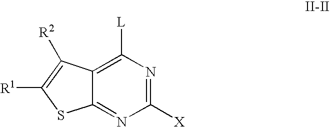

in which

R¹, R² and X are as defined above,

and L⁻ is Cl, Br, OH, SCH₃ or a reactive esterified OH group,

is reacted with a compound of the formula III

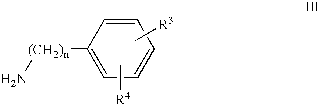

in which

R³, R⁴ and n are as defined above, or

b) a radical X in a compound of the formula I-II is converted into another radical X by, for example, hydrolysing an ester group to a COOH group or converting a COOH group into an amide or into a cyano group,

and/or in that a compound of the formula 1-11 is converted into one of its salts.

The term solvates of the compounds of the formula I-II is taken to mean adductions of inert solvent molecules onto the compounds of the formula I-II which form owing to their mutual attractive forces. Solvates are, for example, mono- or dihydrates or alcoholates.

Above and below, the radicals R¹, R², R³, R⁴, R¹, R⁶, R⁷, X, L and n are as defined for the formulae I-II, II-II and III, unless expressly stated otherwise.

A is alkyl having 1-6 carbon atoms.

In the above formulae, alkyl is preferably unbranched and has 1, 2, 3, 4, 5 or 6 carbon atoms and is preferably methyl, ethyl or propyl, furthermore preferably isopropyl, butyl, isobutyl, sec-butyl or tert-butyl, but also n-pentyl, neopentyl, isopentyl or hexyl.

X is an R⁵ or R⁶ radical which is monosubstituted by R⁷.

R⁵ is a linear or branched alkylene radical having 1-10, preferably 1-8, carbon atoms, where the alkylene radical is preferably, for example, methylene, ethylene, propylene, isopropylene, butylene, isobutylene, sec-butylene, pentylene, 1-, 2- or 3-methylbutylene, 1,1-, 1,2- or 2,2-dimethylpropylene, 1-ethylpropylene, hexylene, 1-, 2-, 3- or 4-methylpentylene, 1,1-1,2-, 1,3-, 2,2-, 2,3- or 3,3-dimethylbutylene, 1- or 2-ethylbutylene, 1-ethyl-1-methylpropylene, 1-ethyl-2-methylpropylene, 1,1,2- or 1,2,2-tri-methylpropylene, linear or branched heptylene, octylene, nonylene or decylene.

R⁵ is furthermore, for example, but-2-enylene or hex-3-enylene.

R⁶ is cycloalkylalkylene having 6-12 carbon atoms, preferably, for example, cycclopentylmethylene, cyclohexylmethylene, cyclohexylethylene, cyclo-hexylpropylene or cyclohexylbutylene.

Of the radicals R¹ and R², one is preferably H, while the other is preferably propyl or butyl, but particularly preferably ethyl or methyl. Furthermore, R¹ and R² together are alternatively preferably propylene, butylene or pentylene.

Hal is preferably F, Cl or Br, but alternatively I.

The radicals R³ and R⁴ may be identical or different and are preferably in the 3- or 4-position of the phenyl ring. They are, for example, each, independently of one another, H, OH, alkyl, F, Cl, Br or I or together are alkylene, such as, for example, propylene, butylene or pentylene, furthermore ethyleneoxy, methylenedioxy or ethylenedioxy. They are alternatively preferably each alkoxy, such as, for example, methoxy, ethoxy or propoxy.

The radical R⁷ is preferably, for example, COOH, COOCH₃, COOC₂H₁, CONH₂, CON(CHl)₂, CONHCH₃ or CN.

Throughout the invention, all radicals which occur more than once may be identical or different, i.e. are independent of one another.

The invention relates, in particular, to pharmaceutical formulations comprising an endothelin receptor antagonist and at least one compound of the formula I-II in which at least one of the said radicals has one of the preferred meanings indicated above. Some preferred groups of compounds may be expressed by the following sub-formulae Ia to Ie, which conform to the formula I-II and in which the radicals not designated in greater detail are as defined for the formula I-II, but in which

in Ia X

is R⁵ or R⁶, each of which is substituted by COOH or COOA;

in Ib

R¹ and R² are each, independently of one another, H, A or Hal, where at least one of the radicals R¹ and R² is always H,

R³ and R⁴ together are alkylene having 3-5 carbon atoms, —O—CH₂—CH₂—, —O—CH₂—O— or —O—CH₂—CH₂—O,

X is R⁵ or R⁶, each of which is substituted by COOH or COOA;

in Ic

R¹ and R² are each, independently of one another, H, A or Hal, where at least one of the radicals R¹ or R² is always ≠H,

R³ and R⁴ are each, independently of one another, H, A, OA or Hal,

R³ and R⁴ together are alkylene having 3-5 carbon atoms, —O—CH₂—CH₂—, —O—CH₂—O— or —O—CH₂—CH₂—O—,

X is R⁵ or RB, each of which is substituted by COOH or COOA,

n is 1 or 2;

in Id

R¹ and R² are each, independently of one another, H, A or Hal, where one of the radicals

R¹ and R² is always w H,

R¹ and R² together are alternatively alkylene having 3-5 carbon atoms,

R³ and R⁴ are each, independently of one another, H, A, OA or Hal,

R³ and R⁴ together are alternatively —O—CH₂—O—,

X is R⁵ which is monosubstituted by R⁷,

R⁵ is linear or branched alkylene having 1-10 carbon atoms, or —C₆H₄—CH₂—,

R⁷ is COOH or COOA,

A is alkyl having from 1 to 6 carbon atoms,

Hal is F, Cl, Br or 1, m is 1, and

n is 1 or 2;

in Ie

R¹ and R² are each, independently of one another, H, A or Hal, where one of the radicals R¹ and R² is always≠H.

R¹ and R² together are alternatively alkylene having 3-5 carbon atoms,

R³ and R⁴ are each, independently of one another, H, A, OH, OA or Hal,

R³ and R⁴ together are alternatively —O—CH₂—O—,

X is R⁵ which is monosubstituted by R⁷

R⁵ is linear or branched alkylene having 1-10 carbon atoms, or —C₆H₄—CH₂—,

R⁷ is COOH or COOA,

A is alkyl having from 1 to 6 carbon atoms, Hal is F. Cl, Br or I,

m is 1, and

n is 1 or 2;

The invention preferably relates to a formulation comprising 5-[4-(3-chloro-4-methoxybenzylamino)-5,6,7,8-tetrahydro-[1]-benzothieno[2,3-d]-pyrimidin-2-yl]valeric acid and physiologically acceptable salts and/or solvates thereof and at least one endothelin receptor antagonist. Besides the free acid, the ethanolamine salt is preferred.

Preferred endothelin receptor antagonists are those listed above under the PDE V inhibitors of the formula I.

The phosphodiesterase V inhibitors of the formula I-II and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuftgart), to be precise under reaction conditions which are known and suitable for the said reactions. Use can also be made here of variants which are known per se, but are not mentioned here in greater detail.

In the compounds of the formulae II-II or III R¹, R², R³, R⁴, X and n have the meanings indicated, in particular the preferred meanings indicated.

If L is a reactive esterified OH group, this is preferably alkylsulfonyloxy having 1-6 carbon atoms (preferably methylsulfonyloxy) or arylsulfonyloxy having 6-10 carbon atoms (preferably phenyl- or p-tolylsulfonyloxy, furthermore also 2-naphthalenesulfonyloxy).

The compounds of the formula I-II can preferably be obtained by a process in which compounds of the formula II-II are reacted with compounds of the formula III.

If desired, the starting materials can also be formed in situ by not isolating them from the reaction mixture, but instead immediately converting them further into the compounds of the formula I.

On the other hand, it is possible to carry out the reaction stepwise.

The starting compounds of the formulae II-II and III are generally known. If they are not known, they can be prepared by methods known per se. Compounds of the formula II-II can be obtained, for example, by reaction of compounds built up from thiophene derivatives and CN-substituted alkylenecarboxylic acid esters with POCl₃ (Eur. J. Med. Chem. 23, 453 (1988)).

In detail, the reaction of the compounds of the formula II-II with the compounds of the formula III is carried out in the presence or absence of an inert solvent at temperatures between about −20 and about 150°, preferably between 20 and 100°.

The addition of an acid-binding agent, for example an alkali or alkaline earth metal hydroxide, carbonate or bicarbonate or another salt of a weak acid of the alkali or alkaline earth metals, preferably of potassium, sodium or calcium, or the addition of an organic base, such as triethylamine, dimethylamine, pyridine or quinoline, or of an excess of the amine component, may be favourable.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone or dimethylformamide (DMF); nitrites, such as acetonitrile; sulfoxides, such as dimethyl sulfoxide (DMSO); nitro compounds, such as nitromethane or nitrobenzene; esters, such as ethyl acetate, or mixtures of the said solvents.

It is furthermore possible to convert a radical X in a compound of the formula I-II into another radical X, for example by hydrolysing an ester or a cyano group to give a COOH group.

Ester groups can be saponified, for example, using NaOH or KOH in water, water/THF or water/dioxane at temperatures between 0 and 100°. Carboxylic acids can be converted into the corresponding carbonyl chlorides, for example using thionyl chloride, and these can be converted into carboxamides. Elimination of water therefrom in a known manner gives carbonitriles.

An acid of the formula I-II can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I-II can be converted into the corresponding metal salt, in particular alkali metal or alkaline earth metal salt, or into the corresponding ammonium salt using a base (for example sodium hydroxide, potassium hydroxide sodium carbonate or potassium carbonate). Also suitable for this reaction are, in particular, organic bases which give physiologically acceptable salts, such as, for example, ethanolamine.

An acid of the formula I-II can be converted into the associated acid-addition salt using a base, for example by reaction of equivalent amounts of the acid and the base in an inert solvent, such as ethanol, followed by evaporation. Suitable bases for this reaction are, in particular, those which give physiologically acceptable salts.

Thus, the acid of the formula I-II can be converted into the corresponding metal salt, in particular alkali metal or alkaline earth metal salt, or into the corresponding ammonium salt using a base (for example sodium hydroxide, potassium hydroxide, sodium carbonate or potassium carbonate).

Also suitable for this reaction are, in particular, organic bases which give physiologically acceptable salts, such as, for example, ethanolamine.

On the other hand, a base of the formula I-II can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable acids. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethyl-acetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, or laurylsulfuric acid. Salts with physiologically unacceptable acids, for example picrates, can be used for the isolation and/or purification of the compounds of the formula I.

The invention furthermore relates to the pharmaceutical formulations comprising at least one phosphodiesterase V inhibitor of the formula I-II and/or one of its physiologically acceptable salts and at least one endothelin receptor antagonist and comprising one or more excipients and/or assistants.

The pharmaceutical preparations are prepared, in particular, by non-chemical methods, in which the active ingredients are converted into a suitable dosage form together with at least one solid, liquid and/or semi-liquid excipient or assistant.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearates, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants and flavours and/or a plurality of further active ingredients, for example one or more vitamins. They can furthermore be administered as nasal sprays.

In general, the substances are preferably administered in doses of between about 1 and 500 mg, in particular between 5 and 100 mg per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

The invention therefore also relates to the use of the pharmaceutical preparations described for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

The invention relates, in particular, to the use of the formulations according to the invention for the preparation of a medicament for the treatment of high pulmonary pressure, congestive heart failure (CHF), chronic obstructive pulmonary disease (COPD), cor pulmonale and/or dextrocardiac insufficiency.

The constituents of the novel pharmaceutical [preparation are preferably administered combined. However, they can also be administered individually at the same time or successively.

The invention also relates to a set (kit) consisting of separate packs of

(a) an effective amount of 5-[4-(3-chloro-4-methoxybenzylamino)-5,6,7,8-tetrahydro-[1]-benzothieno[2,3-d]pyrimidin-2-yl]valeric acid and/or physiologically acceptable salts and/or solvates thereof and

(b) an effective amount of an endothelin receptor antagonist.

The set comprises suitable containers, such as boxes, individual bottles, cartons, bags or ampoules. The set may comprise, for example, separate ampoules each containing an effective amount of 5-[4-(3-chloro-4-methoxybenzylamino)-5,6,7,8-tetrahydro-[1]-benzothieno[2,3-d]pyrimidin-; 2-yl]valeric acid and/or physiologically acceptable salts and/or solvates thereof and of the endothelin receptor antagonist in dissolved or lyophilised form.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation.

Mass spectrometry (MS): EI (electron impact ionisation) M⁺

FAB (fast atom bombardment) (M+H)⁺

### EXAMPLE 1

1.9 g of methyl 3-(4-chloro-5,6,7,8-tetrahydro-[1]-benzothieno[2,3-d]-pyrimidin-2-yl)propionate [obtainable by cyclisation of methyl 2-amino-4,5,6,7-tetrahydrobenzothiophene-3-carboxylate using methyl 3-cyano-propionate followed by chlorination using phosphorus oxychloride/dimethylamine] and 2.3 g of 3-chloro-4-methoxybenzylamine (“A”) in 20 ml of N-methylpyrrolidone are stirred at 1100 for 5 hours. The solvent is removed, and the product is subjected to conventional work-up, giving 2.6 g of methyl 3-[4-(3-chloro-4-methoxybenzylamino)-5,6,7,8-tetrahydro-[11-benzothieno(2,3-d]pyrimidin-2-yl]propionate as a colourless oil.

Analogous Reaction of “A”

with methyl 3-(4-chloro-5,6-cyclopenteno-1-benzothieno[2,3-d]pyrimidin-2-yl)propionate gives

methyl 3-[4-(3-chloro-4-methoxybenzylamino)-5,6-cyclopenteno-[1)-benzothieno[2,3-d]pyrimidin-2-yl]propionate;

with methyl 3-(4-chloro-5,6-cyclohepteno-[1]-benzothieno[2,3-d]pyrimidin-2-yl)propionate gives

methyl 3-[4-(3-chloro-4-methoxybenzylamino)-5,6-cyclohepteno-[1]-benzothieno[2,3-d]pyrimidin-2-yl]propionate;

with methyl 3-(4-chloro-6-methylthieno[2,3-d]pyrimidin-2-yl)propionate gives

methyl 3-[4-(3-chloro-4-methoxybenzylamino)-6-methylthieno[2,3-d]-pyrimidin-2-yl]propionate;

with methyl 3-(4-chloro-5,6-dimethylthieno[2,3-d]pyrimidin-2-yl)propionate gives

methyl 3-[4-(3-chloro-4-methoxybenzylamino)-5,6-dimethylthieno-[2,3-d]pyrimidin-2-yl]propionate;

with methyl 3-(4-chloro-6-ethylthieno[2,3-d]pyrimidin-2-yl)propionate gives

methyl 3-[4-(3-chloro-4-methoxybenzylamino)-6-ethylthieno[2,3-d]-pyrimidin-2-yl]propionate;

with methyl 3-(4,6-dichlorothieno[2,3-d]pyrimidin-2-yl)propionate gives

methyl 3-[4-(3-chloro-4-methoxybenzylamino)-6-chlorothieno[2,3-d]-pyrimidin-2-yl]propionate;

with methyl 2-(4-chloro-5,6,7,8-tetrahydro-(1-benzothieno[2,3-d]pyrimidin-2-yl)-acetate gives

methyl 2-[4-(3-chloro-4-methoxybenzylamino)-5,6,7,8-tetrahydro-11]-benzothieno[2,3-d]pyrimidin-2-yl]acetate.

Analogous reaction of 3,4-methylenedioxybenzylamine

with methyl 3-(4-chloro-5,6,7,8-tetrahydro-[1]-benzothieno[2,3-d]pyrimidin-2-yl)propionate gives

methyl 3-[4-(3,4-methylenedioxybenzylamino)-5,6,7,8-tetrahydro-[1]-benzothieno[2,3-d]pyrimidin-2-yl]propionate;

with methyl 3-(4-chloro-5,6-cyclopenteno-[1]-benzothieno[2,3]pyrimidin-2-yl)propionate gives

methyl 3-[4-(3,4-methylenedioxybenzylamino)-5,6-cycjopenteno-[1]-benzothieno[2,3-d]pyrimidin-2-yl]propionate;

with methyl 3-(4-chloro-5,6-cyclohepteno-[3]-benzothieno[2,3-d]pyrimidin-2-yl)propionate gives

methyl 3-[4-(3,4-methylenedioxybenzylamino)-5,6-cyclohepteno-[1]-benzothieno[2,3-d]pyrimidin-2-yl]propionate;

with methyl 3-(4-chloro-6-methylthieno[2,3-d]pyrimidin-2-yl)propionate gives

methyl 3-[4-(3,4-methylenedioxybenzylamino)-6-methylthieno[2,3-d]-pyrimidin-2-yl]propionate;

with methyl 3-(4-chloro-5,6-dimethylthieno[2,3-d]pyrimidin-2-yl)propionate gives

methyl 3-[4-(3,4-methylenedioxybenzylamino)-5,6-dimethylthieno-[2,3-d]pyrimidin-2-yl]propionate;

with methyl 3-(4-chloro-6-ethylthieno[2,3-d]pyrimidin-2-yl)propionate gives

methyl 3-[4-(3,4-methylenedioxybenzylamino)-6-ethylthieno[2,33-pyrimidin-2-yl]propionate;

with methyl 3-(4,6-dichlorothieno[2,3-d]pyrimidin-2-yl)propionate gives

methyl 3-[4-(3,4-methylenedioxybenzylamino)-6-chlorothieno[2,3-d]pyrimidin-2-yl]propionate.

Analogous Reaction of “A”

with methyl 4-(4-chloro-5,6,7,8-tetrahydro-[1]-benzothieno[2,3-d]pyrimidin-2-yl)butyrate gives

methyl 4-[4-(3-chloro-4-methoxybenzylamino)-5,6,7,8-tetrahydro-[1]-benzbthieno[2,3-d]pyrimidin-2-yl]butyrate;

with methyl 4-(4-chloro-5,6-cyclopenteno-[1]-benzothieno[2,3-d]pyrimidin-2-yl)butyrate gives

methyl 4-[4-(3-chloro-4-methoxybenzylamino)-5,6-cyclopenteno-[1]-benzothieno[2,3-d]pyrimidin-2-yl]butyrate;

with methyl 4-(4-chloro-5,6-cyclohepteno-[1]-benzothieno[2,3-d]pyrimidin-2-yl)butyrate gives

methyl 4-[4-(3-chloro-4-methoxybenzylamino)-5,6-cyclohepteno-[1]-benzothieno[2,3-d]pyrimidin-2-yl]butyrate;

with methyl 4-(4-chloro-6-methylthieno[2,3-d]pyrimidin-2-yl)butyrate gives

methyl 4-[4-(3-chloro-4-methoxybenzylamino)-6-methylthino[2,3d]-pyrimidin-2-yl]butyrate;

with methyl 4-(4-chloro-5,6-dimethylthieno[2,3-d]pyrimidin-2-yl)butyrate gives

methyl 4-[4-(3-chloro-4-methoxybenzylamino)-5,6-dimethylthieno-[2,3-d]pyrimidin-2-yl]butyrate;

with methyl 4-(4-chloro-6-ethylthieno[2,3-d]pyrimidin-2-yl)butyrate gives

methyl 4-[4-(3-chloro-4-methoxybenzylamino)-6-ethylthieno[2,3-d]-pyrimidin-2-yl]butyrate;

with methyl 4-(4,6-chloro-6-chlorothieno[2,3-d]pyrimidin-2-yl)butyrate gives

methyl 4-[4-(3-chloro-4-methoxybenzylamino)-6-chlorothieno[2,3-d]-pyrimidin-2-yl]butyrate.

Analogous Reaction of 3,4-methylenedioxybenzylamine

with methyl 4-(4-chloro-5,6,7,8-tetrahydro-[1]-benzothieno[2,3-d]pyrimidin-2-yl)butyrate gives

methyl 4-[4-(3,4-methylenedioxybenzylamino)-5,6,7,8-tetrahydro-[1]-benzothieno[2,3-d]pyrimidin-2-yl]butyrate;

with methyl 4-(4-chloro-5,6-cyclopenteno-[1]-benzothieno[2,3-d]pyrimidin-2-yl)butyrate gives

methyl 4-[4-(3,4-methylenedioxybenzylamino)-5,6-cyclopenteno-1]-benzothieno[2,3-d]pyrimidin-2-yl]butyrate;

with methyl 4-(4-chloro-5,6-cyclohepteno-[11-benzothieno[2,3-d]pyrimidin-2-yl)butyrate gives

methyl 4-[1]-(3,4-methylenedioxybenzylamino)-5,6-cyclohepteno-[1]-behzothieno[2,3-d]pyrimidin-2-yl]butyrate;

with methyl 4-(4-chloro-6-methylthieno[2,3-d]pyrimidin-2-yl)butyrate gives

methyl 4-[4-(3,4-methylenedioxybenzylamino)-6-methylthieno[2,3-d]-pyrimidin-2-yl]butyrate;

with methyl 4-(4-chloro-5,6-dimethylthieno[2,3-d]pyrimidin-2-yl)butyrate gives

methyl 4-[4-(3,4-methylenedioxybenzylamino)-5,6-dimethylthieno-[2,3-d]pyrimidin-2-yl]butyrate;

with methyl 4-(4-chloro-6-ethylthieno[2,3-d]pyrimidin-2-yl)butyrate gives

methyl 4-[4-(3,4-methylenedioxybenzylamino)-6-ethylthieno[2,3cl-pyrimidin-2-yl]butyrate;

with methyl 4-(4,6-dichlorothieno[2,3-d]pyrimidin-2-yl)butyrate gives

methyl 4-[4-(3,4-methylenedioxybenzylamino)-6-chlorothieno[2,3-d]-pyrimidin-2-yl]butyrate.

Analogous Reaction of “A”

with methyl 5-(4-chloro-5,6,7,8-tetrahydro-[1]-benzothieno[2,3-d]pyrimidin-2-yl)valerate gives

methyl 5-[4-(3-chloro-4-methoxybenzylamino)-5,6,7,8-tetrahydro-[1]-benzothieno[2,3-d]pyrimidin-2-yl]valerate;

with methyl 5-(4-chloro-5,6-cyclopenteno-[1]-benzothieno[2,3-d]pyrimidin-2-yl)valerate gives

methyl 5-[4-(3-chloro-4-methoxybenzylamino)-5,6-cyclopenteno-[1]-benzothieno[2,3-d]pyrimidin-2-yl]valerate;

with methyl 5-(4-chloro-5,6-cyclohepteno-[1]-benzothieno[2,3-d]pyrimidin-2-yl)valerate gives

methyl 5-[4-(3-chloro-4-methoxybenzylamino)-5,6-cyclohepteno-[1]benzothieno[2,3-d]pyrimidin-2-yl]valerate;

with methyl 5-(4-chloro-6-methylthieno[2,3-d]pyrimidin-2-yl)valerate gives

methyl 5-[4-(3-chloro-4-methoxybenzylamino)-6-methylthieno[2,3-d]-pyrimidin-2-yl]valerate;

with methyl 5-(4-chloro-5,6-dimethylthieno[2,3-d]pyrimid in-2-yl)valerate gives

methyl 5-[4-(3-chloro-4-methoxybenzylamino)-5,6-dimethylthieno-12,3-d]pyrimidin-2-yl]valerate;

with methyl 5-(4-chloro-6-ethylthieno[2,3-d]pyrimidin-2-yl)valerate gives

methyl 5-[4-(3-chloro-4-methoxybenzylamino)-6-ethylthieno[2,3-d]-pyrimidin-2-yl]valerate;

with methyl 5-(4,6-dichlorothieno[2,3-d]pyrimidin-2-yl)valerate gives

methyl 5-[4-(3-chloro-4-methoxybenzylamino)-6-chlorothieno[2,3-d]-pyrimidin-2-yl]valerate.

Analogous Reaction of 3,4-methylenedioxybenzylamine

with methyl 5-(4-chloro-5,6,7,8-tetrahydro-[1]-benzothieno[2,3-d]pyrimidin-2-yl)valerate gives

methyl 5-[4-(3,4-methylenedioxybenzylamino)-5,6,7,8-tetrahydro-[1]-benzothieno[2,3-d]pyrimidin-2-yl]valerate;

with methyl 5-(4-chloro-5,6-cyclopenteno-[1]-benzothieno[2,3-d]pyrimidin-2-yl)valerate gives

methyl 5-[4-(3,4-methylenedioxybenzylamino)-5,6-cyclopenteno-[1]-benzothieno[2,3-d]pyrimidin-2-yl]valerate;

with methyl 5-(4-chloro-5,6-cyclohepteno-[1]-benzothieno[2,3-d]pyrimidin-2-yl)valerate gives

methyl 5-[4-(3,4-methylenedioxybenzylamino)-5,6-cyclohepteno-[1]-benzothieno[2,3-d]pyrimidin-2-yl]valerate;

with methyl 5-(4-chloro-6-methylthieno[2,3-d]pyrimidin-2-yl)valerate gives

methyl 5-[4-(3,4-methylenedioxybenzylamino)-6-methylthieno[2,3-d]-pyrimidin-2-yl]valerate;

with methyl 5-(4-chloro-5,6-dimethylthieno[2,3-d]pyrimidin-2-yl)valerate gives

methyl 5-[4-(3,4-methylenedioxybenzylamino)-5,6-dimethylthieno-[2,3-d]pyrimidin-2-yl]valerate;

with methyl 5-(4-chloro-6-ethylthieno[2,3-d]pyrimidin-2-yl)valerate gives

methyl 5-[4-(3,4-methylenedioxybenzylamino)-6-ethylthieno[2,3-d]-pyrimidin-2-yl]valerate;

with methyl 5-(4,6-dichlorothieno[2,3-d]pyrimidin-2-yl)valerate gives

methyl 5-[4-(3,4-methylenedioxybenzylamino)-6-chlorothieno[2,3-d]-pyrimidin-2-yl]valerate.

Analogous Reaction of “A”

with methyl 7-(4-chloro-5,6,7,8-tetrahydro-[1]-benzothieno[2,3-d]pyrimidin-2-yl)heptanoate gives

methyl 7-[4-(3-chloro-4-methoxybenzylamino)-5,6,7,8-tetrahydro-[1]-benzothieno[2,3-d]pyrimidin-2-yl]heptanoate;

with methyl 7-(4-chloro-5,6-cyclopenteno-[1]-benzothieno[2,3-d]pyrimidin-2-yl)heptanoate gives

methyl 7-[4-(3-chloro-4-methoxybenzy[amino)-5,6-cyclopenteno-[1]-benzothieno[2,3-d]pyrimidin-2-yl]heptanoate;

with methyl 7-(4-chloro-5,6-cyclohepteno-[1]-benzothieno[2,3-d]pyrimidin-2-yl)heptanoate gives

methyl 7-[4-(3-chloromethoxybenzylamino)-5,6-cyclohepteno-[1]-benzothieno[2,3-d]pyrimidin-2-yl]heptanoate;

with methyl 7-(4-chloro-6-methylthieno[2,3-d]pyrimidin-2-yl)heptanoate gives

methyl 7-[4-(3-chloro-4-methoxybenzylamino)-6-methylthieno[2,13]-pyrimidin-2-yl]heptanoate;

with methyl 7-(4-chloro-5,6-dimethylthieno[2,3-d]pyrimidin-2-yl)heptanoate gives

methyl 7-[4-(3-chloro-4-methoxybenzylamino)-5,6-dimethylthieno-[2,3-d]pyrimidin-2-yl]heptanoate;

with methyl 7-(4-chloro-6-ethylthieno[2,3-d]pyrimidin-2-yl)heptanoate gives

methyl 7-[4-(3-chloro-4-methoxybenzylamino)-6-ethylthieno[2,3-d]-pyrimidin-2-yl]heptanoate;

with methyl 7-(4-chloro-6-chlorothieno[2,3-d]pyrimidin-2-yl)heptanoate gives

methyl 7-[4-(3-chloro-4-methoxybenzylamino)-6-chlorothieno[2,3-d]-pyrimidin-2-yl]heptanoate.

Analogous Reaction of 3,4-methylenedioxybenzylamine

with methyl 7-(4-chloro-5,6,7,8-tetrahydro-!1]-benzothieno[2,3-d]pyrimidin-2-yl)heptanoate gives

methyl 7-[4-(3,4-methylenedioxybenzylamino)-5,6,7,8-tetrahydro-[1]-benzothieno[2,3-d]pyrimidin-2-yl]heptanoate;

with methyl 7-(4-chloro-5,6-cyclopenteno-[1]-benzothieno[2,3-d]pyrimidin-2-yl)heptanoate gives

methyl 7-[4-(3,4-methylenedioxybenzylamino)-5,6-cyclopenteno-[1]-benzothieno[2,3-d]pyrimidin-2-yl]heptanoate;

with methyl 7-(4-chloro-5,6-cyclohepteno-[1]-benzothieno[2,3-d]pyrimidin-2-yl)heptanoate gives

methyl 7-[4-(3,4-methylenedioxybenzylamino)-5,6-cyclohepteno-[1]-benzothieno[2,3-d]pyrimidin-2-yl]heptanoate;

with methyl 7-(4-chloro-6-methylthieno[2,3d]pyrimidin-2-yl)heptanoate gives

methyl 7-[4-(3,4-methylenedioxybenzylamino)-6-methylthieno[2,3-d]-pyrimidin-2-yl]valerate;

with methyl 7-(4-chloro-5,6-dimethylthieno[2,3-d]pyrimidin-2-yl)heptanoate gives

methyl 7-[4-(3,4-methylenedioxybenzylamino)-5,6-dimethylthieno-[2,3-d]pyrimidin-2-yl]heptanoate;

with methyl 7-(4-chloro-6-ethylthieno[2,3-d]pyrimidin-2-yl)heptanoate gives

methyl 7-[4-(3,4-methylenedioxybenzylamino)-6-ethylthieno[2,3-d]-pyrimidin-2-yl]heptanoate;

with methyl 7-(4,6-dichlorothieno[2,3-d]pyrimidin-2-yl)heptanoate gives

methyl 7-[4-(3,4-methylenedioxybenzylamino)-6-chlorothieno[2,3-d]-pyrimidin-2-yl]heptanoate.

Analogous Reaction of “A”

with methyl 2-[4-(4-chloro-5,6,7,8-tetrahydro-[1]-benzothieno[2,3-d]-pyrimidin-2-yl)-cyclohexyl-1-yl]acetate gives

methyl 2-{4-[4-(3-chloro-4-methoxybenzylamino)-5,6,7,8-tetrahydro-[1]-benzothieno[2,3-d]pyrimidin-2-yl]cyclohexyl-1-yl}acetate;

with methyl 2-,[4-(4-chloro-5-ethylthieno[2,3-d]pyrimidin-2-yl)-cyclohexyl-1-yl]acetate gives

methyl 2-{4-[4-(3-chloro-4-methoxybenzylamino)-6-ethylthieno[2,3d]-pyrimidin-2-y]cyclohexyl-1-yl}acetate;

Analogous Reaction of 3,4-methylenedioxybenzylamine

with methyl 2-[4-(4-chloro-5,6,7,8-tetrahydro-[1]-benzothieno[2,3-d]-pyrimidin-2-yl)-cyclohexyl-1-yl]acetate gives

methyl 2-{4-[4-(3,4-methylenedioxybenzylamino)-5,6,7,8-tetrahydro-[1]-benzothieno[2,3-d]pyrimidin-2-yl]cyclohexyl-1-yl}acetate.

Analogous Reaction of Benzylamine

with methyl 3-(4-chloro-5,6,7,8-tetrahydro-[1]-benzothieno[2,3-d]pyrimidin-2-yl)propionate gives

methyl 3-(4-benzylamino-5,6,7,8-tetrahydro-[1]-benzothieno[2,3-d]-pyrimidin-2-yl)propionate;

with methyl 4-(4-chloro-5,6,7,8-tetrahydro-[1]-benzothieno[2,3-d]pyrimidin-2-yl)butyrate gives

methyl 4-(4-benzylamino-5,6,7,8-tetrahydro-[1]-benzothieno[2,3-d]-pyrimidin-2-yl)butyrate;

with methyl 5-(4-chloro-5,6,7,8-tetrahydro-[1]-benzothieno[2,3-d]pyrimidin-2-yl)valerate gives

methyl 5-(4-benzylamino-5,6,7,8-tetrahydro-[1]-benzothieno[2,3-d]-pyrimidin-2-yl)valerate;

with methyl 4-(4-chloro-6-methylthieno[2,3-d]pyrimidin-2-yl)butyrate gives

methyl 4-[4-benzylamino-6-methylthieno[2,3-d]pyrimidin-2-yl]butyrate;

with methyl 5-(4-chloro-6-ethylthieno[2,3-d]pyrimidin-2-yl)valerate gives

methyl 5-[4-benzylamino-6-ethylthieno[2,3-d]pyrimidin-2-yl]valerate.

### EXAMPLE 2

2.2 g of methyl 3-[4-(3-chloro-4-methoxybenzylamino)-5,6,7,8-tetrahydro-[1]-benzothieno[2,3-d]pyrimidin-2-yl]propionate are dissolved in 20 ml of ethylene glycol monomethyl ether, 10 ml of 32% NaOH are added, and the mixture is stirred at 1100 for 5 hours. After 20% HCl has been added, the mixture is extracted with dichloromethane. Addition of petroleum ether gives 2.0 g of 3-[4-(3-chloro-4-methoxybenzylamino)-5,6,7,8-tetrahydro-[1]-benzothieno[2,3-d)pyrimidin-2-yl)propionic acid, m.p. 229°.

The precipitated crystals are dissolved in 30 ml of isopropanol, and 0.5 g of ethanolamine is added. Crystallisation gives 1.3 g of 3-[4-(3-chloro-4-methoxybenzylamino)-5,6,7,8-tetrahydro-[1 ]-benzothieno[2,3-d]pyrimidin-2-yl]propionic acid, ethanolamine salt, m.p. 135°.

Analogous Reaction of the Esters Listed Under Example 1 Gives the Following Carboxylic Acids:

3-[4-(3-chloro-4-methoxybenzylamino)-5,6-cyclopenteno-[1]-benzo-thieno[2,3-d]pyrimidin-2-yl]propionic acid;

3-[4-(3-chloro-4-methoxybenzylamino)-5,6-cyclohepteno-[1]-benzo-thieno[2,3-d]pyrimidin-2-yl]propionic acid;

3-[4-(3-chloro-4-methoxybenzylamino)-6-methylthieno[2,3d]-pyrimidin-2-yl]propionic acid;

3-[4-(3-chloro-4-methoxybenzylamino)-5,6-methylthieno[2,3-d]-pyrimidin-2-yl]propionic acid;

3-[4-(3-chloro-4-methoxybenzylamino)-6-ethylthieno[2,3-d]pyrimidin-2-yl]propionic acid;

3-[4-(3-chloro-4-methoxybenzylamino)-6-chlorothieno[2,3-d]pyrimidin-2-yl]propionic acid;

2-[4-(3-chloro-4-methoxybenzylamino)-5,6, 7,8-tetrahydro-[1]-benzo-thieno[

2,3-d]pyrimidin-2-yl]acetic acid, ethanolamine salt, m.p. 126°;

3-[4-(3,4-methylenedioxybenzylamino)-5,6,7,8-tetrahydro-[1]-benzo-thieno[

2,3-d]pyrimidin-2-yl]propionic acid;

3-[4-(3,4-methylenedioxybenzylamino)-5,6-cyclopenteno-[1]-benzo-thieno[

2,3-d]pyrimidin-2-yl]propionic acid;

3-[4-(3,4-methylenedioxybenzylamino)-5,6-cyclohepteno-[1]-benzo-thieno[

2,3-d]pyrimidin-2-yl]propionic acid;

3-[4(3,4-methylenedioxybenzylamino)-6-methylthieno[2,3-d]-pyrimidin-2-yl]propionic acid;

3-[4-(3,4-methylenedioxybenzylamino)-5,6-dimethylthieno[2,3-d]-pyrimidin-2-yl]propionic acid;

3-[4-(3,4-methylenedioxybenzylamino)-6-ethylthieno[2,3-d]pyrimidin-2-yl]propionic acid;

3-[4-(3,4-methylenedioxybenzylamino)-6-chlorothieno[2,3-d]pyrimidin-2-yl]propionic acid;

4-[4-(3-chloro-4-methoxybenzylamino)-5,6,7,8-tetrahydro-[1]-benzo-thieno[

2,3-d]pyrimidin-2-yl]butyric acid;

4-[4-(3-chloro-4-methoxybenzylamino)-5,6-cyclopenteno-[1]-benzo-thieno[2,3d]pyrimidin-2-yl]butyric acid;

4-[4-(3-chloro-4-methoxybenzylamino)-5,6-cyclohepteno-tl]-benzo-thieno[2,3-d]pyrimidin-2-yl]butyric acid;

4-[4-(3-chloro-4-methoxybenzylamino)-6-methylthieno[2,3-d]-pyrimidin-2-yl]butyric acid, ethanolamine salt, m.p. 142°;

4-[4-(3-chloro-4-methoxybenzylamino)-5,6-methylthieno[2,3-d]-pyrimidin-2-yl]butyric acid;

4-[4-(3-chloro-4-methoxybenzylamino)-6-ethylthieno[2,3-d]pyrimidin-2-yl]butyric acid, ethanolamine salt, m.p. 170°;

4-[4-(3-chloro-4-methoxybenzylamino)-6-chlorothieno[2,3-d]pyrimidin-2-yl]butyric acid;

4-[4-(3,4-methylenedioxybenzylamino)-5,6,7,8-tetrahydro-[1]-benzo-thieno[2,3-d]pyrimidin-2-yl]butyric acid, ethanolamine salt, m.p. 114°;

4-[4-(3,4-methylenedioxybenzylamino)-5,6-cyclopenteno-[1]-benzo-thieno[2,3-d]pyrimidin-2-yl]butyric acid;

4-[4-(3,4-methylenedioxybenzylamino)-5,6-cyclohepteno-[1]-benzo-thieno[2,3-d]pyrimidin-2-yl]butyric acid;

4-[4-(3,4-methylenedioxybenzylamino)-6-methylthieno[2,3-d]-pyrimidin-2-yl]butyric acid, ethanolamine salt, m.p. 170°;

4-[4-(3,4-methylenedioxybenzylamino)-5,6-dimethylthieno[2,3-d]-pyrimidin-2-yl]butyric acid;

4-[4-(3,4-methylenedioxybenzylamino)-6-ethylthieno[2,3-d]pyrimidin-2-yl]butyric acid;

4-[4-(3,4-methylenedioxybenzylamino)-6-chlorothieno[2,3-d]pyrimidin-2-yl]butyric acid;

5-[4-(3-chloro-4-methoxybenzylamino)-5,6,7,8-tetrahydro-[1]-benzo-thieno[2,3-d]pyrimidin-2-yl]valeric acid, m.p. 165°; ethanolamine salt, m.p. 112°;

5-[4-(3-chloro-4-methoxybenzylamino)-5,6-cyclopenteno-[1]-benzo-thieno[2,3-d]pyrimidin-2-yl]valeric acid;

5-[4-(3-chloro-4-methoxybenzylamino)-5,6-cyclohepteno-1)]-benzo-thieno[2,3-d]pyrimidin-2-yl]valeric acid;

5-[4-(3-chloro-4-methoxybenzylamino)-6-methylthieno[₂, 3-d]-pyrimidin-2-yl]valeric acid, ethanolamine salt, m.p. 156°;

5-[4-(3-chloro-4-methoxybenzylamino)-5,6-dimethylthieno[2,3-d]-pyrimidin-2-yl]valeric acid;

5-[4-(3-chloro-4-methoxybenzylamino)-6-ethylthieno[2,3-d]pyrimidin-2-yl]valeric acid, ethanolamine salt, m.p. 156°;

5-[4-(3-chloro-4-methoxybenzylamino)-6-chlorothieno[2,3-d]pyrimidin-2-yl]valeric acid;

5-[4-(3,4-methylenedioxybenzylamino)-5,6,7,8-tetrahydro-[1]-benzo-thieno[2,3-d]pyrimidin-2-yl]valeric acid;

5-[4-(3,4-methylenedioxybenzylamino)-5,6-cyclopenteno-[1]-benzo-thieno[2,3-d]pyrimidin-2-yl]valeric acid;

5-[4-(3,4-methylenedioxybenzylamino)-5,6-cyclohepteno-[1]-benzo-thieno[2,3-d]pyrimidin-2-yl]valeric acid;

5-[4-(3,4-methylenedioxybenzylamino)-6-methylthieno[2,3-d]-pyrimidin-2-yl]valeric acid, ethanolamine salt, m.p. 167°;

5-[4-(3,4-methylenedioxybenzylamino)-5,6-dimethylthieno[2,3-d]-pyrimidin-2-yl]valeric acid;

5-[4-(3,4-methylenedioxybenzylamino)-6-ethylthieno[2,3-d]pyrimidin-2-yl]valeric acid;

5-[4-(3,4-methylenedioxybenzylamino)-6-chlorothieno[2,3-d]pyrimidin-2-yl]valeric acid;

7-[4-(3-chloro-4-methoxybenzylamino)-5,6,7,8-tetrahydro-[1]-benzo-thieno[2,3-d]pyrimidin-2-yl]heptanoic acid, ethanolamine salt, m.p. 130°;

7-[4-(3-chloro-4-methoxybenzylamino)-5,6-cyclopenteno-[1]-benzo-thieno[2,3-d]pyrimidin-2-yl]heptanoic acid;

7-[4-(3-chloro-4-methoxybenzylamino)-5,6-cyclohepteno-[11-benzo-thieno[2,3-d]pyrimidin-2-yl]heptanoic acid;

7-[4-(3-chloro-4-methoxybenzylamino)-6-methylthieno[2,3-d]pyrimidin-2-yl]heptanoic acid;

7-[4-(3-chloro-4-methoxybenzylamino)-5 ,6dimethylthieno[,3-d]pyrimidin-2-yl]heptanoic acid;

7-[4-(3-chloro-4-methoxybenzylamino)-6-ethythieno[2,3-d]pyrimidin-2-yl]heptanoic acid;

7-[4-(3-chloro-4-methoxybenzylamino)-6-chlorothieno[2,3-d]pyrimidin-2-yl]heptanoic acid;

7-[4-(3,4-methylenedioxybenzylamino)-5,6,7,8-tetrahydro-[1]-benzo-thieno[2,3d]pyrimidin-2-yl]heptanoic acid, ethanolamine salt, m.p. 137°;

7-[4-(3,4-methylenedioxybenzylamino)-5,6-cyclopenteno-[1]-benzo-thieno[2,3-d]pyrimidin-2-yl]heptanoic acid;

7-[4-(3,4-methylenedioxybenzylamino)-5,6-cyclohepteno-[1]-benzo-thieno[2,3-d]pyrimidin-2-yl]heptanoic acid;

7-[4-(3,4-methylenedioxybenzylamino)-6-methylthieno[2,3-d]-pyrimidin-2-yl]valeric acid;

7-[4-(3,4-methylenedioxybenzylamino)-5,6-dimethylthieno[2,3-d]-pyrimidin-2-yl]heptanoic acid;

7-[4-(3,4-methylenedioxybenzylamino)-6-ethylthieno[2,3-d]pyrimidin-2-yl]heptanoic acid;

7-[4-(3,4-methylenedioxybenzylamino)-6-chlorothieno[2,3-d]pyrimidin-2-yl]heptanoic acid;

2-{4-[4-(3-chloro-4-methoxybenzylamino)-5,6,7,8-tetrahydro[1]-benzothieno[2,3-d]pyrimidin-2-yl]cyclohexyl)acetic acid;

2-{4-[4-(3-chloro-4-methoxybenzylamino)-6-ethylthieno[2,3-d]-pyrimidin-2-yl]cyclohexyl}acetic acid;

2-(4-[4-(3,4-methylenedioxybenzylamino)-5,6,7,8-tetrahydro-[1]-benzothieno[2,3-d]pyrimidin-2-ylI]cyclohexyl}acetic acid; 3-(4-benzylamino-5,6,7,8-tetrahydro-[1]-benzothieno[2,3-d]pyrimidin-2-y))propionic acid, ethanoiamine salt, m.p. 126°;

4-(4-benzylamino-5,6,7,8-tetrahydro-[1]-benzothieno[2,3-d]pyrimidin-2-yl)butyric acid, ethanolamine salt, m.p. 133°;

5-(4-benzylamino-5,6,7,8-tetrahydro-[l 1-benzothieno[2,3d]pyrimidin-2-yl)valeric acid, ethanolamine salt, m.p. 135°;

4-[4-benzylamino-6-methylthieno[2,3-d]pyrimidin-2-yl]butyric acid, ethanolamine salt, m.p. 165°;

5-[4-benzylamino-6-ethylthieno[2,3-d]pyrimidin-2-yl]valeric acid, ethanolamine salt, m.p. 162°.

### EXAMPLE 3

1 equivalent of 3-[4-(3-chloro-4-methoxybenzylamino)-5,6,7,8-tetrahydro-[1]-benzothieno[2,3-d]pyrimidin-2-yl]propionic acid and 1.2 equivalents of thionyl chloride are stirred in dichloromethane for 2 hours. The solvent is removed, giving 3-[4-(3-chloro-4-methoxybenzylamino)-5,6,7,8-tetrahydro-[1]-benzothieno[2,3-d]pyrimidin-2-yl]propionyl chloride.

The product is transferred into aqueous ammonia, and the mixture is stirred for one hour and subjected to conventional work-up, giving 3-[4-(3-chloro-4-methoxybenzylamino)-5, 6,7,8-tetrahydro-[1]-benzothieno[2,3-d]-pyrimidin-2-yl]propionamide.

### EXAMPLE 4

1 equivalent of DMF and 1 equivalent of oxalyl chloride are dissolved in acetonitrile at 0°. 1 equivalent of 3-[4-(3-chloro-4-methoxybenzylamino)-5,6,7,8-tetrahydro-[1]-benzothieno[2,3-d]pyrimidin-2-yl]propionamide is then added. The mixture is stirred for a further one hour. Conventional work-up gives 3-[4-(3-chloro-4-methoxybenzylamino)-5,6,7,8-tetrahydro-[1]-benzothieno[2,3-d]pyrimidin-2-yl]propionitrile.

### EXAMPLE 5

The following compounds are obtained analogously to Examples 1 and 2:

6-[4-(3-chloro-4-mothoxybenzy!amino)-5,6,7,8-tetrahydro-(1]-benzothieno-[2,3-d]pyrimidin-2-yl]hexanoic acid, m.p. 165°;

2-[4-(3-chloro-4-methoxybenzylamino)-5,6,7,8-tetrahydro-[1]-benzothieno-[2,3-d]pyrimidin-2-yl]propionic acid, ethanolamine salt, m.p. 150°;

4-[4-(3-chloro-4-methoxybenzylamino)-5,6,7,8-tetrahydro-[1]-benzothieno[2,3-d]pyrimidin-2-yl]2,2-dimethylbutyric acid, ethanolamine salt, m.p. 130°;

4-[4-(3,4-methylenedioxybenzylamino)-5,6,7,8-tetrahydro-[1]-benzothieno-[2,3-d]pyrimidin-2-yl]2,2dimethylbutyric acid, ethanolamine salt, m.p. 126°;

5-[4-(3-chloro-4-hydroxybenzylamino)-5,6,7,8-tetrahydro-[1]-benzothieno-[2,3-d]pyrimidin-2-yl]valeric acid, m.p. 179°;

5-[4-(3,4-dichlorobenzylamino)-5,6,7,8-tetrahydro-[1]-benzothieno[2,3-d]-pyrimidin-2-yl]valeric acid, ethanolamine salt ,m.p. 1360;

5-[4-(3-chloro-4-isopropyloxybenzylamino)-5,6,7,8-tetrahydro-[1]-benzo-thieno[2,3-d]pyrimidin-2-y)]valeric acid, ethanolamine salt, m.p. 118°;

2-[4-(4-(3-chloro-4-methoxybenzylamino)-5,6,7,8-tetrahydro-[1]-benzo-thieno[2,3-d]pyrimidin-2-yl)-phenyl]acetic acid, ethanolamine salt, m.p. 119°;

2-[4-(4-(3,4-methylenedioxybenzylamino)-5,6,7,8-tetrahydro-[1]-benzo-thieno[2,3-d]pyrimidin-2-yl)-phenyl]acetic acid, m.p. 214°.

The examples below relate to pharmaceutical preparations:

### EXAMPLE A

### Injection Vials

A solution of 100 g of an active ingredient of the formula I-II , 100 g of the endothelin receptor antagonist and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of each active ingredient.

### EXAMPLE B

### Suppositories

A mixture of 20 g of an active ingredient of the formula I-II and 20 g of an endothelin receptor antagonist is melted with 100 g of soya lecithin and 1400 g of cocoa butter, poured into moulds and allowed to cool. Each suppository contains 20 mg of each active ingredient.

### EXAMPLE C

### Solution

A solution is prepared from 1 g of an active ingredient of the formula 1-11, 1 g of an endothelin receptor antagonist, 9.38 g of NaH₂PO₄.2 H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I-II and 500 mg of an endothelin receptor antagonist are mixed with 99.5 g of Vaseline under aseptic conditions.

### EXAMPLE E

### Tablets

A mixture of 1 kg of active ingredient of the formula I-III, 1 g of an endothelin receptor antagonist, 4 kg of lactose, 1.2 kg of potato starch, 0.2 kg of talc and 0.1 kg of magnesium stearate is pressed in a conventional manner to give tablets in such a way that each tablet contains 10 mg of each active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I-II and 2 kg of an endothelin receptor antagonist are introduced in a conventional manner into hard gelatine capsules in such a way that each capsule contains 20 mg of each active ingredient.

### EXAMPLE H

### Ampoules

A solution of 1 kg of active ingredient of the formula I-II and 1 kg of an endothelin receptor antagonist in 60 l of bidistilled water is sterile filtered, transferred into ampoules, lyophillsed under sterile conditions and sealed under sterile conditions. Each ampoule contains 10 mg of each active ingredient.

### EXAMPLE I

### Inhalation Spray

14 g of active ingredient of the formula I-II and 14 g of an endothelin receptor antagonist are dissolved in 10 l of isotonic NaCl solution, and the solution is transferred into commercially available spray containers with pump mechanism. The solution can be sprayed into the mouth or nose. One spray shot (about 0.1 ml) corresponds to a dose of about 0.14 mg of each active ingredient.

1. Pharmaceutical formulation comprising at least one compound of the formula I 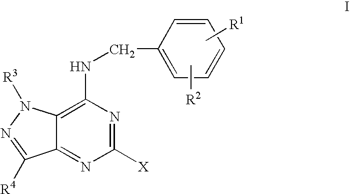
in which
R¹ and R² are each, independently of one another, H, A, OH, OA or Hal,
R¹ and R² together are alternatively alkylene having 3-5 carbon atoms, —O—CH₂—CH₂—, —CH₂—O—CH₂—, —O—CH₂—O— or —O—CH₂—CH₂—O—,
R³ and R⁴ are each, independently of one another, H or A,
X is R⁵, R⁶ or R⁷, each of which is monosubstituted by R⁸,
R⁵ is linear or branched alkylene having 1-10 carbon atoms, in which one or two CH₂ groups may be replaced by —CH═CH— groups, O, S or SO,
R⁶ is cycloalkyl or cycloalkylalkylene having 5-12 carbon atoms,
R⁷ is phenyl or phenylmethyl,
R⁸ is COOH, COOA, CONH₂, CONHA, CON(A)₂ or CN,
A is alkyl having from 1 to 6 carbon atoms, and
Hal is F, Cl, Br or I,
and/or physiologically acceptable salts and/or solvates thereof and at least one endothelin receptor antagonist.

2. Pharmaceutical formulation according to claim 1, comprising at least one compound of the formula I according to claim 1 in which
X is R⁵, phenyl or phenylmethyl, each of which is substituted by COOH, COOA, CONH₂, CONA₂, CONHA or CN;
and/or physiologically acceptable salts and/or solvates thereof and at least one endothelin receptor antagonist.

3. Pharmaceutical formulation according to claim 1, comprising at least one compound of the formula I according to claim 1 in which
R¹ and R² together are alkylene having 3-5 carbon atoms, —O—CH₂—CH₂—, —O—CH₂—O— or —O—CH₂—CH₂—O—,
X is R⁵, phenyl or phenylmethyl, each of which is substituted by COOH, COOA, CONH₂, CONA₂, CONHA or CN;
and/or physiologically acceptable salts and/or solvates thereof and at least one endothelin receptor antagonist.

4. Pharmaceutical formulation according to claim 1, comprising at least one compound of the formula I according to claim 1 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is R⁶, phenyl or phenylmethyl, each of which is substituted by COOH, COOA, CONH₂, CONA₂, CONHA or CN;
and/or physiologically acceptable salts and/or solvates thereof and at least one endothelin receptor antagonist.

5. Pharmaceutical formulation according to claim 1, comprising at least one compound of the formula I according to claim i in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R¹,
R³ is alkyl having 1-6 carbon atoms,
R⁴ is alkyl having 1-6 carbon atoms,
R⁸ is COOH or COOA,
A is alkyl having from 1 to 6 carbon atoms,
Hal is F, Cl, Br or l;
and/or physiologically acceptable salts and/or solvates thereof and at least one endothelin receptor antagonist.

6. Pharmaceutical formulation according to claim 1, comprising at least one compound of the formula I according to claim 1 in which
R¹ and R² are each, independently of one another, H, A, OH, OA or Hal,
R¹ and R² together are alternatively alkylene having 3-5 carbon atoms, —O—CH₂—CH₂—, —O—CH₂—O— or —O—CH₂—CH₂—O—,
R³ is alkyl having 1-6 carbon atoms,
R⁴ is alkyl having 1-6 carbon atoms,
X is —(CH₂)₂, R⁸, in which one CH₂ group may be replaced by O, or is 4-W-cyclohexyl, 4-R⁸-phenyl or 4-(R⁸-methyl)phenyl,
R⁸ is COOH or COOA;
and/or physiologically acceptable salts and/or solvates thereof and at least one endothelin receptor antagonist.

7. Pharmaceutical formulation according to claim 1, comprising at least one compound of the formula I according to claim 1 selected from the group consisting of
(a) 5-[7-(3-chloro-4-methoxybenzylamino)-1-methyl-3-propyl-1H-pyrazolo[4,3d]pyrimidin-5-yl]pentanoic acid;
(b) 4-[7-(3-chloro-4-methoxybenzylamino)-1-methyl-3-propyl-1H-pyrazolo[4,3-d]pyrimidin-5-y)]benzoic acid;
(c) 4-[7-(3,4-methylenedioxybenzylamino)-1-methyl-3-propyl-1H-pyrazolo[4,3-d]pyrimidin-5-yl]butyric acid;
(d) 5-(7-(benzylamino)-1-methyl-3-propyl-1H-pyrazolo[4,3-d]-pyrimidin-5-yl]pentanoic acid;
(e) [7-(3-chloro-4-methoxybenzylamino)-1-methyl-3-propyl-1H-pyrazolo[4,3-d]pyrimidin-5-ylmethoxy]acetic acid;
and/or physiologically acceptable salts and/or solvates thereof and at least one endothelin receptor antagonist.

8. Pharmaceutical formulation according to claim 1, comprising at least [7-(3-chloro-4-methoxybenzylamino)-1-methyl-3-propyl-1H-pyrazolo-[4,3-d]pyrimidin-5-ylmethoxy]acetic acid and/or physiologically acceptable salts and/or solvates thereof and at least one endothelin receptor antagonist.

9. Pharmaceutical formulation according to claims 1 to 8, in which the endothelin receptor antagonist is selected from the group consisting of bosentan, tezosentan and sitaxentan.

10. Pharmaceutical formulation according to claims 1 to 8, in which the endothelin receptor antagonist is selected from the group consisting of
a) BMS-193884 (EP 558258),
b) BMS-207940 (Pharmaprojects (13.06.97)),
c) BQ-123 (Exp.Opin.Invest.Drugs, 1997, 6, No.5, 475487),
d) SB-209670 (Exp.Opin.Invest.Drugs, 1997, 6, No.5, 475487),
e) SB-217242 (Exp.Opin.Invest.Drugs, 1997, 6, No.5, 475487),
f) SB-209598 (Trends in Pharmacol. Sci., 17, 177-81,1996),
g) TAK(-044 (Exp.Opin.Invest.Drugs, 1997, 6, No.5, 475A487),
h) Bosentan (Trends in Pharmacol. Sci., 18, 408-12, 1997),
i) PD-156707 (J.Med.Chem., 40, No.7, 1063-74,1997),
j) L-749329 (Bioorg.Med.Chem.Lett., 7, No.3, 275-280,1997),
k) L-754142 (Exp.Opin.Invest.Drugs, 1997, 6, No.5, 475-487),
l) ABT627 (J.Med.Chem., 40, No.20, 3217-27,1997),
m) A-127772 (J.Med.Chem., 39, No.5, 1039-1048, 1996),
n) A-206377 (213^{th} American Chemical Society National Meeting, San Francisco, Calif., USA, 13-17 April 1997, Poster, MEDI 193),
o) A-182086 (J.Med.Chem., 40, No.20, 3217-27, 1997),
p) EMD-93246 (211^{th} American Chemical Society National Meeting, New Orleans, USA, 1996, Poster, MEDI 143),
q) EMD-122801 (Bioorg.Med.Chem.Lett., 8, No.1, 17-22,1998),
r) ZD-1611 (Trends in Pharmacol. Sci., 18, 408-12, 1997),
s) AC-610612 (R&D Focus Drug News (18.05.98)),
t) T-0201 (70^{th} Annual Meeting of the Japanese Pharmacological Society, Chiba, Japan, 22-15 March 1997, Lecture, 0-133),
u) J-104132 (R&D Focus Drug News (15.12.97)),

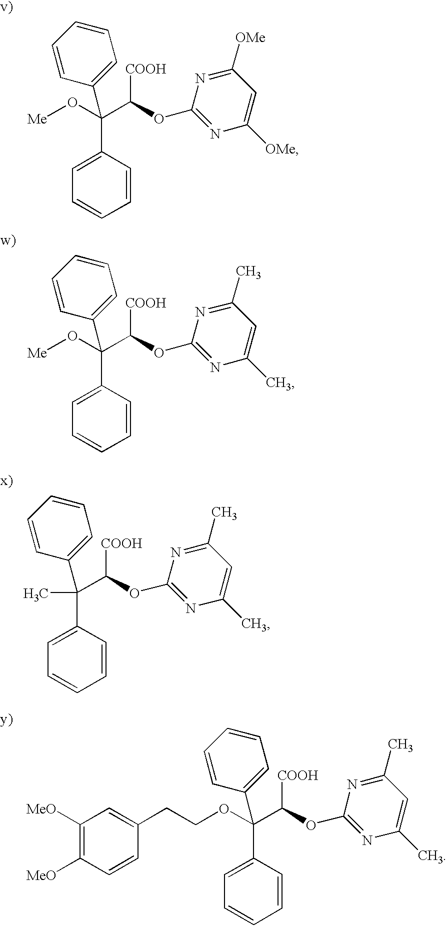

11. Pharmaceutical formulation according to claims 1 to 8, in which the endothelin receptor antagonist is selected from
a) the compounds of the formula (described in EP 0733626 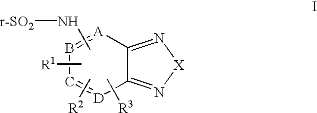
in which
-A=B-C=D- is a —CH═CH—CH═CH— group in which 1 or 2 CH has (have) been replaced by N,
Ar is Ph or naphthyl, each of which is unsubstituted or monosubstituted, disubstituted or trisubstituted by H, Hal, A, alkenyl having up to 6 carbon atoms, Ph, OPh, NO₂, NR⁴R⁵, NHCORW, CF₃, OCF₃, CN, OR⁴, COOR⁴, (CH₂)ₙCOOR⁴, (CH₂),NR⁴R⁵, —N═C═O or NHCONR⁴R⁵,
R¹, R²
and R³ are each, independently of one another, absent, H, Hal, A, CF₃, NO₂, NR⁴R⁵, CN, COOR⁴, NHCOR⁴,
R⁴ and R⁵ are each, independently of one another, H or A, or together are alternatively —CH₂—(CH₂), —CH₂—,
A is alkyl having from 1 to 6 carbon atoms,
Ph is phenyl,
X is or S,
Hal is F, Cl, Br or 1,
n is 1, 2 or 3,
and their salts, with the exception of
4-methyl-N-(2,1,3-benzothiadiazol-4-yl)benzenesulfonamide,
4-methyl-N-(2,1,3-benzothiadiazol-5-yl)benzenesulfonamide, 4-nitro-N-(2,1,3-benzothiadiazol-4-yl)benzenesulfonamide, 4-nitro-N-(2, 1,3-benzothiadiazol-5-yl)benzenesulfonamide, 4-amino-N-(2,1,3-benzo-thiadiazol-4-yl)benzenesulfonamide and 4-amino-N-(2,1,3-benzothiadiazol-5-yl)benzenesulfonamide;
b) the compounds of the formula I described in EP 0733626 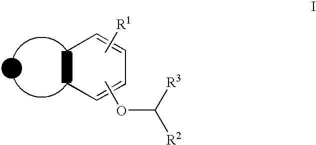
in which
X is a saturated, partially unsaturated or completely unsaturated 3- to 4-membered alkylene chain, in which from 1 to 3 carbon atoms may be replaced by N and/or from 1 to 2 carbon atoms may be replaced by 1-2 O atoms and/or 1-2 S atoms, but where at most up to 3 carbon atoms may be replaced and where, in addition, a single, double or triple substitution of the alkylene chain and/or of a nitrogen located therein by A, R′ and/or NR⁴R^{4′} may occur, and where furthermore one CH₂ group in the alkylene chain may also be replaced by a C═O group,
A is alkyl having 1-6 carbon atoms, in which one or two CH₂ groups may be replaced by O or S atoms or by —CR⁴=CR^{4′}— groups and in addition 1-7H atoms may be replaced by F,
R¹ is H or A,
R² is COOR⁴, CN, 1H-tetrazol-5-yl or CONHSO₂R⁸,
R³ is Ar,
R⁴ and R^{4′} are each, independently of one another, H, alkyl having from 1 to 6 carbon atoms or benzyl,
Ar is phenyl or naphthyl, each of which is unsubstituted or monosubstituted, disubstituted or trisubstituted by R⁵, R⁶ or R⁷, or is a 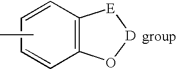
which is unsubstituted or monosubstituted or disubstituted in the phenyl part by R¹ or R⁶,
R⁵, R⁶
and R⁷ are each, independently of one another, R⁴, OR⁴, Hal, CF₃, OCF₃, OCHF₂, OCH₂F, NO₂, NR⁴R⁴, NHCOR⁴,
CN,NHSO₂R⁴, COOR⁴, COR⁴, CONHSO₂R⁸, O(CH₂)ₙR², OPh, O(CH₂)ₙOR⁴ or S(O)ₘR⁴,
R⁸ is phenyl or naphthyl, each of which is unsubstituted or monosubstituted, disubstituted or trisubstituted by A, OR¹, NR⁴R^{4′} or Hal,
E is CH₂, or 0,
D is carbonyl or [C(R⁴R⁴)]1,
Hal is F, Cl, Br or I,
m is 0, 1 or 2,
n is 0 or 2,
and their salts;
c) the compounds of the formula I described in EP 0755934 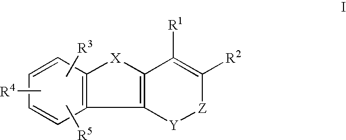
in which
—Y-Z- is —NRW—CO—, —N═C(OR⁷)— or —N═CR⁸—,
R¹ is Ar,
R² is COOR⁶, CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R⁶′, NHCOR⁶, NHSO₂R⁶, OCOR⁶, COOR⁶ or CN,
R⁶ and R^{6′} are each, independently of one another, H, alkyl having from 1 to 6 carbon atoms, benzyl or phenyl,
R⁷ is (CH₂)ₙAr,
R³ is Ar or OAr,
Ar is phenyl which is unsubstituted or monosubstituted, disubstituted or trisubstituted by R⁹, R¹⁰ or W¹¹, or is unsubstituted naphthyl or a 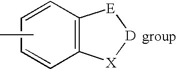
which is unsubstituted or monosubstituted or disubstituted in the phenyl part by R⁹ or R¹⁰, or is a 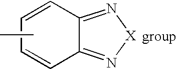
which is unsubstituted or monosubstituted or disubstituted in the cyclohexadienyl part by R⁹ or R¹⁰,
R⁹, R¹⁰
and R¹¹ are each, independently of one another, R⁶, OR⁶, Hal, CF₃, OCF₃, OCHF₂, OCH₂F, NO₂, NR⁶R⁶, NHCOR⁶, CN, NHSO₂R⁶, COOR⁶, COR⁶, CONHSO₂Ar, O(CH₂)ₙR², O(CH₂),OR⁶ or S(O),R⁶,
E is CH₂, S or O,
D is carbonyl or [C(R⁶R^{6′})],
Hal is F, Cl, Br or 1,
X is O or S,
m is 0, 1 or 2,
n is 1 or 2,
and their salts;
d) the compounds of the formula I described in EP 0757039 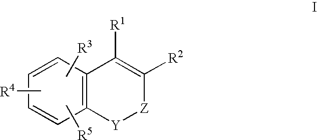
in which
—Y-Z- is —NR⁷—CO—, —N═C(OR⁷)— or —N═CR⁸—,
R¹ is Ar,
R² is COOR⁶, (CH₂)ₙCOOR⁶, CN, 1H-tetrazol-5-yl or CONHSO₂Ar,
R³, R⁴
and R⁵ are each, independently of one another, R⁸, OR⁶, S(O)ₘR⁶, Hal, NO₂, NR⁶R⁶′, NHCOR⁶, NHSO₂R⁶, OCOR⁶, COR⁶, COOR⁶ or CN, where R³ and R⁴ together may alternatively be an O(CH₂),O group,
R⁶ and R^{6′} are each, independently of one another, H, alkyl having from 1 to 6 carbon atoms, benzyl or phenyl,
R⁷ is (CHO₂)ₙAr,
R⁸ is Ar or OAr,
Ar is phenyl which is unsubstituted or monosubstituted, disubstituted or trisubstituted by R⁹, R¹⁰ or R¹¹, or is unsubstituted naphthyl or a 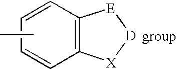
which is unsubstituted or monosubstituted or disubstituted in the phenyl part by R⁹ or R¹⁰, or is 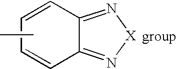
which is unsubstituted or monosubstituted or disubstituted in the cyclohexadienyl part by R⁹ or R¹⁰,
R⁹, R¹⁰
and R¹¹ are each, independently of one another, R⁶, ORW, Hal, CF₃, OCF₃, OCHF₂, OCH₂F, NO₂, NR⁶R^{6′}, NHCOR⁸, CN, NHSO₂R⁶, COOR⁶, COR⁶, CONHSO₂Ar, O(CH₂)ₙR², O(CH₂),OR⁶ or S(O)ₘR⁶,
E is CH₂,S or O,
D is carbonyl or [C(R⁶R⁶)]ₙ,
X is O or S,
Hal is F, Cl, Br or 1,
m is 0, 1 or 2,
n is 1 or 2,
and their salts;
e) the compounds of the formula I described in EP 0796250 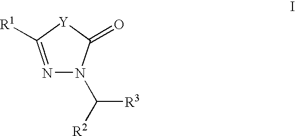
in which
Y is —C(R⁴R^{4′})—C(R⁴R^{4′})—, —CR⁴═CR⁴′— or —C(R⁴R⁴)—S—,
R¹ is Het, Ar, RW or R⁴,
R² is Ar or a 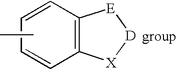
which is unsubstituted or monosubstituted or disubstituted in the phenyl part by A, R³, OR⁴, NH₂, NHA, NA₂, NO₂, CN, Hal, NHCOR⁴, NHSO₂R⁴, COOR⁴, COR⁴, CONHSO₂RW, O(CH)ₙR³, OPh, O(CH₂),OR⁴ or S(O)ₘR⁴, or a 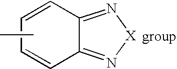
which is unsubstituted or monosubstituted or disubstituted in the cyclohexadienyl part by A, R³, OR⁴, NH₂, NHA, NA₂, NO₂, CN, Hal, NHCOR⁴, NHSO₂R⁴, COOR⁴, COR⁴, CONHSO₂RG, O(CH₂),R³, OPh, O(CH₂),OR⁴ or S(O)ₘR⁴,
R³ is CN, COOH, COOA, CONHSO₂R⁵ or I H-tetrazol-5-yl,
R⁴ and R^{4′} are each, independently of one another, H, A, or phenyl or benzyl, each of which is unsubstituted or monosubstituted by alkoxy,
R⁵ is A or Ar,
R⁶ is phenyl or naphthyl, each of which is unsubstituted or monosubstituted, disubstituted or trisubstituted by A, OR⁵, NH₂, NHA, NA₂, NO₂, CN or Hal,
A is, alkyl having 1-6 carbon atoms, in which one or two CH₂ groups may be replaced by O or S atoms or by —CR⁴═CR⁴′-groups and in addition 1-7H atoms may be replaced by F, or benzyl,
Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂R⁴, COOR⁴, COR⁴, CONHSO₂R⁶, O(CH₂),R³,
OPh, O(CHD₂)ₙOR⁴ or S(O)ₘR⁴,
Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂ and/or carbonyl oxygen,
D is carbonyl or [C(R⁴R⁴)]ₙ,
E is CH₂, S or O,
Hal is F. Cl, Br or I,
X is O or S,
m is 0, 1 or 2,
n is 1 or 2,
and their salts;
f) the compounds of the formula I described in WO 9719077 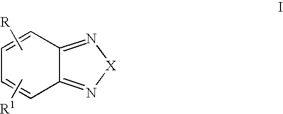
in which
R is 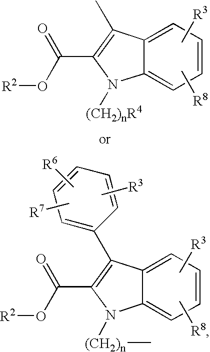
X is O or S,
R¹ is H, Hal, OH, OA, A, alkylene-O-A, NO₂, NH₂, NH-acyl, SO₂NH₂, SO₃-A, SO₂NHA, CN or formyl,
R² is H or A,
R⁹, R⁵, R⁶
R⁷ and R⁸ are each, independently of one another, H, Hal, OH, OA, O-alkylene-R⁴, A, S-A, NO₂, NH₂, NHA, NA₂, NH-acyl, NHSO₂A, NHSO₂R⁴, NASO₂A, NASO₂—R⁴, NH(CO)NH₂, NH(CO)NHA, formyl, NH(CO)NH-phenyl, NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CH₂)ₙOR², CH₂OH or CH₂OA,
R³ and R⁴ together are alternatively —O—CH₂—O—, —O—CH₂—CH₂O—, —O—CH₂—CH₂—, —O—CF₂—O— or —O—CF₂—CF₂—O—,
R⁴ is phenyl which is unsubstituted or monosubstituted or polysubstituted by R³ and/or R⁶,
A is alkyl having 1-6 carbon atoms,
Hal is fluorine, chlorine, bromine or iodine,
n is 1 or 2, and their salts;
g) the compounds of the formula I described in WO 9730982 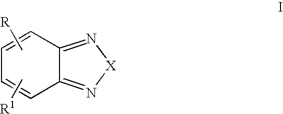
in which
R is 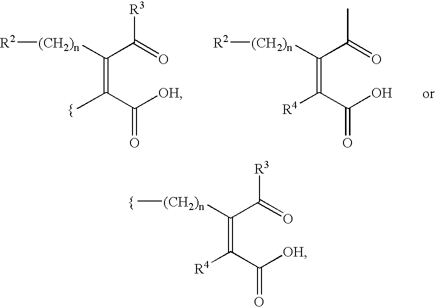
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by Hal, OH, OA, O-alkylene-Rr, A, S-A, SOA, SO₂A, SOR⁵, SO₂R⁵, NO₂, NH₂, NHA, NA₂₁ NH-acyl, NHSO₂A, NHSO₂R⁵, NAS02A, NASO₂—R⁸, NH(CO)NH₂, NH(CO)NHA, formyl, NH(CO)NHR⁶, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂),COOA, O(CH₂)ₙCOOH, O(CH₂)ₙOH, O(CH₂),OA, CH₂OH, CH₂OA, COOH, COOA, CH₂COOH or CH₂COOA,
or is a 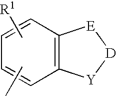
group or a 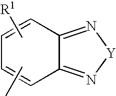
group, where
R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CHJNCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR⁶″-groups and/or 1-7H atoms may be replaced by F,
D is carbonyl or [C(R⁶R⁶)]ₘ,
E is CH₂, S or O,
Y is O or S,
R¹ and R^{6′} are each, independently of one another, H, F or A,
Hal is fluorine, chlorine, bromine or iodine,
n is 1 or 2, and
m is 1 or 2,
or a tautomeric cyclised form, and the (E)-isomers and the salts of all isomers;
h) the compounds of the formula I described in WO 9730996 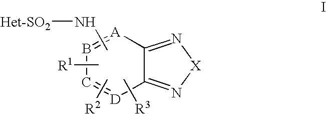
in which
-A=B-C=D- is a —CH═CH—CH═CH— group, in which, in addition, 1 or 2 CH may be replaced by N,
Het is a monocyclic or bicyclic, saturated, unsaturated or aromatic heterocyclic radical having from 1 to 4 N, O and/or S atoms which is unsubstituted or substituted by -Z-R⁶,
R¹,R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
R⁶ is a phenyl radical, benzothiadiazol-5-yl or benzoxadiazol-5-yl radical, each of which is unsubstituted or monosubstituted, disubstituted or trisubstituted by R⁷, R⁸ and/or R⁹,
R¹, R⁸
and R⁹ are each, independently of one another, A, O-A, CN, COOH, COOA, Hal, formyl, —CO-A, and R⁷ and R⁸ are alternatively —O—(CH₂)ₘ—O—,
A is alkyl having from 1 to 6 carbon atoms,
X is O or S,
z is —CO—, —CONH—, —CO—(CH₂)ₙ—, —CH═CH—, —(CH₂)ₙ—, —CONHCO—, —NHCONH—, —NHCOO—, —O—CONH—, —CO—O— or —O—CO—,
Hal is F, Cl, Br or I,
m is 1 or 2, and
n is 1, 2 or 3,
and their salts; i) the compounds of the formula I described in DE 19609597 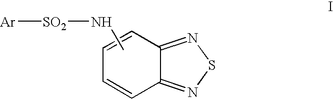
in which
Ar is naphthyl which is monosubstituted by NH₂, NHA or NA₂, and
A is alkyl having from 1 to 6 carbon atoms,
and their physiologically acceptable salts;
j) the compounds of the formula I described in DE 19612101 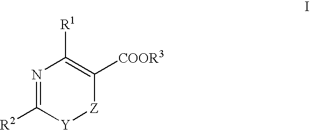
in which
—Y-Z- is —NR⁴—CO or —N═CR⁵—,
R¹ is Ar,
R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by RW, ORW or Hal,
R³ and R^{3′} are each, independently of one another, H, alkyl having 1-6 carbon atoms or benzyl,
R⁴ is CH₂Ar,
R⁵ is OCH₂Ar,
Ar is phenyl which is unsubstituted or monosubstituted, disubstituted or trisubstituted by R⁶, R⁷ or R⁸, or a 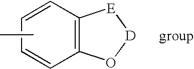
which is unsubstituted or monosubstituted in the phenyl part by R⁶, or a 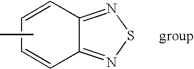
which is unsubstituted or monosubstituted in the cyclohexadienyl part by R⁸,
E is CH₂ or 0,
D is carbonyl or (CH₂)ₙ,
E and D together are alternatively CH═CR⁹,
R⁶, R′″ are each, independently of one another, R³, R³ or Hal,
R⁷ is R³, OR³, Hal, NO₂, NH₂, NHR³, NR³R³, NHCOR³, COOR³, O(CH₂)ₙR³ or O(CH₂),OR³,
R⁸ is Ph which is unsubstituted or monosubstituted, disubstituted or trisubstituted by R³, OR³, Hal, NO₂, NH₂, NHR⁶, NR⁶R^{6′}, NHCOR³ or COOR³,
R⁹ is H, OH, CH₂OH or COOR³,
Hal is F, Cl, Br or 1,
Ph is phenyl,
m is 0 or 1,
n is 1 or 2,
and their salts;
k) the compounds of the formula I described in WO 9827091 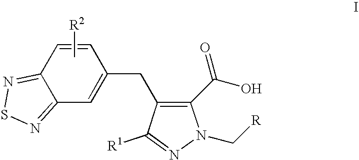
in which
R is phenyl which is unsubstituted or monosubstituted, disubstituted or trisubstituted by R³, R⁴ or R⁶, or 2,1,3-benzothiadiazolyl which is unsubstituted or mono-substituted by R²,
R¹ is A, in which 1-7H atoms may be replaced by F,is —S-A, —O-A, is phenyl or -alkylene-phenyl, each of which is unsubstituted or monosubstituted by R³, or is thienyl which is unsubstituted or monosubstituted by R³,
R² is A, F, Cl, Br or —O-A,
R³, R⁴
and R⁵ are each, independently of one another, A, —O-A, —S-A, —O-alkylene-COOH, -alkylene-COOH or COOH,
R³ and R⁴ together are alternatively —O—CH₂—O—, and
A is alkyl having 1-7 carbon atoms,
and their salts;
l) the compounds of the formula I described in WO 9827077 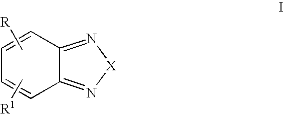
in which
R is 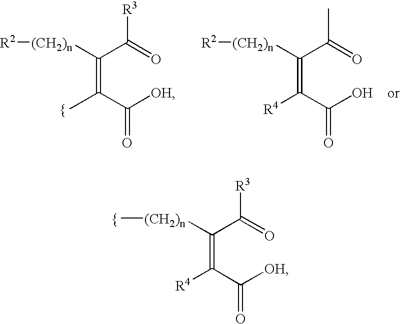
X is O or S,
R¹ is H, Hat, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or poly-substituted by R⁷, where R² is additionally A or cycloalkyl, or are 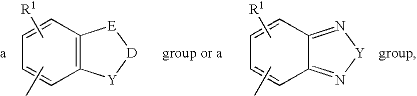
with the proviso that at least one of the radicals R², R³ or R⁴ is an R⁸ radical which is unsubstituted or monosubstituted or polysubstituted by R⁷,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)COOA, O(CHONCOOH, O(CH₂),OH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR⁶′-groups and/or 1-7H atoms may be replaced by F,
D is carbonyl or [C(R⁶R^{6′})]ₘ,
E is sCH₂, S or O,
Y is O or S,
R⁶ and R^{6′} are each, independently of one another, H, F or A,
R⁷ is Hal, OH, OA, O-alkylene-R⁵, A, S-A, S-OA, SO₂A, S—OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
R⁸ is a 5-7-membered heterocyclic radical having 1-4 N, O and/or S atoms or is 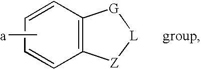
G and Z are each, independently of one another, —CH═, N, O or S,
L is —CH═, —CH═CH— or —CH₂—CH₂—CH₂—,
Hal is fluorine, chlorine, bromine or iodine,
n is 0, 1 or 2, and
m is 1 or 2,
or a tautomeric cyclised form, and the (E)-isomers and the salts of all isomers;
m) the compounds of the formula I described in WO 9841515 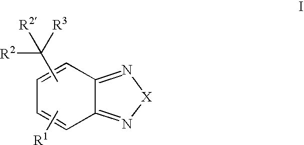
in which
X is O or S,
R¹ is H, Hal, OH, OA, A, NO₂, NH₂, NHA, NAA′, NHCOR⁴, NHCOR⁶, NHSO₂R⁴, NHSO₂R⁶, S(O)ₘR⁶, SO₃H, SO₂NR⁴R⁴ or formyl,
R² and R² are each, independently of one another, A, (CH₂)ₙAr, (CHO₂)ₙHet, CH₂COAr, CH₂COHet or OAr,
R^{2′} is additionally also H,
R³ is COOR⁴, CN, 1H-tetrazol-5-yl or CONHSO₂R⁵,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ and R^{7′} are each, independently of one another, H or alkyl having 1-6 carbon atoms,
A and A′ are each, independently of one another, alkyl having 1-6 carbon atoms, in which one or two CH₂ groups may be replaced by O or S atoms or by —CR⁷═CR⁷′-groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁴, COOR⁴, OPh, CONH₂, CONHA, CONAA′, COR⁴, CONHSO₂R⁴, CONHSO₂R⁶, O(CH₂)ₙCOOR⁴, O(CH₂)OR⁴, SO₃H, SO₂NR⁴R⁴, S(O)ₘR⁶ or S(O)ₘR⁴,
Het is a monocyclic or bicyclic, saturated, unsaturated or aromatic heterocyclic radical having 1-4 N, O and/or S atoms, bonded via N or C, which may be unsubstituted or monosubstituted, disubstituted or trisubstituted by Hal, A, R³, NH₂, NHA, NAA′, NO₂ and/or ═O,
Hal is fluorine, chlorine, bromine or iodine,
m is 0, 1 or 2, and
n is 1 or 2,
where, if R² is CH₂COAr and R¹ is H, R³ is not COOA, and salts thereof;
n) the compounds of the formula I described in WO 9841521 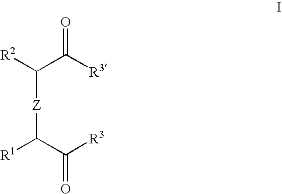
in which
Z is a single or double bond,
R¹ is a 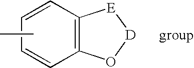
which is unsubstituted or monosubstituted in the phenyl part by R⁷, or is a 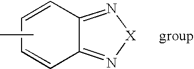
which is unsubstituted or monosubstituted in the cyclohexadienyl part by R⁷,
R² is A, Ar-(CH₂)ₘ, cycloalkyl-(CH₂)ₘ, Het-(CH₂)ₘ or R¹-(CH₂)ₘ,
R³ and R^{3′} are each, independently of one another, OR⁴, NHSO₂R⁵, NH₂, NHA or NAA′,
R³ and R^{3′} together are alternatively —O—, forming a cyclic anhydride,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ is A, COOR⁴, CN, 1H-tetrazol-5-yl, CON HSO₂R⁵, Hal, OR⁴, NO₂, NH₂, NHA, NAA′, NHCOR⁴, NHCOR⁶, NHSO₂R⁴, NHSO₂R⁶, S(O)ₖR⁴, S(O)ₖR⁴, SO₂NR⁴R^{4′} or formyl,
R⁸ and R^{5′} are each, independently of one another, H or alkyl having 1-6 carbon atoms,
E is CH₂ or O,
D is carbonyl or (CR⁴R^{4′})ₙ,
E and D together are alternatively CR⁴═R⁴,
X is S or O,
A and A′ are each, independently of one another, alkyl having 1-6 carbon atoms, in which one or two CH₂ groups may be replaced by O or S atoms or by —CR⁸═CR^{8′} groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂),COOR⁴, O(CH₂)ₙOR⁴, SO₂NR⁴R^{4′}, S(O)ₖR⁶ or S(O)ₖR⁴,
Het is a monocyclic or bicyclic, saturated, unsaturated or aromatic heterocyclic radical having 1-4 N, O and/or S atoms, bonded via N or C, which may be unsubstituted, monosubstituted or disubstituted or trisubstituted by Hal, A, COOR⁴, CN, 1H-tetrazol-5-yl, CONHSO₂R⁵, NH₂, NHA, NAA′, NO₂ and/or ═O,
Hal is fluorine, chlorine, bromine or iodine,
k is 0, 1 or 2,
m is 0, 1 or 2, and
n is 1 or 2,
and the (Z)- and (E)-isomers and the salts of all isomers;
o) the compounds of the formula I described in WO 9842702 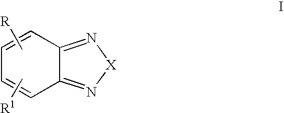
in which
R 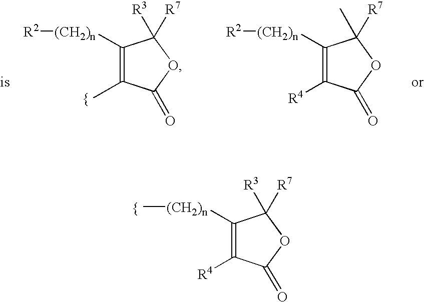
X and Y are each, independently of one another, O or S,
R¹ is H, Hal, OH, OA, A, alkylene-O-A, NO₂, NH₂, NH-acyl, SO₂NH₂₁ SO₂A, SO₂N HA, ON or formyl,
R², R³
and R⁴ are each, independently of one another, a phenyl group which is unsubstituted or monosubstituted or polysubstituted by Hal, OH, OA, O-alkylene-R¹, A, S-A, S-OA, SO₂A, S—OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂),COOA, O(CH₂),COOH, O(CH₂),OH, O(CH₂),OA, CH₂OH, CH₂OA, COOH, COOA, CH₂COOH or CH₂COOA, 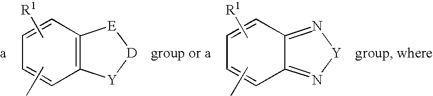
R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂),COOA, O(CH₂),COOH, O(CH₂)ₙOH, O(CH₂),OA, CH₂OH, CH₂OA, COOH, COOA, CH₂COOH or CH₂COOA,
A is alkyl having 1-6 carbon atoms, in which one or two CH, groups may be replaced by O or S atoms or by —CR⁶═CR⁶′-groups and/or 1-7H atoms may be replaced by F,
D is carbonyl or [C(R⁶R^{6′})]ₘ,
E is CH₂, S or O,
R⁶ and R^{6′} are each, independently of one another, H, F or A,
R⁷ is —O—C(═Y)-NH—R⁸,
R⁸ is alkyl having 1-10 carbon atoms which is unsubstituted or monosubstituted or disubstituted by R⁸ and in which 1-2 carbon atoms may be replaced by O and/or S, and/or may be substituted by ═O, or cycloalkyl, in which 1-2 carbon atoms may be replaced by N, O and/or S,
R⁹ is phenyl which is unsubstituted or monosubstituted or disubstituted by Hal, or is naphthyl, A-O—C(═O)— or Hal,
Hal is fluorine, chlorine, bromine or iodine,
n is 0, 1 or 2, and
m is 1 or 2,
and salts thereof;
p) the compounds of the formula I described in WO 9842709 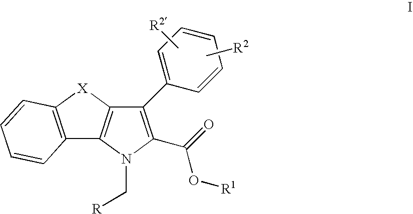
in which
X is N—R³, O or S,
R is 2,1,3-benzothiadiazol-4- or 5-yl or 2,1-benzoiso-thiazol-5- or 6-yl, each of which is unsubstituted or monosubstituted or disubstituted by R² and/or R^{2′}, or phenyl which is unsubstituted or monosubstituted, disubstituted or trisubstituted by R² and/or R^{2′},
R¹ is H or A,
R² and R^{2′} are each, independently of one another, H, A, OH, OA, Hal, OCF₃, OCHF₂, —O—CO-A, —O-alkylene-COOR¹, —O-alkylene-CH₂—OR¹, or OCH₂-phenyl or —O—CO-phenyl, each of which is unsubstituted or monosubstituted or disubstituted in the phenyl part by R⁴ and/or R⁴′,
R² and R^{2′} together are alternatively —OCH₂O—, —OCH₂CH₂O— or —OCH₂CH₂—,
R³ is H, A, alkylene-O-A, —CO-OA, or alkylene-phenyl which is unsubstituted or mono-substituted or disubstituted in the phenyl part by R⁴ and/or R^{4′},
R⁴ and R^{4′} are each, independently of one another, H, A, OH, OA, Hal, COOR¹ or CH₂OR¹,
A is alkyl having 1-6 carbon atoms,
Hal is fluorine, chlorine, bromine or iodine,
and their salts;
q) the compounds of the formula I described in WO 9905132 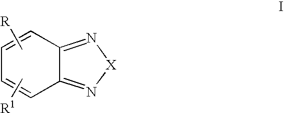
in which
R 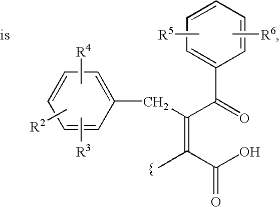
X is O or S,
R¹ is H, Hal, OA or A,
R², R³, R⁵,
and R⁶ are each, independently of one another, H, Hal, A, OA or R⁴,
R⁴ is —O—(CH₂)ₙ-Cy,
Cy is cycloalkyl having 3-8 carbon atoms;
A is alkyl having 1-6 carbon atoms, in which one or two CH₂ groups may be replaced by O or S atoms or by —CR⁵═CR^{5″} groups and/or 1-7H atoms may be replaced by F,
R⁵ and R^{5′} are each, independently of one another, H, F or A,
Hal is fluorine, chlorine, bromine or iodine,
n is 0, 1 or 2,
or a tautomeric cyclised form, and the (E)-isomers and the salts of all isomers.

12. Pharmaceutical formulation according to one of the preceding claims, comprising one or more excipients and/or assistants.

13. Use of a pharmaceutical preparation according to one of claims 1 to 12 for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

14. Use according to claim 13 for the preparation of a medicament for the treatment of high pulmonary pressure, congestive heart failure (CHF), chronic obstructive pulmonary disease (COPD), cor pulmonale and/or dextrocardiac insufficiency.

15. Set (kit) consisting of separate packs of (a) an effective amount
of [7-(3-chloro-4-methoxybenzylamino)-1-methyl-3-propyl-1H-pyrazolo[4,3-d]pyrimidin-5-ylmethoxy]acetic acid and/or physiologically acceptable salts and/or solvates thereof and
(b) an effective amount of an endothelin receptor antagonist.

16. Pharmaceutical formulation comprising at least one compound of the formula I-I 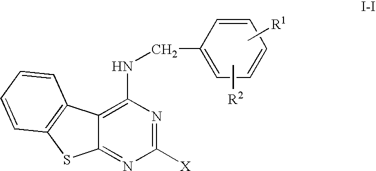
in which
R¹ and R² are each, independently of one another, H, A, OA, OH or Hal,
R¹ and R² together are alternatively alkylene having 3-5 carbon atoms, —O—CH₂—CH₂—, —CH₂—O—CH₂—, —O—CH₂—O— or —OCH₂—CH₂—O—,
X is R⁴, R⁵ or R^{5″}, each of which is monosubstituted by R⁷,
R⁴ is linear or branched alkylene having 1-10 carbon atoms, in which one or two CH₂ groups may be replaced by —CH═CH— groups,
R⁵ is cycloalkyl or cycloalkylalkylene having 5-12 carbon atoms,
R⁶ is phenyl or phenylmethyl,
R⁷ is COOH, COOA, CONH₂, CONHA, CON(A)₂ or CN,
A is alkyl having from 1 to 6 carbon atoms, and
Hal is F, Cl, Br or 1,
and/or physiologically acceptable salts and/or solvates thereof and at least one endothelin receptor antagonist.

17. Pharmaceutical formulation according to claim 16, comprising at least one compound of the formula I-II according to claim 16 in which X is R⁴, phenyl or phenylmethyl, each of which is substituted by COOH, COOA, CONH₂, CONA₂, CONHA or CN.

18. Pharmaceutical formulation according to claim 16, comprising at (east one compound of the formula I-II according to claim 16 in which R¹ and R² together are alkylene having 3-5 carbon atoms,
—O—CH₂—CH₂—, —O—CH₂—O— or —O—CH₂—CH₂—O—,
X is R⁴, phenyl or phenylmethyl, each of which is substituted by COOH, COOA, CONH₂, CONA₂, CONHA or CN.

19. Pharmaceutical formulation according to claim 16, comprising at (east one compound of the formula I-II according to claim 16 in which
R¹ and R² are each, independently of one another, H, A, OA or Hal,
R¹ and R² together are alkylene having 3-5 carbon atoms, —O—CH₂—CH₂—, —O—CH₂—O— or —O—CH₂₇CH₂—O—,
X is R⁴, phenyl or phenylmethyl, each of which is substituted by COOH, COOA, CONH₂, CONA₂, CONHA or CN.

20. Pharmaceutical formulation according to claim 16, comprising at least one compound of the formula I-II according to claim 16 in which
R¹ and R² are each, independently of one another, H, A, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R⁷
R⁷ is COOH or COOA,
A is alkyl having from 1 to 6 carbon atoms,
Hal is F, Cl, Br or I.

21. Pharmaceutical formulation according to claim 16, comprising at least one compound of the formula I-II according to claim 16 in which
R¹ and R² are each, independently of one another, H, A, OA or Hal,
R¹ and R² together are alternatively alkylene having 3-5 carbon atoms, —O—CH₂—CH₂—, —O—CH₂—O— or —O—CH₂—CH₂—O—,
X is alkylene having 2-5 carbon atoms, cyclohexyl, phenyl or phenylmethyl, each of which is monosubstituted by R⁷,
R⁷ is COOH or COOA,
A is alkyl having from 1 to 6 carbon atoms,
Hal is F. Cl, Br or I.

22. Pharmaceutical formulation according to claim 16, comprising at least one compound of the formula I-II according to claim 16, selected from the group consisting of
(a) 3[4-(3-chloro-4-methoxybenzylamino)benzo[4,5]thieno[2,3-d]-pyrimidin-2-yl]propionic acid;
(b) 4-[4-(3,4-methylenedioxybenzylamino)benzo[4,5]thienol-2,3-d)-pyrimidin-2-yl]butyric acid;
(c) 7-[4-(3,4-methylenedioxybenzylamino)benzo[4,5]thieno[2,3-d]-pyrimidin-2-yl]heptanoic acid;
(d) 7-[4-(3-chloro-4-methoxybenzylamino)benzo[4,5]thieno[2,3-d]-pyrimidin-2-yl]heptanoic acid;
(e) 5-[4-(3-chloro-4-methoxybenzylamino)benzo[4,5]thieno[2,3-d]pyrimidin-2-yl]valeric acid;
(f) 2-{4-[4-(3-chloro-4-methoxybenzylamino)benzo[4,5]thieno-[2,3-d]pyrimidin-2-yl]cyclohexyl-1-yl}acetic acid;
(g) 4-[4-(3,4-methylenedioxybenzylamino)benzo[4,5]thieno[2,3-d]-pyrimidin-2-yl]cyclohexanecarboxylic acid;
(h) 4-[4-(3,4-methylenedioxybenzylamino)benzo[4,5]thieno[2,3-d]-pyrimidin-2-yl]benzoic acid;
(i) 4-[4-(3,4-methylenedioxybenzylamino)benzo[4,5]thieno[2,3-d]-pyrimidin-2-yl]phenylacetic acid;
(j) 4-[4-(3-chloro-4-methoxybenzylamino)benzothieno[2,3-d]-pyrimidin-2-yl]cyclohexanecarboxylic acid.

23. Pharmaceutical formulation according to claim 16, comprising at least 4-[4-(3-chloro-4-methyloxybenzylamino)benzothieno[2,3-d]-pyrimidin-2-yl]cyclohexanecarboxylic acid, ethanolamine salt.

24. Pharmaceutical formulation according to claims 16 to 23, in which the endothelin receptor antagonist is selected from the group consisting of bosentan, tezosentan and sitaxentan.

25. Pharmaceutical formulation according to claims 16 to 23, in which the endothelin receptor antagonist is selected from the group consisting of
a) BMS-193884 (EP 558258),
b) BMS-207940 (Pharmaprojects (13.06.97)),
c) BQ-123 (Exp.Opin.Invest.Drugs, 1997, 6, No.5, 475487),
d) SB-209670 (Exp.Opin.Invest.Drugs, 1997, 6, No.5, 475-487),
e) SB-217242 (Exp.Opin.Invest.Drugs, 1997, 6, No.5, 475487),
f) SB-209598 (Trends in Pharmacol. Sci., 17, 177-81,1996),
g) TAK-044 (Exp.Opin.Invest.Drugs, 1997, 6, No.5, 475487),
h) Bosentan (Trends in Pharmacol. Sci., 18, 408-12, 1997),
i) PD-156707 (J.Med.Chem., 40, No.7,1063-74,1997),
j) L-749329 (Bioorg.Med.Chem.Lett., 7, No.3, 275-280,1997),
k) L-754142 (Exp.Opin.Invest.Drugs, 1997, 6, No.5, 475487),
l) ABT-627 (J.Med.Chem., 40, No.20, 3217-27,1997),
m) A-127772 (J.Med.Chem., 39, No.5,1039-1048,1996),
n) A-206377 (213th American Chemical Society National Meeting, San Francisco, Calif., USA, 13-17 April 1997, Poster, MEDI 193),
o) A-182086 (J.Med.Chem., 40, No.20, 3217-27, 1997),
p) EMD-93246 (211 American Chemical Society National Meeting, New Orleans, USA, 1996, Poster, MEDI 143),
q) EMD-122801 (Bioorg.Med.Chem.Lett., 8, No.1, 17-22,1998),
r) ZD-1611 (Trends in Pharmacol. Sci., 18, 408-12, 1997),
s) AC-610612 (R&D Focus Drug News (18.05.98)),
t) T-0201 (70^{th} Annual Meeting of the Japanese Pharmacological Society, Chiba, Japan, 22-15 March 1997, Lecture, 0-133),
u) J-104132 (R&D Focus Drug News (15.12.97)),

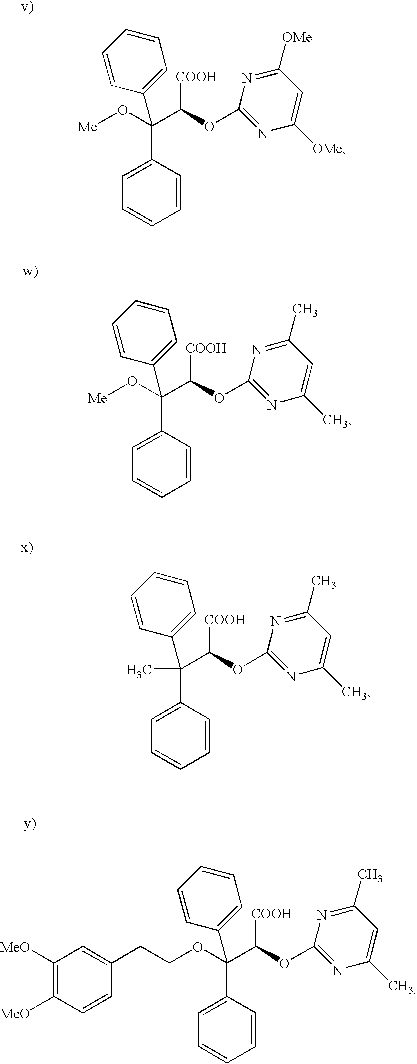

26. Pharmaceutical formulation according to claims 16 to 23, in which the endothelin receptor antagonist is selected from
a) the compounds of the formula I described in EP 0733626 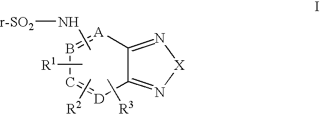
in which
-A=B-C=D- is a —CH═CH—CH═CH— group in which 1 or 2 CH has (have) been replaced by N.
Ar is Ph or naphthyl, each of which is unsubstituted or monosubstituted, disubstituted or trisubstituted by H, Hal, A, alkenyl having up to 6 carbon atoms, Ph, OPh, NO₂, NR⁴R⁵, NHCOR⁴, CF₃, OCF₃, CN, OR⁴, COOR⁴, (CH₂),COORW, (CH₂),NR⁴R⁵, —N═C═O or NHCONR⁴R⁵,
R¹, R²
and R³ are each, independently of one another, absent, H, Hal, A, CF₃, NO₂, NR⁴R⁵, CN, COOR⁴, NHCOR⁴,
R⁴ and R⁵ are each, independently of one another, H or A, or together are alternatively —CH₂—(CH₂), —CH₂—,
A is alkyl having from 1 to 6 carbon atoms,
Ph is phenyl,
X is O or S,
Hal is F, Cl, Br or 1,
n is 1, 2 or 3,
and their salts, with the exception of
4-methyl-N-(2,1,3-benzothiadiazol-4-yl)benzenesulfonamide,
4-methyl-N-(2,1,3-benzothiadiazol-5-yl)benzenesulfonamide, 4-nitro-N-(2,1,3-benzothiadiazol-4-yl)benzenesulfonamide, 4-nitro-N-(2, 1,3-benzothiadiazol-5-yl)benzenesulfonamide, 4-amino-N-(2,1,3-benzo-thiadiazol-4-yl)benzenesulfonamide and 4-amino-N-(2,1,3-benzothia-diazol-5-yl)benzenesulfonamide;
b) the compounds of the formula I described in EP 0733626 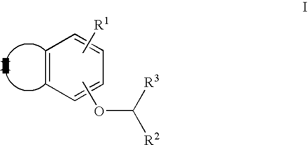
in which
X is a saturated, partially unsaturated or completely unsaturated 3- to 4-membered alkylene chain, in which from 1 to 3 carbon atoms may be replaced by N and/or from 1 to 2 carbon atoms may be replaced by 1-20 atoms and/or 1-2 S atoms, but where at most up to 3 carbon atoms may be replaced and where, in addition, a single, double or triple substitution of the alkylene chain and/or of a nitrogen located therein by A, R¹ and/or NR⁴R⁴ may occur, and where furthermore one CH₂ group in the alkylene chain may also be replaced by a C═O group,
A is alkyl having 1-6 carbon atoms, in which one or two CH₂ groups may be replaced by O or S atoms or by —CR⁴═CR⁴′-groups and in addition 1-7H atoms may be replaced by F,
R¹ is H or A,
R² is COOR⁴, CN, 1H-tetrazol-5-yl or CONHSO₂R⁸,
R³ is Ar,
R⁴ and R^{4′} are each, independently of one another, H, alkyl having from 1 to 6 carbon atoms or benzyl,
Ar is phenyl or naphthyl, each of which is unsubstituted or monosubstituted, disubstituted or trisubstituted by R⁵, R⁶ or R⁷, or is a 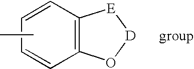
which is unsubstituted or monosubstituted or disubstituted in the phenyl part by R⁵ or R⁵,
R⁵, R⁶
and R⁷ are each, independently of one another, R⁴, OR⁴, Hal, CF₃, OCF₃, OCHF₂, OCH₂F, NO₂, NR⁴R⁴, NHCOR⁴, CN,NHSO₂R⁴, COOR⁴, COR⁴, CONHSO₂R⁴, O(CH₂)R²,
OPh, O(CH₂),OR⁴ or S(O)ₘR⁴,
R⁸ is phenyl or naphthyl, each of which is unsubstituted or monosubstituted, disubstituted or trisubstituted by A, OR¹, NR⁴R⁴ or Hal,
E is CH₂ or O,
D is carbonyl or [C(R⁴R⁴)]ₙ,
Hal is F, Cl, Br or I,
m is 0, 1 or 2,
n is 1 or 2,
and their salts;
c) the compounds of the formula I described in EP 0755934 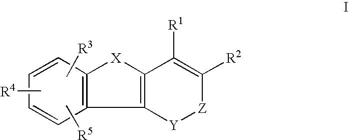
in which
—Y-Z- is —NR⁷—CO—, —N═C(OR⁷)— or —N═CR⁸—,
R¹ is Ar,
R² is COOR⁶, CN, 1H-tetrazol-5-yl or CONHSO₂Ar,
R³, R⁴
and R⁵ are each, independently of one another, R⁶, OR⁶, S(O)ₘR⁸, Hal, NO₂, NR⁶R^{6′}, NHCOR⁶, NHSO₂R⁶, OCOR⁶, COOR⁶ or CN,
R⁶ and R^{6′} are each, independently of one another, H, alkyl having from 1 to 6 carbon atoms, benzyl or phenyl,
R⁷ is (CH₂)nAr,
R⁸ is Ar or OAr,
Ar is phenyl which is unsubstituted or monosubstituted, disubstituted or trisubstituted by R⁹, R¹⁰ or R^{1′}, or is unsubstituted naphthyl or a 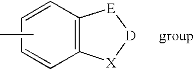
which is unsubstituted or monosubstituted or disubstituted in the phenyl part by R⁹ or R¹⁰, or is a 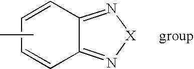
which is unsubstituted or monosubstituted or disubstituted in the cyclohexadienyl part by R⁹ or R¹⁰,
R⁹, R¹⁰
and R¹¹ are each, independently of one another, R⁶, OR⁶, Hal, CF₃, OCF₃, OCHF₂, OCH₂F, NO₂, NR⁶R^{6′}, NHCOR⁶, CN, NHSO₂R⁶, COORW, COR⁶, CONHSO₂Ar, O(CH₂),R², O(CH₂),OR⁶ or S(O)ₘR⁶,
E is CH₂, S or O,
D is carbonyl or [C(R⁶R⁶)])ₙ,
Hal is F, Cl, Br or I,
X is O or S,
m is 0, 1 or 2,
n is 1 or 2,
and their salts;
d) the compounds of the formula I described in EP 0757039 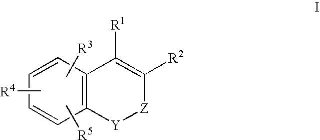
in which —Y-Z- is —NR⁷—CO—, —N═C(OR⁷)— or —N═CR⁹—,
R¹ is Ar,
R² is COOR⁶, (CH₂)ₙCOOR⁶, CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁴, OCOR⁶, COR⁶, COOR⁸ or CN, where R³ and R⁴ together may alternatively be an O(CH₂),O group,
R⁶ and R^{6′} are each, independently of one another, H, alkyl having from 1 to 6 carbon atoms, benzyl or phenyl,
R⁷ is (CH₂),Ar,
R⁸ is Ar or OAr,
Ar is phenyl which is unsubstituted or monosubstituted, disubstituted or trisubstituted by R⁹, R¹⁰ or R^{10′}, or is unsubstituted naphthyl or a 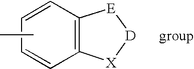
which is unsubstituted or monosubstituted or disubstituted in the phenyl part by R⁹ or R¹⁰, or is a 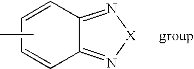
which is unsubstituted or monosubstituted or disubstituted in the cyclohexadienyl part by R⁹ or R¹⁰,
R⁹, R¹⁰
and R¹¹ are each, independently of one another, R¹, OR⁶, Hal, CF₃, OCF₃, OCHF₂, OCH₂F, NO₂, NR⁶RW, NHCOR⁶, CN, NHSO₂R⁶, COOR⁸, COR⁸, CONHSO₂Ar, O(CH₂)ₙR², O(CH₂)ₙOR⁶ or S(O)ₘR⁶,
E is CH₂, S or O,
D is carbonyl or [C(R⁶R^{6′})],
X is O or S,
Hal is F, Cl, Br or I,
m is 0, 1 or 2,
n is 1 or 2,
and their salts;
e) the compounds of the formula I described in EP 0796250 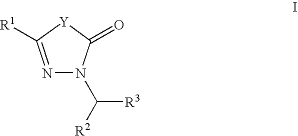
in which
Y is —C(R⁴R⁴)—C(R⁴R⁴)—, —CR⁴═CR⁴′- or —C(R⁴R⁴)—S—,
R¹ is Het, Ar, R³ or R⁴,
R² is Ar or a 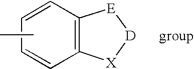
which is unsubstituted or monosubstituted or disubstituted in the phenyl part by A, R³, OR⁴, NH₂, NHA, NA₂, NO₂, CN, Hal, NHCOR⁴, NHSO₂R⁴, COOR⁴, COR⁴, CONHSO₂R³, O(CH₂),R³, OPh, O(CH₂)ₙOR⁴ or S(O)ₘR⁴, or a 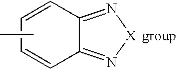
which is unsubstituted or monosubstituted or disubstituted in the cyclohexadienyl part by A, R, OR⁴, NH₂, NHA, NA₂, NO₂, CN, Hal, NHCORW, NHSO₂R⁴, COOR⁴, COR⁴, CONHSO₂R⁶, O(CH₂),R³, OPh, O(CH₂),ORW or S(O)ₘR⁴,
R³ is CN, COOH, COOA, CONHSO₂R⁶ or 1H-tetrazol-5-yl,
R⁴ and R⁴ are each, independently of one another, H, A, or phenyl or benzyl, each of which is unsubstituted or monosubstituted by alkoxy,
R⁵ is A or Ar,
R⁶ is phenyl or naphthyl, each of which is unsubstituted or monosubstituted, disubstituted or trisubstituted by A, OR⁵, NH₂, NHA, NA₂, NO₂, CN or Hal,
A is alkyl having 1-6 carbon atoms, in which one or two CH₂ groups may be replaced by O or S atoms or by —CR⁴═CR⁴′-groups and in addition 1-7H atoms may be replaced by F, or benzyl,
Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂)ₙOR⁴ or S(O)ₘFR⁴,
Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂ and/or carbonyl oxygen,
D is carbonyl or [C(R⁴R^{4′})]ₙ,
E is CH₂, S or O,
Hal is F, Cl, Br or I,
X is O or S,
m is 0, 1 or 2,
n is 1 or 2,
and their salts;
f) the compounds of the formula I described in WO 9719077 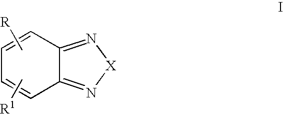
in which
R 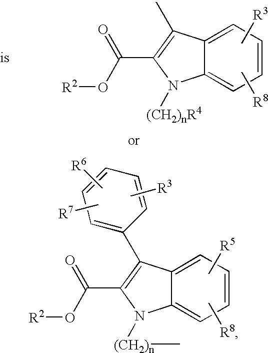
X is O or S,
R¹ is H, Hal, OH, OA, A, alkylene-O-A, NO₂, NH₂, NH-acyl, SO₂NH₂, SO₃-A, SO₂NHA, ON or formyl,
R² is H or A,
R³, R⁵, R⁶
R⁷ and R⁸ are each, independently of one another, H. Hal, OH, OA, O-alkylene-R⁴, A, S-A, NO₂, NH₂, NHA, NA₂, NH-acyl, NHSOA, NHSO₂R⁴, NASOA, NASO₂—R⁴, NH(CO)NH₂, NH(CO)NHA, formyl, NH(CO)NH-phenyl, NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CH₂)ₙOR², CH₂OH or CH₂OA,
R³ and R³ together are alternatively —O—CH₂—O—O—CH₂—CH₂—O—, —O—CH₂—CH₂—, —O—CF₂—O— or —O—CF₂—CF₂—O—,
R⁴ is phenyl which is unsubstituted or monosubstituted or polysubstituted by R³ and/or R⁶,
A is alkyl having 1-6 carbon atoms,
Hal is fluorine, chlorine, bromine or iodine,
n is 1 or 2,
and their salts;
g) the compounds of the formula I described in WO 9730982 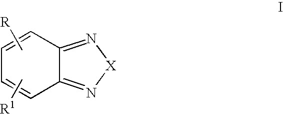
in which
R 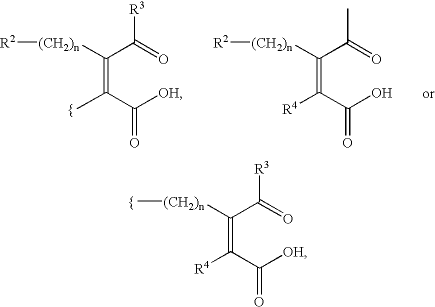
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by Hal, OH, OA, O-alkylene-R⁵, A, S-A, SOA, SO₂A, SOR⁵, SO₂R⁵, NO₂, NH₂, NHA, NA₂, NH-acyl, NHSO₂A, NHSO₂R⁵, NASO₂A, NASO₂-Rr, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂)ₙCOOH, O(CHO,OH, O(CH₂)ₙOA, CH₂OH, CH₂OA, COOH, COOA, CH₂COOH or CH₂COOA, 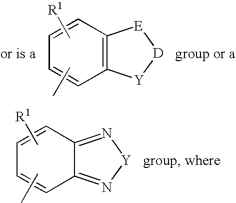
R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶=CR^{6′} groups and/or 1-7H atoms may be replaced by F,
D is carbonyl or [C(R⁶R⁶)]ₘ,
E is CH₂, S or O,
Y is O or S, W⁶ and R⁶ are each, independently of one another, H, F or A,
Hal is fluorine, chlorine, bromine or iodine,
n is 1 or 2, and
m is 1 or 2,
or a tautomeric cyclised form, and the (E)-isomers and the salts of all isomers;
h) the compounds of the formula I described in WO 9730996 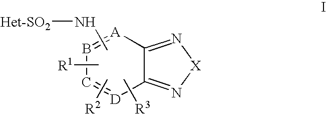
in which
-A=B-C=D- is a —CH═CH—CH═CH— group, in which, in addition, 1 or 2 CH may be replaced by N,
Het is a monocyclic or bicyclic, saturated, unsaturated or aromatic heterocyclic radical having from 1 to 4 N, O and/or S atoms which is unsubstituted or substituted by -Z-R⁶,
R¹, R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—, _{R}⁶ is a phenyl radical, benzothiadiazol-5-yl or benzoxa-diazol-5-yl radical, each of which is unsubstituted or monosubstituted, disubstituted or trisubstituted by R⁷, R⁸ and/or R¹,
R¹, R⁸
and R⁹ are each, independently of one another, A, O-A, CN, COOH, COOA, Hal, formyl, —CO-A, and R⁷ and R⁸ are alternatively —O— (CH₂)ₘ—O—,
A is alkyl having from 1 to 6 carbon atoms,
X is O or S,
Z is —CO—, —CONH—, —CO—(CH₂)ₙ—, —CH═CH—, —(CH₂)ₙ—CONHCO—, —NHCONH—, —NHCOO—, —O—CONH—, —CO—O— or —O—CO—,
Hal is F, Cl, Br or I,
m is 1 or 2, and
n is 1, 2 or 3,
and their salts;
i) the compounds of the formula I described in DE 19609597 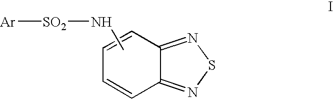
in which
Ar is naphthyl which is monosubstituted by NH₂, NHA or NA₂, and
A is alkyl having from 1 to 6 carbon atoms, and their physiologically acceptable salts;
j) the compounds of the formula I described in DE 19612101 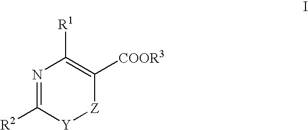
in which
—Y-Z- is —NR⁴—CO or —N═CR⁵—,
R¹ is Ar,
R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by R³, OR³ or Hal,
R³ and R^{3′} are each, independently of one another, H, alkyl having 1-6 carbon atoms or benzyl,
R⁴ is CH₂Ar,
R⁵ is OCH₂Ar,
Ar is phenyl which is unsubstituted or monosubstituted, disubstituted or trisubstituted by R⁶, R⁷ or R⁸, or a 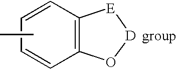
which is unsubstituted or monosubstituted in the phenyl part by R⁶, or a 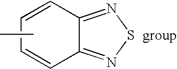
which is unsubstituted or monosubstituted in the cyclo-hexadienyl part by R⁶,
E is CH₂ or O,
D is carbonyl or (CH₂)ₙ,
E and D together are alternatively CH═CR⁹,
R⁶, R^{6′} are each, independently of one another, R³, OR³ or Hal,
R⁷ is R³, OR³, Hal, NO₂, NH₂, NHR³, NR³R³, NHCOR³, COOR³, O(CH₂),R³ or O(CH₂)ₙOR³,
R³ is Ph which is unsubstituted or monosubstituted, disubstituted or trisubstituted by R³, OR³, Hal, NO₂, NH₂, NHR⁶, NR⁶R⁶′, NHCOR³ or COOR³,
R⁹ is H, OH, CH₂OH or COOR³,
Hal is F, Cl, Br or I, Ph is phenyl,
m is 0 or 1,
n is 0 or 2,
and their salts;
k) the compounds of the formula I described in WO 9827091 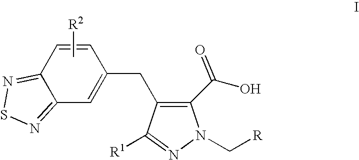
in which
R is phenyl which is unsubstituted or monosubstituted, disubstituted or trisubstituted by R³, R⁴ or R⁵, or 2,1,3-benzothiadiazolyl which is unsubstituted or monosubstituted by R²,
R¹ is A, in which 1-7H atoms may be replaced by F,is —S-A, —O-A, is phenyl or -alkylene-phenyl, each of which is unsubstituted or monosubstituted by R³, or is thienyl which is unsubstituted or monosubstituted by R³,
R² is A, F, Cl, Br or —O-A,
R³, R⁴
and R¹ are each, independently of one another, A, —O-A, —S-A, —O-alkylene-COOH, -alkylene-COOH or COOH,
R³ and R⁴ together are alternatively —O—CH₂—O—, and
A is alkyl having 1-7 carbon atoms, and their salts;
l) the compounds of the formula I described in WO 9827077 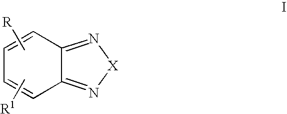
in which
R 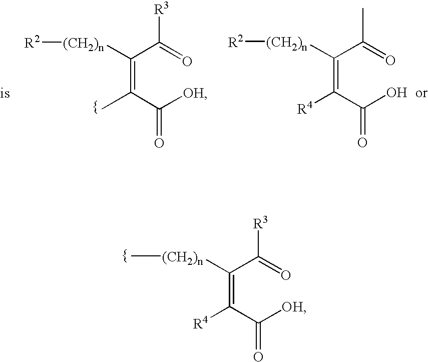
X is O or S,
R¹ is H, Hal, OH, OA, A, alkylene-O-A, NO₂, NH₂, NH-acyl, SO₂NH₂, SO₃-A, SO₂NHA, CN or formyl,
R², R³ and R⁴ are each, independently of one another, a phenyl group which is unsubstituted or monosubstituted or polysubstituted by R⁷, where R² is additionally A or cycloalkyl, or are 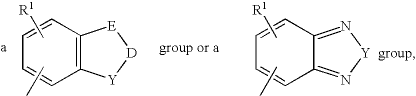
with the proviso that at least one of the radicals R², R³ or
R⁴ is an R⁸ radical which is unsubstituted or monosubstituted or polysubstituted by R⁷,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂),COOA, O(CH₂)ₙ,COOH, O(CHONOH, O(CH₂),OA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶=CR^{6′} groups and/or 1-7H atoms may be replaced by F,
D is carbonyl or [C(R⁶R⁶)]ₘ,
E is CH₂,S or O,
Y is O or S,
R⁶ and R^{6′} are each, independently of one another, H, F or A,
R⁷ is Hal, OH, OA, O-alkylene-R⁵, A, S-A, S-OA, SO₂A, S—OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂),COOA, O(CH₂)ₙCOOH, O(CH₂),OH, O(CH₂),OA, CH₂OH, CH₂OA, COOH, COOA, CH₂COOH or CH₂COOA,
R⁸ is a 5-7-membered heterocyclic radical having 1-4 N, O and/or S atoms or is 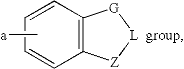
G and Z are each, independently of one another, —CH═, N, O or S,
L is —CH═, —CH═CH— or —CH₂—CH₂—CH₂—,
Hal is fluorine, chlorine, bromine or iodine,
n is 0, 1 or 2, and
m is 1 or 2,
or a tautomeric cyclised form, and the (E)-isomers and the salts of all isomers;
m) the compounds of the formula I described in WO 9841515 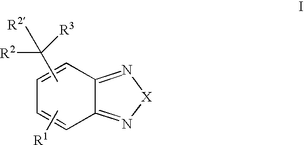
in which
X is O or S,
R¹ is H, Hal, OH, OA, A, NO₂, NH₂, NHA, NAA′, NHCOR⁴, NHCOR⁶, NHSO₂R⁶, NHSO₂R⁶, S(O)ₘR⁸, SO₃H, SO₂NR⁴R^{4′} or formyl,
R² and R^{2′} are each, independently of one another, A, (CH₂)ₙAr, (CH₂)ₙHet, CH₂COAr, CH₂COHet or OAr,
R^{2′} is additionally also H,
R³ is COOR⁴, CN, 1H-tetrazol-5-yl or CONHSO₂R⁵,
R⁴ and R⁴ are each, independently of one another, H or A,
R⁵ is A or Ar,
R⁶ is phenyl or naphthyl, each of which is unsubstituted or monosubstituted, disubstituted or trisubstituted by A, NH₂, NHA, NA′, NO₂, CN or Hal,
R⁷ and R^{7′} are each, independently of one another, H or alkyl having 1-6 carbon atoms,
A and A′ are each, independently of one another, alkyl having 1-6 carbon atoms, in which one or two CH₂ groups may be replaced by O or S atoms or by —CR⁷═CR groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁶, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂),OR⁴, SO₃H, SO₂NR⁴R⁴′, S(O)ₘR⁸ or S(O)ₘR⁴,
Het is a monocyclic or bicyclic, saturated, unsaturated or aromatic heterocyclic radical having 1-4 N, O and/or S atoms, bonded via N or C, which may be unsubstituted or monosubstituted, disubstituted or trisubstituted by
Hal, A, R³, NH₂, NHA, NAA′, NO₂ and/or ═O,
Hal is fluorine, chlorine, bromine or iodine,
m is 0, 1 or 2, and
n is 1 or 2,
where, if R² is CH₂COAr and R^{2′} is H, R³ is not COOA, and salts thereof;
n) the compounds of the formula I described in WO 9841521 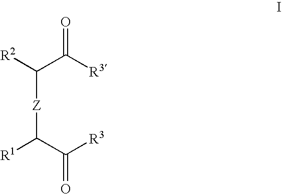
in which
Z is a single or double bond,
R¹ is a 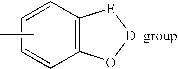
which is unsubstituted or monosubstituted in the phenyl part by R⁷, or is a 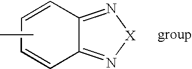
which is unsubstituted or monosubstituted in the cyclohexadienyl part by R⁷,
R² is A, Ar-(CH₂)ₘ, cycloalkyl-(CH₂)ₘ, Het-(CH₂)ₘ or R¹—(CH₂),
R³ and R^{3′} are each, independently of one another, OR⁴, NHSO₂R⁵, NH₂, NHA or NAA′,
R³ and R^{3′} together are alternatively —O—, forming a cyclic anhydride,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ is A, COOR⁴, CN, 1H-tetrazol-5-yl, CONHSO₂R⁶, Hal, OR⁴, NO₂, NH₂, NHA, NAA′, NHCOR⁴, NHCOR^{4′}, NHSO₂R⁴, NHSO₂RG, S(O)ₖR⁴, S(O)ₖRe, SO₂NR⁴R⁴ or formyl,
R⁵ and R^{5′} are each, independently of one another, H or alkyl having 1-6 carbon atoms,
E is CH₂ or O,
D is carbonyl or (CR⁴R^{4′})ₙ,
E and D together are alternatively CR⁴═R⁴,
X is S or O,
A and A′ are each, independently of one another, alkyl having 1-6 carbon atoms, in which one or two CH₂ groups may be replaced by O or S atoms or by —CR⁸═CR^{8′—}groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂ CN, Hal, NHCOR⁴, NHCORE, NHSO₂R⁴, NHSO₂R⁶, COOR⁴, OPh, CONH₂, CONHA, CONAA′, COR⁴, CONHSO₂R⁴, CONHSO₂R⁶, O(CH₂),COOR⁴, O(CH₂)ₙOR⁴, SO₂NR⁴R⁴, S(O)ₖR⁶ or S(O)KR⁴,
Het is a monocyclic or bicyclic, saturated, unsaturated or aromatic heterocyclic radical having 1-4 N, O and/or S atoms, bonded via N or C, which may be unsubstituted, monosubstituted or disubstituted or trisubstituted by Hal, A, COOR⁴, CN, 1H-tetrazol-5-yl, CONHSO₂R⁵, NH₂, NHA, NAA′, NO₂ and/or ═O,
Hal is fluorine, chlorine, bromine or iodine,
k is 0, 1 or 2,
m is 0, 1 or 2, and
n is 1 or 2,
and the (Z)- and (E)-isomers and the salts of all isomers;
o) the compounds of the formula I described in WO 9842702 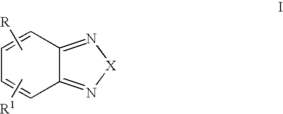
in which
R 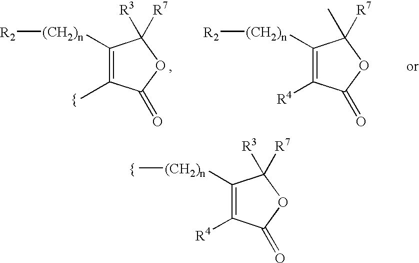
X and Y are each, independently of one another, O or S,
R¹ is H, OH, OA, A alkylene-O-A, NO₂, NH₂, NH-acyl, SO₂NH₂, SO₂-A, SO₂NHA, CN or formyl,
R², R³
and R are each, independently of one another, 2 r Sr, which is unsubstituted or monosubstituted or polysubstituted by Hal, OH, OA, O-alkylene-R⁵, A, S-A, S-OA, SO₂A, 5-OR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH , 1-pyrrolidinyl-CONH, O(CH₂)ₙCOOAf O(OH)COOH, O(CH₂)ₙOH, O(CHOA, CH₂OH, CH₂OA, COOH, COOA, CH₂COOH or CH₂COOA, 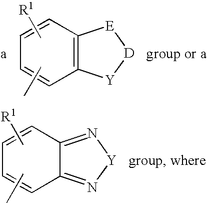
R² is additionally A or cycloalkyl,
R⁶ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CHO,COOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7H atoms may be replaced by F,
D is carbonyl or [C(R⁶R^{6′})]ₘ,
E is CH₂, S or O,
R⁶ and R^{6′} are each, independently of one another, H, F or A,
R⁷ is —O—C(═Y)-NH—R⁸,
R⁸ is alkyl having 1-10 carbon atoms which is unsubstituted or monosubstituted or disubstituted by R′ and in which 1-2 carbon atoms may be replaced by 0 and/or S, and/or may be substituted by ═O, or cycloalkyl, in which 1-2 carbon atoms may be replaced by N, O and/or S,
R⁹ is phenyl which is unsubstituted or monosubstituted or disubstituted by Hal, or is naphthyl, A-O—C(—O)— or Hal,
Hal is fluorine, chlorine, bromine or iodine,
n is 0, 1 or 2, and
m is 1 or 2,
and salts thereof;
p) the compounds of the formula I described in WO 9842709 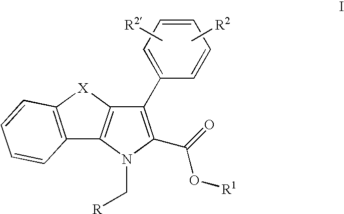
in which
X is N—R³, O or S,
R is 2,1,3-benzothiadiazol-4- or 5-yl or 2,1-benzoisothiazol-5- or 6-yl, each of which is unsubstituted or monosubstituted or disubstituted by R² and/or R²′, or phenyl which is unsubstituted or monosubstituted, disubstituted or trisubstituted by R² and/or R^{2′},
R¹ is H or A,
R² and R^{2′} are each, independently of one another, H, A, OH, OA,
Hal, OCF₃, OCHF₂, —O—CO-A, —O-alkylene-COOR¹, —O-alkylene-CH₂—OR¹, or OCH₂-phenyl or —O—CO-phenyl, each of which is unsubstituted or monosubstituted or disubstituted in the phenyl part by R⁴ and/or R⁴′,
R² and R^{2′} together are alternatively —OCH₂O—, —OCH₂CH₂O— or —OCH₂CH₂—,
R³ is H, A, alkylene-O-A, —CO-OA, or alkylene-phenyl which is unsubstituted or monosubstituted or disubstituted in the phenyl part by RW and/or R^{4′},
R⁴ and R^{4′} are each, independently of one another, H, A, OH, OA,
Hal, COOR¹ or CH₂OR¹,
A is alkyl having 1-6 carbon atoms,
Hal is fluorine, chlorine, bromine or iodine,
and their salts;
q) the compounds of the formula I described in WO 9905132 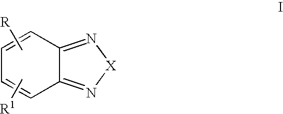
in which
R is 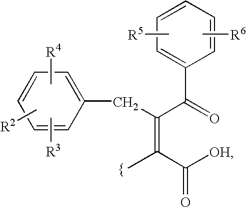
X is O or S,
R¹ is H, Hal, OA or A,
R², R³, R⁵,
and R⁶ are each, independently of one another, H, Hal, A, OA or R⁴,
R⁴ is —O—(CH₂)ₙ—CY,
Cy is cycloalkyl having 3-8 carbon atoms,
A is alkyl having 1-6 carbon atoms, in which one or two CH₂ groups may be replaced by O or S atoms or by —CR⁵—CR⁵— groups and/or 1-7H atoms may be replaced by F,
R⁵ and R^{5″} are each, independently of one another, H, F or A,
Hal is fluorine, chlorine, bromine or iodine,
n is 0, 1 or 2,
or a tautomeric cyclised form, and the (E)-isomers and the salts of all isomers.

27. Pharmaceutical formulation according to one of the preceding claims, comprising one or more excipients and/or assistants.

28. Use of a pharmaceutical preparation according to one of claims 16 to 27 for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

29. Use according to claim 28 for the preparation of a medicament for the treatment of high pulmonary pressure, congestive heart failure (CHF), chronic obstructive pulmonary disease (COPD), cor pulmonale and/or dextrocardiac insufficiency.

30. Set (kit) consisting of separate packs of
(a) an effective amount of 4-[4-(3-chloro-4-methoxybenzylamino)benzothieno[2,3-d]pyrimidin-2-yl]cyclohexanecarboxylic acid and/or physiologically acceptable salts and/or solvates thereof and
(b) an effective amount of an endothelin receptor antagonist.

31. Pharmaceutical formulation comprising at least one compound of the formula I-II 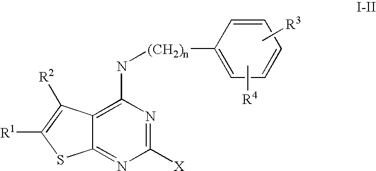
in which
R¹ and R² are each, independently of one another, H, A or Hal, where one of the radicals R¹-or R² is always≠H,
R¹ and R² together are alternatively alkylene having 3-5 carbon atoms,
R³ and R⁴ are each, independently of one another, H, A, OH, OA or Hal,
R³ and R⁴ together are alternatively alkylene having 3-5 carbon atoms, —O—CH₂—CH₂—, —O—CH₂—O— or —O—CH₂—CH₂—O—,
X is R¹ or R⁶, each of which is monosubstituted by R⁷,
R⁵ is linear or branched alkylene having 1-10 carbon atoms, in which one or two CH₂ groups may be replaced by —CH═CH— groups, or —C₂H₄(CHO₂)ₘ,
R⁶ is cycloalkylalkylene having 6-12 carbon atoms,
R⁷ is COOH, COOA, CONH₂, CONHA, CON(A)₂ or CN,
A is alkyl having from 1 to 6 carbon atoms,
Hal is F, Cl, Br or I,
m is 1 or 2, and
n is 0, 1, 2 or 3,
and/or physiologically acceptable salts and/or solvates thereof and at least one endothelin receptor antagonist.

32. Pharmaceutical formulation according to claim 31, comprising at least one compound of the formula I-II according to claim 31 in which
X is R⁵ or R^{5″}, each of which is substituted by COOH or COOA.

33. Pharmaceutical formulation according to claim 31, comprising at least one compound of the formula I-II according to claim 31 in which
R¹ and R² are each, independently of one another, H, A or Hal, where at least one of the radicals R¹ and R² is always≠H.
R³ and R⁴ together are alkylene having 3-5 carbon atoms, —O—CH₂—CH₂—, —O—CH₂—O— or —O—CH₂—CH₂—O,
X is R¹ or R⁶, each of which is substituted by COOH or COOA.

34. Pharmaceutical formulation according to claim 31, comprising at least one compound of the formula I-II according to claim 31 in which
R¹ and R² are each, independently of one another, H, A or Hal, where at least one of the radicals R¹ and R² is always
R³ and R⁴ are each, independently of one another, H, A, OA or Hal,
R³ and R⁴ together are alkylene having 3-5 carbon atoms, —O—CH₂—CH₂—, —O—CH₂—O— or —O—CH₂—CH₂—O,
X is R¹ or RW, each of which is substituted by COOH or COOA,
n is 1 or 2.

35. Pharmaceutical formulation according to claim 31, comprising at least one compound of the formula I-II according to claim 31 in which
R¹ and R² are each, independently of one another, H, A or Hal, where one of the radicals R¹ and R² is always≠H,
R¹ and R² together are alternatively alkylene having 3-5 carbon atoms,
R³ and R⁴ are each, independently of one another, H, A, OA or Hal,
R³ and R⁴ together are alternatively —C—CH₂—C—,
X is R⁵ which is monosubstituted by R⁷,
R⁵ is linear or branched alkylene having 1-10 carbon atoms, or —C₆H₄—CH₂—,
R⁷ is COOH or COOA,
A is alkyl having from 1 to 6 carbon atoms,
Hal is F, Cl, Br or 1,
m is 1, and
n is 1 or 2.

36. Pharmaceutical formulation according to claim 31, comprising at least one compound of the formula I-II according to claim 31 in which
R¹ and R² are each, independently of one another, H, A or Hal, where one of the radicals R¹ and R² is always≠H,
R¹ and R² together are alternatively alkylene having 3-5 carbon atoms,
R³ and R⁴ are each, independently of one another, H, A, OH, OA or Hal,
R³ and R⁴ together are alternatively —O—CH₂—O—,
X is R⁵ which is monosubstituted by R⁷,
R⁵ is linear or branched alkylene having 1-10 carbon atoms, or —C₆H₄—CH₂—,
R⁷ is COOH or COOA,
A is alkyl having from 1 to 6 carbon atoms,
Hal is F, Cl, Br or 1,
m is 1, and
n is 1 or 2.

37. Pharmaceutical formulation according to claim 31, comprising at least one compound of the formula I-II according to claim 31, selected from the group consisting of
(a) 3-[4-(3-chloro-4-methoxybenzylamino)-5,6,7,8-tetrahydro-[1]-benzothieno[2,3-d]pyrimidin-2-yl]propionic acid;
(b) 4-[4-(3,4-methylenedioxybenzylamino)-5,6,7,8-tetrahydro-[1]-benzothieno[2,3-d]pyrimidin-2-yl]butyric acid;
(c) 7-[4-(3,4-methylenedioxybenzylamino)-5,6,7,8-tetrahydro-[1]-benzothieno[2,3-d3pyrimidin-2-yl]heptanoic acid;
(d) 7-[4-(3-chloro-4-methoxybenzylamino)-5,6,7,8-tetrahydro-[1]-benzothieno[2,3-d]pyrimidin-2-yl]heptanoic acid;
(e) 5-[4-(3-chloro-4-methoxybenzylamino)-5,6,7,8-tetrahydro-[1]-benzothieno[2,3]pyrimidin-2-yl]valeric acid;
(e) 5-[4-(3-chloro-4-methoxybenzylamino)-6-methylthieno[2,3d]-pyrimidin-2-yl]valeric acid;
(g) 4-[4-(3-chloro-4-methoxybenzylamino)-6-methylthieno[2,3-d]-pyrimidin-2-yl]butyric acid;
(h) 4-[4-(3,4-methylenedioxybenzylamino)-6-methylthieno[2,3-d]-pyrimidin-2-yl]butyric acid;
(i) 2-{4-[4-(3-chloro-4-methoxybenzylamino)-5,6,7,8-tetrahydro-[1]-benzothieno[2,3-d]pyrimidin-2-yl]cyclohexyl-1-yl}acetic acid;
(k) 5-[4-(3,4-methylenedioxybenzylamino)-6-methylthieno[2,3-d]-pyrimidin-2-yl]valeric acid.

38. Pharmaceutical formulation according to claim 31, comprising 5-[4-(3-chloro-4-methoxybenzylamino)-5,6,7,8-tetrahydro-[1]-benzothieno-[2,3-d]pyrimidin-2-yl]valeric acid, ethanolamine salt.

39. Pharmaceutical formulation according to claims 31 to 38, in which the endothelin receptor antagonist is selected from the group consisting of bosentan, tezosentan and sitaxentan.

40. Pharmaceutical formulation according to claims 31 to 38, in which the endothelin receptor antagonist is selected from the group consisting of
a) BMS-193884 (EP 558258),
b) BMS-207940 (Pharmaprojects (13.06.97)),
c) BQ-123 (Exp.Opin.Invest.Drugs, 1997, 6, No.5, 475487),
d) SB-209670 (Exp.Opin.Invest.Drugs, 1997, 6, No.5, 475487),
e) SB-217242 (Exp.Opin.Invest.Drugs, 1997, 6, No.5, 475487),
f) SB-209598 (Trends in Pharmacol. Sci., 17, 177-81,1996),
g) TAK-044 (Exp.Opin.Invest.Drugs, 1997, 6, No.5, 475-487),
h) Bosentan (Trends in Pharmacol. Sci., 18, 408-12, 1997),
i) PD-156707 (J.Med.Chem., 40, No.7, 1063-74, 1997),
j) L-749329 (Bioorg.Med.Chem.Lett., 7, No.3, 275-280,1997),
k) L-754142 (Exp.Opin.Invest.Drugs, 1997, 6, No.5, 475487),
l) ABT-627 (J.Med.Chem., 40, No.20, 3217-27,1997),
m) A-127772 (J.Med.Chem., 39, No.5, 1039-1048, 1996),
n) A-206377 (2131 American Chemical Society National Meeting, San Francisco, Calif., USA, 13-17 April 1997, Poster, MEDI 193),
o) A-182086 (J. Med.Chem., 40, No.20, 3217-27, 1997),
p) EMD-93246 (211^{th} American Chemical Society National Meeting, New Orleans, USA, 1996, Poster, MEDI 143),
q) EMD-122801 (Bioorg.Med.Chem.Lett., 8, No.1, 17-22, 1998),
r) ZD-1611 (Trends in Pharmacol. Sci., 18, 408-12, 1997),
s) AC-610612 (R&D Focus Drug News (18.05.98)),
t) T-0201 (7CP Annual Meeting of the Japanese Pharmacological Society, Chiba, Japan, 22-15 March 1997, Lecture, 0-133),
u) J-104132 (R&D Focus Drug News (15.12.97)),

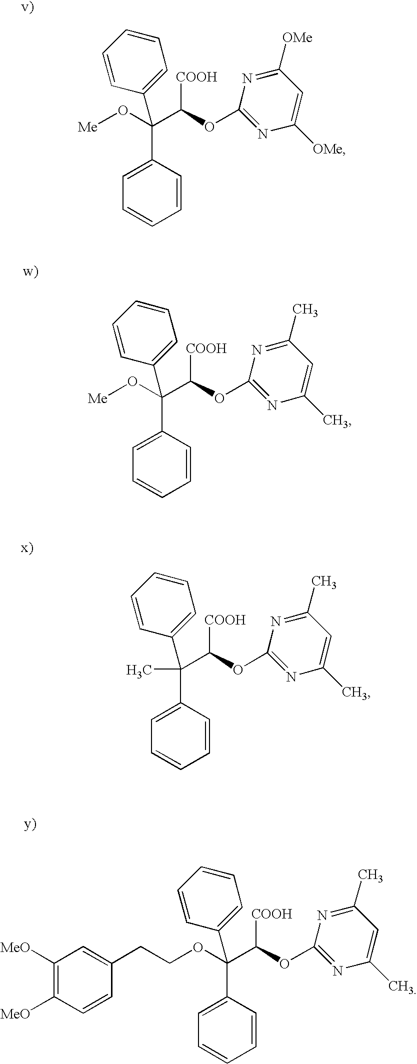

41. Pharmaceutical formulation according to claims 31 to 38, In which the endothelin receptor antagonist is selected from
a) the compounds of the formula I described in EP 0733626 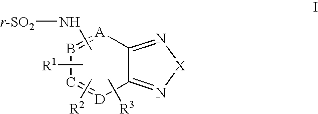
in which
-A=B-C=D- is a —CH═CH—CH═CH— group in which 1 or 2 CH has (have) been replaced by N,
Ar is Ph or naphthyl, each of which is unsubstituted or monosubstituted, disubstituted or trisubstituted by H,
Hal, A, alkenyl having up to 6 carbon atoms, Ph, OPh, NO₂, NR⁴R⁵, NHCOR⁴, CF₃, OCF₃, CN, OR⁴, COOR⁴, (CH₂),COOR⁴, (CH₂),NR⁴R⁵, —N═C═O or NHCONR⁴R⁵,
R¹, R²
and R³ breach, independently of one another, absent, H, Hal, A, CF₃, NO₂, NR⁴R⁵, CN, COOR⁴, NHCOR⁴,
R⁴ and R⁵ are each, independently of one another, H or A, or together are alternatively —CH₂—(CH₂)ₙ—CH₂—,
A is alkyl having from 1 to 6 carbon atoms,
Ph is phenyl,
X is O or S,
Hal is F, Cl, Br or I,
n is 1, 2 or 3,
and their salts, with the exception of
4-methyl-N-(2,1,3-benzothiadiazol-4-yl)benzenesulfonamide, 4-methyl-N-(2,1,3-benzothiadiazol-5-yl)benzenesulfonamide, 4-nitro-N-(2,1,3-benzothiadiazol-4-yl)benzenesulfonamide, 4-nitro-N-(2, 1,3-benzothiadiazol-5-yl)benzenesulfonamide, 4-amino-N-(2,1,3-benzo-thiadiazol-4-yl)benzenesulfonamide and 4-amino-N-(2,1,3-benzothiadiazol-5-yl)benzenesulfonamide;
b) the compounds of the formula I described in EP 0733626 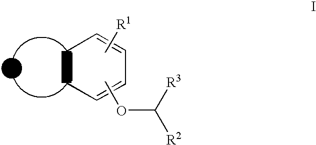
in which
X is a saturated, partially unsaturated or completely unsaturated 3- to 4-membered alkylene chain, in which from 1 to 3 carbon atoms may be replaced by N and/or from 1 to 2 carbon atoms may be replaced by 1-2 O atoms and/or 1-2 S atoms, but where at most up to 3 carbon atoms may be replaced and where, in addition, a single, double or triple substitution of the alkylene chain and/or of a nitrogen located therein by A, R⁸ and/or NR⁴R^{4′} may occur, and where furthermore one CH₂ group in the alkylene chain may also be replaced by a C═O group,
A is alkyl having 1-6 carbon atoms, in which one or two CH₂ groups may be replaced by O or S atoms or by —CR⁴═CR⁴— groups and in addition 1-7H atoms may be replaced by F,
R¹ is H or A,
R² is COOR⁴, CN, 1H-tetrazol-5-yl or CONHSO₂R⁸,
R³ is Ar,
R⁴ and R^{4′} are each, independently of one another, H, alkyl having from 1 to 6 carbon atoms or benzyl,
Ar is phenyl or naphthyl, each of which is unsubstituted or monosubstituted, disubstituted or trisubstituted by R⁵, R⁶ or R⁷, or is a 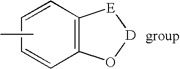
which is unsubstituted or monosubstituted or disubstituted in the phenyl part by R⁵ or R⁶,
R⁵, R⁶
and R⁷ are each, independently of one another, R⁴, OR⁴, Hal, CF₃, OCF₃, OCHF₂, OCH₂F, NO₂, NR⁴R, NHCOR⁴, CN₂NHSO₂R⁴, COOR⁴, COR⁴, CONHSO₂R⁸, O(CH₂)ₙR², OPh, O(CH₂)ₙOR⁴ or S(O)ₘR⁴,
R⁸ is phenyl or naphthyl, each of which is unsubstituted or monosubstituted, disubstituted or trisubstituted by A, OR¹, NR⁴R^{4′} or Hal,
E is CH₂ or O,
D is carbonyl or (C(R⁴R⁴)]ₙ,
Hal is F, Cl, Br or I,
m is 0, 1 or 2,
n is 1 or 2,
and their salts;
c) the compounds of the formula I described in EP 0755934 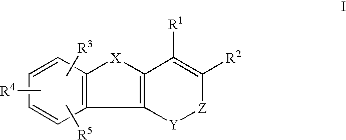
in which
—Y-Z- is —NR⁷—CO-_-N═C(OR⁷)— or —N═CR⁸—,
R¹ is Ar,
R² is COOR⁶, CN₂H-tetrazol-5-yl or CONHSO₂Ar,
R³, R⁴ and R⁵ are each, independently of one another, R⁶, OR⁶, S(O)ₘR⁶, Hal, NO₂, NR⁶R⁸, NHCOR⁶, NHSO₂R⁶, OCOR⁶, COOR¹ or CN,
R⁶ and R^{6′} are each, independently of one another, H, alkyl having from 1 to 6 carbon atoms, benzyl or phenyl,
R⁷ is (CH₂),Ar,
R⁸ is Ar or OAr,
Ar is phenyl which is unsubstituted or monosubstituted, disubstituted or trisubstituted by R⁹, R¹⁰ or R¹¹, or is unsubstituted naphthyl or a 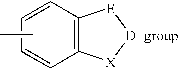
which is unsubstituted or monosubstituted or disubstituted in the phenyl part by R⁹ or R¹⁰, or is a 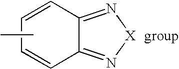
which is unsubstituted or monosubstituted or disubstituted in the cyclohexadienyl part by R⁹ or R¹⁰,
R⁹, R¹⁰
and R¹¹ are each, independently of one another, R⁶, OR⁶, Hal, CF₃, OCF₃, OCHF₂, OCH₂F, NO₂, N⁶R⁶R^{6′}, NHCOR⁸, CN, NHSO₂R⁸, COOR⁸, COR⁶, CONHSO₂Ar, O(CH₂)ₙR², O(CH₂)ₙOR⁶ or S(O)ₘR⁶,
E is CH₂, S or O,
D is carbonyl or [C(R⁶R⁶)]ₙ,
Hal is F, Cl, Br or 1,
X is O or S,
m is 0, 1 or 2,
n is 1 or 2,
and their salts;
d) the compounds of the formula I described in EP 0757039 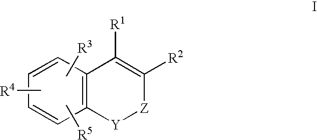
in which —Y-Z- is —NR⁷—CO—, —N═C(OR⁷)— or —N═CR¹—,
R¹ is Ar,
R² is COORB, (CH₂),COOR , CN, 1H-tetrazol-5-yl or CONHSO₂Ar,
R³, R⁴
and R⁵ are each, independently of one another, R⁶, OR⁶, S(O)ₘR⁶, Hal, NO₂, NR⁶R^{6′}, NHCOR⁶, NHSO₂R⁶, OCOR⁶, COR⁶, COOR⁶ or CN, where R³ and R⁴ together may alternatively be an O(CH₂),O group,
R⁶ and R^{6′} are each, independently of one another, H, alkyl having from 1 to 6 carbon atoms, benzyl or phenyl,
R⁷ is (CH₂)nAr,
R⁸ is Ar or OAr,
Ar is phenyl which is unsubstituted or monosubstituted, disubstituted or trisubstituted by R⁹, R¹⁰ or R¹¹, or is unsubstituted naphthyl or a 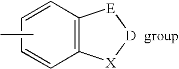
which is unsubstituted or monosubstituted or disubstituted in the phenyl part by R⁹ or R¹⁰, or is a 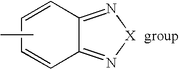
which is unsubstituted or monosubstituted or disubstituted in the cyclohexadienyl part by R⁹ or R¹⁰,
R⁹, R¹⁰
and R¹¹ are each, independently of one another, R, OR^{6′}, Hal, CF₃, OCF₃, OCHF₂, OCH₂F, NO₂, NR⁶R^{6′}, NHCOR⁶, CN, NHSO₂R⁶, COOR⁶, COR⁶, CONHSO₂Ar, O(CH₂)ₙR², O(CH₂)ₙOR⁶ or S(O)ₘR⁶,
E is CH₂, S or O,
D is carbonyl or [C(R⁶R⁶)]ₙ,
X is O or S,
Hal is F, Cl, Br or I,
m is 0, 1 or 2,
n is 1 or 2,
and their salts;
e) the compounds of the formula I described in EP 0796250 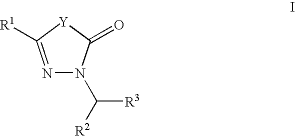
in which
Y is —C(R⁴R^{4′})—C(R⁴R^{4′})—, —CR⁴═CR^{4′} — or —C(R⁴R^{4′})—S—,
R¹ is Het, Ar, R³ or R⁴,
R² is Ar or a 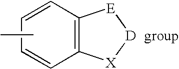
which is unsubstituted or monosubstituted or disubstituted in the phenyl part by A, R³, OR⁴, NH₂, NHA, NA₂, NO₂, CN, Hal, NHCOR⁴, NHSO₂R⁴, COOR⁴, COR⁴, CONHSO₂R⁶, O(CH₂),R³, OPh, O(CH₂)ₙOR⁴ or S(O)ₙR⁴, or a 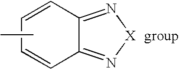
which is unsubstituted or monosubstituted or disubstituted in the cyclohexadienyl part by A, R³, OR⁴, NH₂, NHA, NA₂, NO₂, CN, Hal, NHCOR⁴, NHSO₂R⁴, COOR⁴, COR⁴, CONHSO₂R⁶, O(CH₂)ₙR³, OPh, O(CH₂),OR⁴ or S(O),R,
R³ is CN, COOH, COOA, CONHSO₂R¹ or 1H-tetrazol-5-yl,
R⁴ and R^{4′} are each, independently of one another, H, A, or phenyl or benzyl, each of which is unsubstituted or monosubstituted by alkoxy,
R⁵ is A or Ar,
R⁶ is phenyl or naphthyl, each of which is unsubstituted or monosubstituted, disubstituted or trisubstituted by A, OR⁵, NH₂, NHA, NA₂, NO₂, CN or Hal,
A is alkyl having 1-6 carbon atoms, in which one or two CH₂ groups may be replaced by O or S atoms or by —CR⁴═CR^{4′}— groups and in addition 1-7H atoms may be replaced by F, or benzyl,
Ar is phenyl or naphthyl, each of which is unsubstituted or monosubstituted, disubstituted or trisubstituted by A, OR⁴, NH₂, NHA, NA₂, NO₂, CN, Hal, NHCOR⁴, NHSO₂RC, COOR⁴ COR⁴, CONHSO₂R⁶, O(CH₂)ₙR³, OPh, O(CH₂)ₙOR⁴ or S(O)ₘR⁴,
Het is a monocyclic or bicyclic, saturated, unsaturated or aromatic heterocyclic radical having from 1 to 4 N, O and/or S atoms, bonded via N or C, which may be unsubstituted or monosubstituted, disubstituted or trisubstituted by Hal, A, R³, NH₂, NHA, NA₂, CN, NO₂ and/or carbonyl oxygen,
D is carbonyl or [C(R⁴R⁴)],
E is CH₂, S or O,
Hal is F, Cl, Br or I,
X is O or S,
m is 0, 1 or 2,
n is 1 or 2,
and their salts;
f) the compounds of the formula I described in WO 9719077 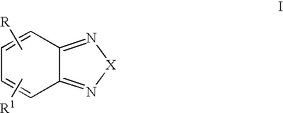
in which
R 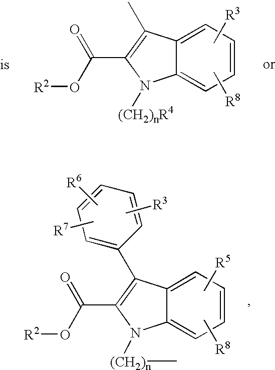
X is O or S,
R¹ is H, Hal, OH, OA, A, alkylene-O-A, NO₂, NH₂, NH-acyl, SO₂NH₂, SO₃-A, SO₂NHA, CN or formyl,
R² is H or A,
R⁵, R⁶, R⁶
R⁷ and R⁸ are each, independently of one another, H, Hal, OH, OA, O-alkylene-R⁴, A, S-A, NO₂, NH₂, NHA, NA₂, NH-acyl, NHSO₂A, NHSO₂R⁴, NASO₂A, NASO₂—R⁴, NH(CO)NH₂, NH(CO)NHA, formyl, NH(CO)NH-phenyl, NHCOOA, NA-acyl, NHR⁴, NHCOOR⁴, NHCOO-benzyl, NHSO₂-benzyl, NHCOO-alkylene-OA, NH(CO)NA₂, N-piperidinyl-CO—NH, N-pyrrolidinyl-CONH, O(CH₂)ₙCOOR², O(CHONOR², CH₂OH or CH₂OA,
R³ and R¹ together are alternatively —O—CH₂—O—, —O—CH₂—CH₂—O—, —O—CH₂—CH₂—, —O—CF₂—O— or —O—CF₂—CF₂—O—,
R⁴ is phenyl which is unsubstituted or monosubstituted or polysubstituted by R³ and/or R⁶,
A is alkyl having 1-6 carbon atoms,
Hal is fluorine, chlorine, bromine or iodine,
n is 1 or 2,
and their salts;
g) the compounds of the formula I described in WO 9730982 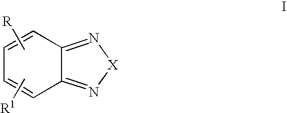
in which
R 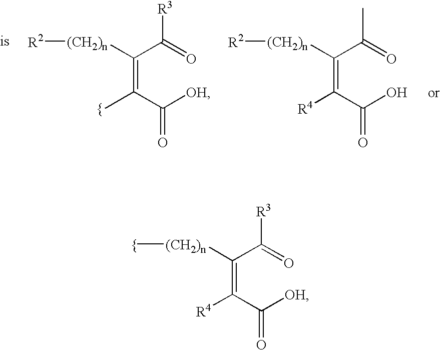
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by Hal, OH, OA, O-alkylene R⁵, A, S-A, SOA, SO₂A, SOR⁵, SO₂R⁵, NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁶, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂),COOA, O(CH₂),COOH, O(CH₂),OH, O(CH₂)ₙOA, CH₂OH, CH₂OA, COOH, COOA, CH₂COOH or CH₂COOA, 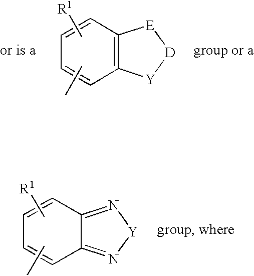
R² is additionally A or cycloalkyl,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂),COOH, O(CH₂)ₙOH, O(CH₂)ₙOA, CH₂OH, CH₂OA, COH₁, COOA, CH₂COOH or CH₂COOA,
A is alkyl having 1-6 carbon atoms, in which one or two CH₂ groups may be replaced by O or S atoms or by —CR⁶═CR^{6′}— groups and/or 1-7H atoms may be replaced by F,
D is carbonyl or [C(R⁶R^{6′})]ₘ,
E is CH₂, S or O,
Y is O or S,
R⁶ and R^{6′} are each, independently of one another, H, F or A,
Hal is fluorine, chlorine, bromine or iodine,
n is 1 or 2, and
m is 1 or 2,
or a tautomeric cyclised form, and the (E)-isomers and the salts of all isomers;
h) the compounds of the formula I described in WO 9730996 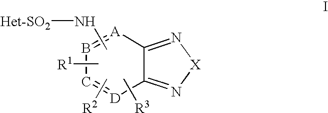
in which
-A=B-C=D- is a —CH═CH—CH—CH— group, in which, in addition, 1 or 2 CH may be replaced by N,
Het is a monocyclic or bicyclic, saturated, unsaturated or aromatic heterocyclic radical having from 1 to 4 N, O and/or S atoms which is unsubstituted or substituted by -Z-R⁶,
R¹,R²
and R³ are each, independently of one another, absent, H, Hal, A, CF₃, NO₂, NR⁴R⁵, CN, COOR⁴ or NHCOR⁴,
R⁴ and R⁵ are each, independently of one another, H or A, or together are alternatively —CH₂—(CH₂)ₙ—CH₂—,
R⁶ is a phenyl radical, benzothiadiazol-5-yl or benzoxadiazol-5-yl radical, each of which is unsubstituted or monosubstituted, disubstituted or trisubstituted by R⁷, Re and/or R¹,
R⁷, R⁸
and R⁹ are each, independently of one another, A, O-A, CN, COOH, COOA, Hal, formyl, —CO-A, and R⁷ and R⁸ are alternatively —O—(CH₂)ₘ—O—,
A is alkyl having from 1 to 6 carbon atoms,
X is O or S,
Z is —CO—, —CONH—, —CO—(CH₂)×n—, —CH═CH—, —(CH₂)ₙ—, —CONHCO—, —NHCONH—, —NHCOO—, —O—CONH—, —CO—O— or —O—CO—,
Hal is F, Cl, Br or I,
m is 1 or 2, and
n is 1, 2 or 3,
and their salts;
i) the compounds of the formula I described in DE 19609597 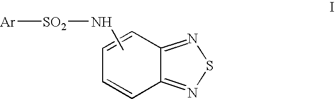
in which
Ar is naphthyl which is monosubstituted by NH₂, NHA or NA₂, and
A is alkyl having from 1 to 6 carbon atoms,
and their physiologically acceptable salts;
j) the compounds of the formula I described in DE 19612101 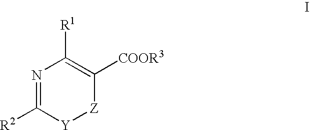
in which
—Y-Z- is —NR⁴—CO or —N═CR⁵—,
R¹ is Ar,
R² is H, alkyl having 1-6 carbon atoms which is unsubstituted or monosubstituted, disubstituted or trisubstituted by OR³ or Hal, or (CH₂)ₘPh or (CH₂)ₘ-cycloalkyl, each of which is unsubstituted or monosubstituted, disubstituted or trisubstituted by R³, OR³ or Hal,
R³ and R^{3′} are each, independently of one another, H, alkyl having 1-6 carbon atoms or benzyl,
R⁴ is CH₂Ar,
R⁵ is OCH₂Ar,
Ar is phenyl which is unsubstituted or monosubstituted, disubstituted or trisubstituted by R⁶, R⁷ or R³, or a 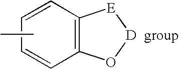
which is unsubstituted or monosubstituted in the phenyl part by R⁶, or a 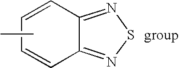
which is unsubstituted or monosubstituted in the cyclohexadienyl part by R⁶⁻;
E is CH₂ or O,
D is carbonyl or (CH₂)ₙ,
E and D together are alternatively CH═CR⁹,
R⁶, R^{6′} are each, independently of one another, R³, OR³ or Hal,
R⁷ is R³, OR³, Hal, NO₂, NH₂, NHR³, NR³R³, NHCOR³, COOR³, O(CH₂)ₙRⁿ or O(CH₂)ₙOR³,
R⁸ is Ph which is unsubstituted or monosubstituted, disubstituted or trisubstituted by R³, OR³, Hal, NO₂, NH₂, NHR⁶, NR⁶R^{6′}, NHCOR³ or COOR³,
R⁹ is H, OH, CH₂OH or COOR³,
Hal is F, Cl, Br or I, Ph is phenyl,
m is 0 or 1,
n is 1 or 2,
and their salts;
k) the compounds of the formula I described in WO 9827091 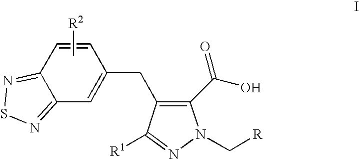
in which
R is phenyl which is unsubstituted or monosubstituted, disubstituted or trisubstituted by R³, R⁴ or R⁶, or 2,1,3-benzothiadiazolyl which is unsubstituted or monosubstituted by R²,
R₁ is A, in which 1-7H atoms may be replaced by F,is —S-A, —O-A, is phenyl or -alkylene-phenyl, each of which is unsubstituted or monosubstituted by R¹, or is thienyl which is unsubstituted or monosubstituted by R³,
R² is A, F, Cl, Br or —O-A,
R³, R⁴
and R¹ are each, independently of one another, A, —O-A, —S-A, —O-alkylene-COOH, -alkylene-COOH or COOH,
R³ and R⁴ together are alternatively —O—CH₂—O—, and
A is alkyl having 1-7 carbon atoms, and their salts;
l) the compounds of the formula I described in WO 9827077 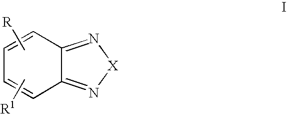
in which
R 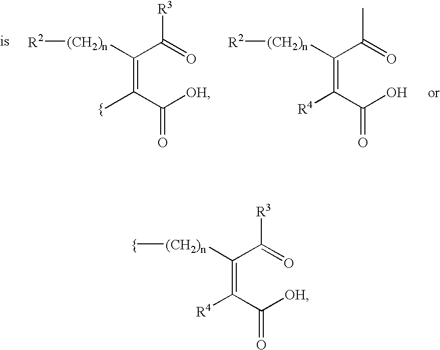
X is O or S,
R¹ is H, Hal, OH, OA, A, alkylene-O-A, NO₂, NH₂, NH-acyl, SO₂NH₂, SO₃-A, SO₂NHA, CN or formyl,
R², R³
and R⁴ are each, independently of one another, a phenyl group which is unsubstituted or monosubstituted or polysubstituted by R⁷, where R² is additionally A or cycloalkyl, or are 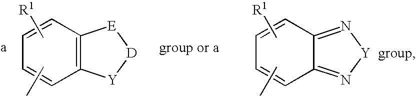
with the proviso that at least one of the radicals R², R³ or
R⁴ is an R⁸ radical which is unsubstituted or monosubstituted or polysubstituted by R⁷,
R⁵ is a phenyl group which is unsubstituted or monosubstituted or polysubstituted by Hal, OH, OA, A, S-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂),OA, CH₂OH, CH₂OA, COOH, COOA, CH₂COOH or CH₂COOA,
A is alkyl having 1-6 carbon atoms, in which one or two CH₂ groups may be replaced by O or S atoms or by —CR^{6′} CR⁶— groups and/or 1-7H atoms may be replaced by F,
D is carbonyl or [C(R⁶R⁶)]ₘ,
E is CH₂, S or O,
Y is O or S,
R⁶ and R^{6′} are each, independently of one another, H, F or A,
R⁷ is Hal, OH, OA, O-alkylene-R⁶, A, S-A, S-OA, SO₂A, S—OR, SO₂R², NO₂, NH₂, NHA, NA₂, NH-acyl, NHSO₂A, NHSO₂R⁵, NASO₂A, NASO₂—R⁵, NH(CO)NH₂, NH(CO)NHA, formyl, NH(CO)NHR⁶, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂)ₙCOOA, O(CH₂),COOH, O(CH₂),OH, O(CH₂),OA, CH₂OH, CH₂OA, COOH, COOA, CH₂COOH or CH₂COOA,
R⁸ is a 5-7-membered heterocyclic radical having 14 N, O and/or S atoms or is 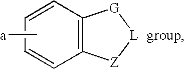
G and Z are each, independently of one another, —CH—, N, O or S,
L is —CH═, —CH═CH— or —CH₂—CH₂—CH₂—,
Hal is fluorine, chlorine, bromine or iodine,
n is 0, 1 or 2, and
m is 1 or 2,
or a tautomeric cyclised form, and the (E)-isomers and the salts of all isomers;
m) the compounds of the formula i described in WO 9841515 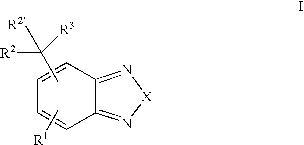
in which
X is O or S,
R¹ is H, Hal, OH, OA, A, NO₂, NH₂, NHA, NAA′, NHCOR⁴, NHCOR⁶, NHSO₂R⁴, NHSO₂R⁶, S(O)ₘR⁶, SO₃H, SO₂NR⁴R^{4′} or formyl,
R² and R³ are each, independently of one another, A, (CH₂),Ar, (CH₂),Het, CH₂COAr, CH₂COHet or OAr,
R^{2′} is additionally also H,
R³ is COOR⁴, CN, ₁H-tetrazol-5-yl or CONHSO₂R⁶,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ and R¹ are each, independently of one another, H or alkyl having 1-6 carbon atoms,
A and A′ are each, independently of one another, alkyl having 1-6 carbon atoms, in which one or two CH₂ groups may be replaced by O or S atoms or by —CR⁷═CR groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCORB, NHSO₂R⁴, NHSO₂R⁴, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₃H, SO₂NR⁴R⁴′, S(O)ₘR⁶ or S(O)ₘR⁴,
Het is a monocyclic or bicyclic, saturated, unsaturated or aromatic heterocyclic radical having 1-4 N, O and/or S atoms, bonded via N or C, which may be unsubstituted or monosubstituted, disubstituted or trisubstituted by
Hal, A, R³, NH₂, NHA, NAA′, NO₂ and/or ═O,
Hal is fluorine, chlorine, bromine or iodine,
m is 0, 1 or 2, and
n is 1 or 2,
where, if R² is CH₂COAr and RZ is H, R³ is not COOA,
and salts thereof;
n) the compounds of the formula I described in WO 9841521 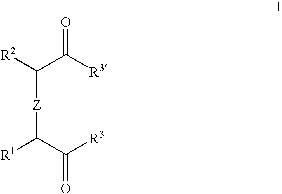
in which
Z is a single or double bond,
R¹ is a 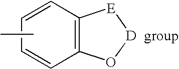
which is unsubstituted or monosubstituted in the phenyl part by R⁷, or is a 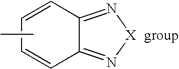
which is unsubstituted or monosubstituted in the cyclohexadienyl part by R¹,
R² is A, Ar-(CH₂)ₘ, cycloalkyl-(CH₂)ₘ, Het-(CH₂)ₘ or R¹—(CH₂)ₘ,
R³ and R³ are each, independently of one another, OR⁴, NHSO₂R⁵, NH₂, NHA or NAA′,
R¹ and R¹ together are alternatively —O—, forming a cyclic anhydride,
R⁴ and R^{4′} are each, independently of one another, H or A,
R⁵ is A or Ar,
R⁶ is phenyl or naphthyl, each of which is unsubstituted or monosubstituted, disubstituted or trisubstituted by A, NH₂, NHA, NAA′, NO₂, CN or Hal,
R⁷ is A, COOR⁴, CN, 1H-tetrazol-5-yl, CONHSO₂R⁵, Hal, OR⁴, NO₂, NH₂, NHA, NAA′, NHCOR⁴, NHCOR⁶, NHSO₂R⁴, NHSO₂R⁶, S(O)ₖR⁴, S(O)ₖR⁴, SO₂NR⁴R⁴ or formyl,
R⁸ and R^{8′} are each, independently of one another, H or alkyl having 1-6 carbon atoms,
E is CH, or O,
D is carbonyl or (CR⁴R^{4′})ₙ,
E and D together are alternatively CR⁴═R^{4′},
X is S or O,
A and A′ are each, independently of one another, alkyl having 1-6 carbon atoms, in which one or two CH₂ groups may be replaced by O or S atoms or by —CR⁸═CR⁸— groups and/or 1-7H atoms may be replaced by F, or benzyl,
Ar is phenyl or naphthyl, each of which is unsubstituted or monosubstituted, disubstituted or trisubstituted by A, OR⁴, NH₂, NHA, NAA′, NO₂, CN, Hal, NHCOR⁴, NHCOR⁸, NHSO₂R⁴, NHSO₂R⁶, COOR⁴, OPh, CONH₂, CONHA, CONAA′, COR⁴, CONHSO₂R⁴, CONHSO₂R⁶, O(CH₂)ₙCOOR⁴, O(CH₂)ₙOR⁴, SO₂NR⁴R⁴′, S(O)ₖR⁶ or S(O)ₖR⁴,
Het is a monocyclic or bicyclic, saturated, unsaturated or aromatic heterocyclic radical having 1-4 N, O and/or S atoms, bonded via N or C, which may be unsubstituted, monosubstituted or disubstituted or trisubstituted by Hal, A, COOR⁴, CN, 1H-tetrazol-5-yl, CONHSO₂R⁵, NH₂, NHA, NAA′, NO₂ and/or ═O,
Hal is fluorine, chlorine, bromine or iodine,
k is 0, 1 or 2,
m is 0, 1 or 2, and
n is 1 or 2,
and the (Z)- and (E)-isomers and the salts of all isomers;
o) the compounds of the formula I described in WO 9842702 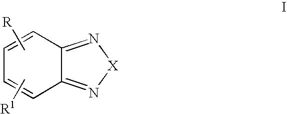
in which
R 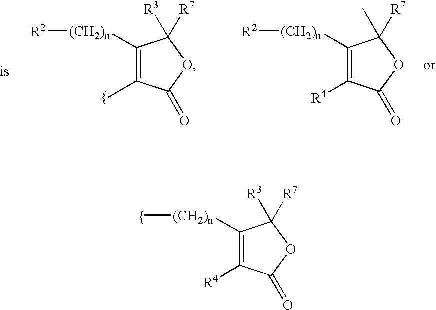
X and Y are each, independently of one another, O or S,
R¹ is H, Hal, OH, OA, A, alkylene-O-A, NO₂, NH., NH-acyl, SO₂NH₂, SO₂-A, SO₂NHA, CN or formyl,
R², R³ and R⁴ are each, independently of one another, a phenyl group which is unsubstituted or monosubstituted or polysubstituted by Hal, OH, OA, O-alkylene-R⁵, A, S-A, S-OA, SO₂A, S—OR⁵, SO₂R⁵, NO₂, NH₂, NHA, NA₂, NH-acyl, NHSO₂A NHSO₂R⁵, NASOA, NASO₂—R⁵, NH(CO)NH₂, NH(CO)NHA, formyl, NH(CO)NHR⁵, NHCOOA, NA-acyl, NHCOOCH₂R⁵, NHSO₂CH₂R⁵, NHCOO-alkylene-OA, NH(CO)NA₂, 1-piperidinyl-CO—NH, 1-pyrrolidinyl-CONH, O(CH₂),COOA, O(CH₂),COOH, O(CH₂)ₙOH, O(CH₂)ₙOA, CH₂OH, CH₂OA, COOH, COOA, CH₂COOH or CH₂COOA, 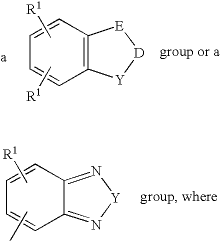
R² is additionally A or cycloalkyl,
R⁵ is a phenyl group which is unsubstituted or monosubstituted or polysubstituted by Hal, OH, OA, A, 8-A, NO₂, NH₂, NHA, NA₂, NH-acyl, NHSO₂A, NASO₂A, NH(CO)NH₂, NH(CO)NHA, formyl, NHCOOA, NA-acyl, NHCOO-alkylene-OA, NH(CO)NA₂, N-piperidinyl-CO—NH, N-pyrrolidinyl-CONH, O(CH₂)ₙCOOA, O(CH₂)ₙCOOH, O(CH₂)ₙOH, O(CH₂)ₙOA, CH₂OH, CH₂OA, COOH, COOA, CH₂COOH or CH₂COOA,
A is alkyl having 1-6 carbon atoms, in which one or two CH₂ groups may be replaced by O or S atoms or by —CR⁶CR⁶— groups and/or 1-7H atoms may be replaced by F,
D is carbonyl or IC(R⁶R^{6′}))ₘ,
E is CH₂, S or O,
R⁶ and R^{6′} are each, independently of one another, H, F or A,
R⁷ is —O—C(═Y)-NH—R¹,
R⁸ is alkyl having 1-10 carbon atoms which is unsubstituted or monosubstituted or disubstituted by R¹ and in which 1-2 carbon atoms may be replaced by 0 and/or S, and/or may be substituted by ═O, or cycloalkyl, in which 1-2 carbon atoms may be replaced by N, O and/or S,
R⁹ is phenyl which is unsubstituted or monosubstituted or disubstituted by Hal, or is naphthyl, A-O—C(═O)— or Hal,
Hal is fluorine, chlorine, bromine or iodine,
n is 0, 1 or 2, and
m is 1 or 2,
and salts thereof;
p) the compounds of the formula I described in WO 9842709 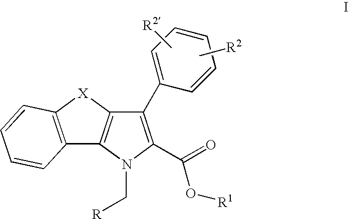
in which
X is N—R³, O or S,
R is 2,1,3-benzothiadiazol-4- or 5-yl or 2,1-benzoisothiazol-5- or 6-yl, each of which is unsubstituted or monosubstituted or disubstituted by R² and/or R²′, or phenyl which is unsubstituted or monosubstituted, disubstituted or trisubstituted by R² and/or R²′,
R¹ is H or A,
R² and R^{2′} are each, independently of one another, H, A, OH, OA,
Hal, OCF₃, OCHF₂, —O—CO-A, —O-alkylene-COOR¹, —O-alkylene-CH₂—OR¹, or OCH₂-phenyl or —O—CO-phenyl, each of which is unsubstituted or monosubstituted or disubstituted in the phenyl part by R⁴ and/or R⁴′,
R² and R^{2′} together are alternatively —OCH₂O—, —OCH₂CH₂O— or —OCH₂CH₂—,
R³ is H, A, alkylene-O-A, —CO-OA, or alkylene-phenyl which is unsubstituted or monosubstituted or disubstituted in the phenyl part by R⁴ and/or R^{4′},
R⁴ and R^{4′} are each, independently of one another, H, A, OH, OA,
Hal, COOR¹ or CH₂OR¹,
A is alkyl having 1-6 carbon atoms,
Hal is fluorine, chlorine, bromine or iodine,
and their salts;
q) the compounds of the formula I described in WO 9905132 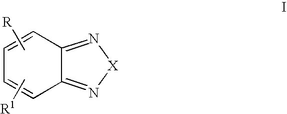
in which
R 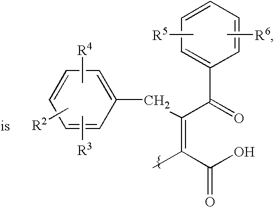
X is O or S,
R¹ is H, Hal, OA or A,
R², R³, R⁵, and R⁶ are each, independently of one another, H, Hal, A, OA or R⁴,
R⁴ is —O—(CH₂)ₙ-Cy,
Cy is cycloalkyl having 3-8 carbon atoms,
A is alkyl having 1-6 carbon atoms, in which one or two CH₂ groups may be replaced by O or S atoms or by —CR⁵═CR⁵— groups and/or 1-7H atoms may be replaced by F.
R⁵ and R^{5′} are each, independently of one another, H, F or A,
Hal is fluorine, chlorine, bromine or iodine,
n is 0, 1 or 2 ,
or a tautomeric cyclised form, and the (E)-isomers and the salts of all isomers.

42. Pharmaceutical formulation according to one of the preceding claims, comprising one or more excipients and/or assistants.

43. Use of a pharmaceutical preparation according to one of claims 31 to 42 for the preparation of a medicament for the treatment of angina, high blood pressure, high pulmonary pressure, congestive heart failure (CHF), chronic obstructive pulmonary disease (COPD), cor pulmonale, dextrocardiac insufficiency, atherosclerosis, conditions of reduced patency of the heart vessels, peripheral vascular diseases, strokes, bronchitis, allergic asthma, chronic asthma, allergic rhinitis, glaucoma, irritable bowel syndrome, tumours, renal insufficiency, liver cirrhosis, erectile dysfunction and for the treatment of female sexual disorders.

44. Use according to claim 43 for the preparation of a medicament for the treatment of high pulmonary pressure, congestive heart failure (CHF), chronic obstructive pulmonary disease (COPD), cor pulmonale and/or dextrocardiac insufficiency.

45. Set (kit) consisting of separate packs of
(a) an effective amount of 5-[4-(3-chloro-4-methoxybenzylamino)-5,6,7,8-tetrahydro-[1]-benzothieno[2,3-d]pyrimidin-2-yl]valeric acid and/or physiologically acceptable salts and/or solvates thereof and
(b) an effective amount of an endothelin receptor antagonist.
